(19) United States
(12) Patent Application Publication
(10) Pub. No.: US 2019/0167676 A1
(43) Pub. Date: Jun. 6, 2019

(54) METHODS FOR IMPROVING RESISTANCE TO SKELETAL MUSCLE FATIGUE

(71) Applicant: Cytokinetics, Inc., South San Francisco, CA (US)

(72) Inventors: Fady MALIK, South San Francisco, CA (US); Jeffrey R. JASPER, South San Francisco, CA (US); Adam KENNEDY, South San Francisco, CA (US); Darren T. HWEE, South San Francisco, CA (US)

(21) Appl. No.: 16/206,747

(22) Filed: Nov. 30, 2018

Related U.S. Application Data
(63) Continuation of application No. 14/391,087, filed on Oct. 7, 2014.
(60) Provisional application No. 61/735,809, filed on Dec. 11, 2012.
(60) Provisional application No. 61/693,061, filed on Aug. 24, 2012.
(60) Provisional application No. 61/646,842, filed on May 14, 2012.
(60) Provisional application No. 61/623,003, filed on Apr. 11, 2012.

(51) Int. Cl. A61K 31/4985; A61K 45/06; A61K 31/437; A61K 31/506

(52) U.S. Cl. CPC A61K 31/4985; A61K 31/506; A61K 31/437; A61K 45/06

(57) ABSTRACT

Provided are compounds, compositions and methods for improving resistance to skeletal muscle fatigue comprising administering an effective amount of a skeletal muscle troponin activator. Also provided are methods for improving resistance to fatigue, improving physical endurance, or reducing exercise intolerance in a subject suffering from a condition associated with muscle fatigue or weakness, such as heart failure.

This application claims priority to U.S. Application No. 61/623,003, filed Apr. 11, 2012, U.S. Application No. 61/646,842, filed May 14, 2012, U.S. Application No. 61/693,061, filed Aug. 24, 2012, and U.S. Application No. 61/735,809, filed Dec. 11, 2012, each of which is incorporated by reference in its entirety.

Muscle fatigue is often defined as a reversible decline of force production during activity. Muscle fatigue consists of a complex interplay between central and peripheral fatigue. The extent of peripheral muscle fatigue is dependent on several factors including muscle fiber type and stimulation frequency. Tetanic force often declines a small amount soon after muscle stimulation begins, then force declines slowly and finally there is a rapid decline to a fraction of initial force. In various human studies, fatigue appears to be only partially influenced by inadequate action potentials or inadequate voltage-sensor activation of the sarcoplasmic reticulum (SR), but rather due to metabolic changes within the muscle fibers that alter contractile function. Thus, during increased muscle function, muscle temperature rises, intracellular pH drops, inorganic phosphate (Pi) and ADP concentrations rise due to breakdown of ATP and creatine phosphate, and concentrations of reactive oxygen species (ROS) rise. Each of these factors decrease fast-twitch skeletal muscle stimulated tension, contraction velocity and power.

Intracellular Pi and protons (H⁺) directly inhibit muscle cross-bridge force and, importantly, shift the force-pCa relationship to the right. The decreased force-pCa relationship means that higher free intracellular Ca²⁺ concentrations are required to elicit a given tension. Elevated ROS also reduce Ca²⁺ sensitivity of fast skeletal muscle myofibrils and play a role in the phenomenon of fatigue. During initiation of muscle contraction, the concentration of Ca²⁺ remains high and peak force is not altered by the decreased calcium sensitivity. As muscle contraction continues, Ca²⁺ transients drop and, because of the diminished Ca²⁺ sensitivity induced by Pi and H⁺ (and possibly ROS), force declines. This is the phenomenon of peripheral fatigue. (Allen D. G. et al. Physiol Rev 88: 287-332, 2008; Fitts, J App Physiol 104:551-558, 2008; Allen, Appl Physiol. 2011 August;111(2):358-66).

In addition to peripheral fatigue involving changes at or distal to the neuromuscular junction described above, muscle fatigue can also be influenced by central fatigue (S. C. Gandevia, Physiol. Rev., 81: 1726-1788, 2001). Central fatigue can be described as a progressive reduction in voluntary activation of muscle during exercise and involves a conscious sensation of fatigue and perception of effort. This perception of effort has been associated with various somatosensory signals, emotional state, discomfort, pain, thermal stress and thirst (Noakes et al. Br J Sports Med. August; 38(4): 511-514, 2004, J. W. Williamson, Exp Physiol 95: 1043-1048, 2010). This integrated mechanism works to preserve the integrity of the system by initiating muscle fatigue through inhibition of muscle recruitment, and as a result, maximal voluntary strength can be below the true maximal muscle force. Alteration in perceived effort, as is possible in the presence of a skeletal troponin activator, can mitigate fatigue.

The means to decrease fatigue in certain situations has therapeutic potential, especially in a number of disease settings, including heart failure. Muscle function can become compromised in disease by many mechanisms. Accordingly, there is a need for the development of new compounds that modulate skeletal muscle contractility and of new methods for improving resistance to skeletal muscle fatigue.

### SUMMARY

Provided are compounds, compositions and methods for improving resistance to skeletal muscle fatigue. In some embodiments, the method comprises administering to a subject an effective amount of a skeletal muscle troponin activator. In some embodiments, the skeletal muscle fatigue is selected from central fatigue, peripheral fatigue, and a combination thereof. In some embodiments, the skeletal muscle troponin activator is a fast skeletal muscle troponin activator. In some embodiments, the subject is suffering from a condition selected from peripheral artery disease, claudication, and muscle ischemia.

Also provided are methods for improving physical endurance or reducing exercise intolerance in a subject, comprising administering to the subject an effective amount of a skeletal muscle troponin activator.

Also provided are methods of improving resistance to fatigue in a skeletal muscle, comprising contacting the skeletal muscle with a skeletal muscle troponin activator, wherein the skeletal muscle troponin activator increases submaximal tension in the skeletal muscle.

Also provided are methods of improving resistance to fatigue in a skeletal muscle, comprising contacting the skeletal muscle with a skeletal muscle troponin activator, wherein the skeletal muscle troponin activator reduces the intracellular calcium required by the skeletal muscle to generate force.

Also provided are methods for improving resistance to fatigue in a patient suffering from heart failure, comprising administering to the patient an effective amount of a skeletal muscle troponin activator. In some embodiments, the method comprises administering to a subject an effective amount of a skeletal muscle troponin activator. In some embodiments, the skeletal muscle troponin activator is a fast skeletal muscle troponin activator. Similarly, the compounds, compositions and methods described and/or disclosed herein may be used to improve exercise tolerance.

Also provided are methods for treating exercise intolerance associated with heart failure in a subject, comprising administering to the subject an effective amount of a skeletal muscle troponin activator.

Also provided are methods for improving physical endurance of a patient suffering from heart failure, comprising administering to the subject an effective amount of a skeletal muscle troponin activator.

Also provided are methods for increasing the function, activity, efficiency, sensitivity to calcium, or time to fatigue of skeletal muscle of a patient suffering from heart failure, comprising administering to the patient an effective amount of a skeletal muscle troponin activator.

Also provided are methods for improving skeletal muscle function of a patient suffering from heart failure, comprising administering to the patient an effective amount of a skeletal muscle troponin activator.

Also provided are methods for improving resistance to skeletal muscle fatigue in a subject in need thereof, comprising administering to the subject an effective amount of a skeletal muscle troponin activator, wherein the improvement in resistance to fatigue in the subject is determined by a bilateral heel-raise test. In some embodiments, the bilateral heel-raise test comprises: instructing the subject to perform heel raises at regular intervals; and measuring one or more parameters selected from time to claudication onset, number of heel raises to claudication onset, work to claudication onset, time to maximal claudication fatigue, number of heel raises to maximal claudication fatigue, and work to maximal claudication fatigue, wherein an increase in one or more of the parameters indicates an improvement in resistance to fatigue in the subject.

Also provided are methods for determining the efficacy of a skeletal muscle troponin activator in improving resistance to skeletal muscle fatigue in a subject, comprising administering to the subject a bilateral heel-raise test.

In some embodiments of any of the methods described herein, the skeletal muscle troponin activator is a fast skeletal muscle troponin activator. In some embodiments, the fast skeletal muscle activator selectively activates fast skeletal muscle.

In some embodiments, the skeletal muscle troponin activator is a compound of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, as defined herein.

In some embodiments, the skeletal muscle troponin activator is a compound of Formula A or B, as defined herein.

Other aspects and embodiments will be apparent to those skilled in the art from the following detailed description.

### BRIEF DESCRIPTION OF THE FIGURES

FIG. 1 is a graph showing the effect of the skeletal muscle troponin activator Compound A on sub-maximal force development in a rat flexor digitorum brevis muscle in vitro.

FIG. 2 is a graph showing the effect of Compound A on fatigue in a rat flexor digitorum brevis muscle in vitro.

FIG. 3 is a graph showing the effect of Compound A on relaxation time in a rat flexor digitorum brevis muscle in vitro. The upper plot is for relaxation time and the lower plot is for force.

FIG. 4 is a graph showing the effect of Compound A on fatigue in a rat extensor digitorum longus muscle in situ. The upper line is for Compound A and the lower line is for vehicle.

FIG. 5 is a graph showing the effect of Compound A on time to fatigue in a rat flexor digitorum brevis muscle after femoral artery ligation in vitro. The lower plot is for FAL; the middle plot is for FAL+≤0.5 mg/kg Compound A; the upper plot is for FAL+1 mg/kg Compound A.

FIG. 6A is a graph showing the effect of Compound A on the isometric force-frequency relationship in a rat plantorflexor muscle in situ.

FIG. 6B is a graph showing the effect of Compound A on the isokinetic force-frequency relationship in a rat plantorflexor muscle in situ.

FIG. 6C is a graph showing the effect of Compound A on the force-velocity relationship in a rat plantorflexor muscle in situ.

FIG. 6D is a graph showing the effect of Compound A on the power output in a rat plantorflexor muscle in situ.

FIG. 6E is a graph showing the effect of Compound A on force generation during an isokinetic fatigue protocol in a rat plantorflexor muscle in situ. The upper plot is for vehicle; the lower plot is for Compound A (3 mg/kg)

FIG. 7A is a graph showing the effect of the skeletal muscle troponin activator Compound B on the isometric force-frequency relationship in a rat plantorflexor muscle in situ. At each frequency, the bar on the left is for vehicle and the bar on the right is for Compound B.

FIG. 7B is a graph showing the effect of Compound B on the isokinetic force-frequency relationship in a rat plantorflexor muscle in situ. At each frequency, the bar on the left is for vehicle and the bar on the right is for Compound B.

FIG. 7C is a graph showing the effect of Compound B on the force-velocity relationship in a rat plantorflexor muscle in situ.

FIG. 7D is a graph showing the effect of Compound B on the power output in a rat plantorflexor muscle in situ.

FIG. 7E is a graph showing the effect of Compound B on force generation during an isokinetic fatigue protocol in a rat plantorflexor muscle in situ. The upper plot is for Compound B; the lower plot is for vehicle.

FIG. 7F is a graph showing the effect of Compound B on force generation during an isokinetic fatigue protocol at a stimulation frequency calculated to provide 50% of maximum kinetic tension in a rat plantorflexor muscle in situ.

FIG. 8 is a graph showing the effect of Compound A on grid hang time endurance in healthy rats.

FIG. 9 is a graph showing the effect of Compound A on rotarod running endurance in healthy rats.

FIG. 10A is a graph showing the effect of Compound A on treadmill running time in healthy rats.

FIG. 10B is a graph showing the effect of Compound A on treadmill running distance in healthy rats.

FIG. 11 depicts the lateral aspect of the ankle on the dominant leg instrumented with an electro-mechanical goniometer to assess ankle angle position and range of motion in the bilateral heel test.

FIG. 12 is a graph showing the mean plasma concentrations of Compound A in human subjects over time. Mean plasma Compound A concentrations showed relatively dose proportional increases. The top plot is for 750 mg; the middle plot is for 500 mg; the lower plot is for 375 mg. The plot for 0 mg is falls between the middle and lower plots.

FIG. 13A shows graphs depicting the clinical time-to-endpoint results of a heel raise test in human subjects.

FIG. 13B shows graphs depicting the clinical repititions-to-endpoint results of a heel raise test in human subjects.

FIG. 13C shows graphs depicting the clinical time-to-endpoint results of a heel raise test in human subjects.

FIG. 14 shows graphs depicting the relationship of pharmacodynamic measures to plasma Compound A concentrations in a heel raise test in human subjects. The PK/PD analysis shows a strong relationship between Compound A plasma concentrations and outcomes.

FIG. 15A is a graph showing the results of a 6-minute walk test with placebo-corrected change from baseline by Compound A dose.

FIG. 15B is a graph showing the results of the 6-minute walk test with placebo-corrected change from baseline by Compound A plasma concentration.

FIG. 16 shows the effect of Compound C on running time in a fatigue-rotarod test in healthy rats.

FIG. 17 shows the percent fractional shortening determined by echocardiography in a rat model of heart failure (ligation of left anterior descending (LAD) coronary artery).

FIG. 18 shows the effect of Compound C in on running time in a fatigue-rotarod test in an LAD rat model of heart failure.

FIG. 19 shows the effect that Compound C has on the force-frequency relationship for skinned soleus muscle fibers in sham rats (top panel) and LAD rats (bottom panel).

FIG. 20 shows the effect that treatment with Compound C or vehicle has on the force-frequency relationship for skinned soleus muscle fibers in sham rats (left panels) and LAD rats (right panels) versus baseline.

FIG. 21 shows the change in force-frequency response between baseline and subsequent treatment with Compound C in sham (top panel) and LAD (bottom panel) rats.

FIG. 22 shows the effect of Compound C on the relationship between force and Ca²⁺ concentration for skinned extensor digitorum longus (EDL) muscle fiber from sham and LAD rats. The plots on the left are for sham+3 μM Compound C and for LAD+3 μM Compound C. The plots on the right are for sham and for LAD.

FIG. 23 shows the effect of Compound C on the relationship between force and Ca²⁺ concentration for skinned diaphragm muscle fiber from sham and LAD rats. The plots on the left are for sham+3 μM Compound C and for LAD+3 μM Compound C. The plots on the right are for sham and for LAD.

FIG. 24 shows the baseline force-frequency relationship on diaphragm muscle fibers from sham and LAD rats.

FIG. 25 shows the effect of Compound C on the force-frequency relationship on diaphragm muscle fibers from sham (top panel) and LAD (bottom panel) rats.

### DETAILED DESCRIPTION

Throughout this application, unless the context indicates otherwise, references to a compound of a formula includes all subgroups of the formula defined herein, including all substructures, subgenera, preferences, embodiments, examples and particular compounds described herein.

References to a compound of a formula and subgroups thereof include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. “Crystalline form,” “polymorph,” and “novel form” may be used interchangeably herein, and are meant to include all crystalline and amorphous forms of the compound, including, for example, polymorphs, pseudopolymorphs, solvates (including hydrates), co-crystals, unsolvated polymorphs (including anhydrates), conformational polymorphs, and amorphous forms, as well as mixtures thereof, unless a particular crystalline or amorphous form is referred to. In some embodiments, references to a compound of a formula and subgroups thereof include polymorphs, solvates, co-crystals, isomers, tautomers and/or oxides thereof. In some embodiments, references to a compound include polymorphs, solvates, and/or co-crystals thereof. In some embodiments, references to a compound of a formula and subgroups thereof include isomers, tautomers and/or oxides thereof. In some embodiments, references to a compound of a formula and subgroups thereof include solvates thereof. Similarly, the term “salts” includes solvates of salts of compounds.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

When a moiety is defined as being optionally substituted, it may be substituted as itself or as part of another moiety. For example, if R^{x} is defined as “C₁₋₆ alkyl or OC₁₋₆ alkyl, wherein C₁₋₆ alkyl is optionally substituted with halogen”, then both the C₁₋₆ alkyl group alone and the C₁₋₆ alkyl that makes up part of the OC₁₋₆ alkyl group may be substituted with halogen.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes npropyl and isopropyl. “Lower alkyl” refers to alkyl groups having one to seven carbons. In certain embodiments, “lower alkyl” refers to alkyl groups having one to six carbons. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is a subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group.

“Haloalkyl” includes straight and branched carbon chains having the indicated number of carbon atoms (e.g., 1 to 6 carbon atoms) substituted with at least one halogen atom. In instances wherein the haloalkyl group contains more than one halogen atom, the halogens may be the same (e.g., dichloromethyl) or different (e.g., chlorofluoromethyl). Examples of haloalkyl groups include, but are not limited to, chloromethyl, dichloromethyl, trichloromethyl, fluoromethyl, difluoromethyl, trifluoromethyl, chlorofluoromethyl, 2-fluoroethyl, 2,2-difluoroethyl, 2,2,2-trifluoroethyl, 1,2-difluoroethyl, 2-chloroethyl, 2,2-dichloroethyl, 2,2,2-trichloroethyl, 1,2-dichloroethyl, pentachloroethyl, and pentafluoroethyl.

“Alkenyl” refers to an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2en-1-yl (allyl), prop-2-en-2-yl; butenyls such as but-1-en-1-yl, but-1-en-2-yl, 2-methylprop-1-en-1-yl, but-2-en-1-yl, but-2-en-1-yl, but-2-en-2-yl, buta-1,3-dien-1-yl, buta-1,3dien-2-yl; and the like. In certain embodiments, an alkenyl group has from 2 to 20 carbon atoms and in other embodiments, from 2 to 6 carbon atoms. “Lower alkenyl” refers to alkenyl groups having two to six carbons.

“Alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms. “Lower alkynyl” refers to alkynyl groups having two to six carbons.

“Cycloalkyl” indicates a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond derived by the removal of one molecule of hydrogen from adjacent carbon atoms of the corresponding cycloalkyl. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

The term “alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons.

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO²R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO²R^{a} and —SO₂NR^{b}R^{c}),

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, Cₗ-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄alkyl)C(O)(C₁-C₄alkyl), —N(C₁-C₄ alkyl)C(O)(phenyl), —C(0)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH2)_{y}OH, where y is an integer of 1-10, such as 1-4.

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}) optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OCONR^{b}R^{c}, —OP(O)(OR⁵)OR^{c}, sulfanyl (such as SR^{b}), sulfinyl (such as —SOR^{a}), and sulfonyl (such as —SO₂R^{a} and —SO₂NR^{b}R^{c}),

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses:

6-membered carbocyclic aromatic rings, for example, benzene;

bicyclic ring systems wherein at least one ring is carbocyclic and aromatic, for example, naphthalene, indane, and tetralin; and

tricyclic ring systems wherein at least one ring is carbocyclic and aromatic, for example, fluorene.

For example, aryl includes 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

“Aralkoxy” refers to the group —O-aralkyl. Similarly, “heteroaralkoxy” refers to the group —O-heteroaralkyl; “aryloxy” refers to —O-aryl; and “heteroaryloxy” refers to the group —O-heteroaryl.

“Aralkyl” refers to a residue in which an aryl moiety is attached to the parent structure via an alkyl residue. Examples include benzyl, phenethyl, phenylvinyl, phenylallyl and the like. “Heteroaralkyl” refers to a residue in which a heteroaryl moiety is attached to the parent structure via an alkyl residue. Examples include furanylmethyl, pyridinylmethyl, pyrimidinylethyl and the like.

“Halogen” or “halo” refers to fluorine, chlorine, bromine or iodine. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

“Heteroaryl” encompasses:

5- to 7-membered aromatic, monocyclic rings containing one or more, for example, from 1 to 4, or in certain embodiments, from 1 to 3, heteroatoms chosen from N, O, and S, with the remaining ring atoms being carbon;

bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and

tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 5- to 7-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein

Substituted heteroaryl also includes ring systems substituted with one or more oxide (—O⁻) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring may be saturated or have one or more carbon-carbon double bonds. Suitable heterocycloalkyl groups include, for example (as numbered from the linkage position assigned priority 1), 2-pyrrolidinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (═O) or oxide (−O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteratoms independently selected from oxygen, sulfur, and nitrogen and is not aromatic.

“Heterocycloalkenyl” indicates a non-aromatic ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). When nitrogen is present in a heterocycloalkenyl ring, it may, where the nature of the adjacent atoms and groups permits, exist in an oxidized state (i.e., N⁺—O⁻). Additionally, when sulfur is present in a heterocycloalkenyl ring, it may, where the nature of the adjacent atoms and groups permits, exist in an oxidized state (i.e., S⁺—O⁻ or SO₂—). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group. Examples of polycyclic heterocycloalkenyl groups consisting of a heterocycloalkenyl group fused to an aromatic ring are described below.

Examples of polycyclic rings consisting of an aromatic ring (e.g., aryl or heteroaryl) fused to a non-aromatic ring (e.g., cycloalkyl, cycloalkenyl, heterocycloalkyl, heterocycloalkenyl) include indenyl, 2,3-dihydro-1H-indenyl, 1,2,3,4-tetrahydronaphthalenyl, benzo[1,3]dioxolyl, tetrahydroquinolinyl, 2,3-dihydrobenzo[1,4]dioxinyl, indolinyl, isoindolinyl, 2,3-dihydro-1H-indazolyl, 2,3-dihydro-1H-benzo[d]imidazolyl, 2,3-dihydrobenzofuranyl, 1,3-dihydroisobenzofuranyl, 1,3-dihydrobenzo[c]isoxazolyl, 2,3-dihydrobenzo[d]isoxazolyl, 2,3-dihydrobenzo[d]oxazolyl, 2,3-dihydrobenzo[b]thiophenyl, 1,3-dihydrobenzo[c]thiophenyl, 1,3-dihydrobenzo[c]isothiazolyl, 2,3-dihydrobenzo[d]isothiazolyl, 2,3-dihydrobenzo[d]thiazolyl, 5,6-dihydro-4H-cyclopenta[d]thiazolyl, 4,5,6,7-tetrahydrobenzo[d]thiazolyl, 5,6-dihydro-4H-pyrrolo[3,4-d]thiazolyl, 4,5,6,7-tetrahydrothiazolo[5,4-c]pyridinyl, indolin-2-one, indolin-3-one, isoindolin-1-one, 1,2-dihydroindazol-3-one, 1H-benzo[d]imidazol-2(3H)-one, benzofuran-2(3H)-one, benzofuran-3(2H)-one, isobenzofuran-1(3H)-one, benzo[c]isoxazol-3(1H)-one, benzo[d]isoxazol-3(2H)-one, benzo[d]oxazol-2(3H)-one, benzo[b]thiophen-2(3H)-one, benzo[b]thiophen-3(2H)-one, benzo[c]thiophen-1(3H)-one, benzo[c]isothiazol-3(1H)-one, benzo[d]isothiazol-3(2H)-one, benzo[d]thiazol-2(3H)-one, 4,5-dihydropyrrolo[3,4-d]thiazol-6-one, 1,2-dihydropyrazolo[3,4-d]thiazol-3-one, quinolin-4(3H)-one, quinazolin-4(3H)-one, quinazoline-2,4(1H,3H)-dione, quinoxalin-2(1H)-one, quinoxaline-2,3(1H,4H)-dione, cinnolin-4(3H)-one, pyridin-2(1H)-one, pyrimidin-2(1H)-one, pyrimidin-4(3H)-one, pyridazin-3(2H)-one, 1H-pyrrolo[3,2-b]pyridin-2(3H)-one, 1H-pyrrolo[3,2-c]pyridin-2(3H)-one, 1H-pyrrolo[2,3-c]pyridin-2(3H)-one, 1H-pyrrolo[2,3-b]pyridin-2(3H)-one, 1,2-dihydropyrazolo[3,4-d]thiazol-3-one and 4,5-dihydropyrrolo[3,4-d]thiazol-6-one. As discussed herein, whether each ring is considered an aryl, heteroaryl, cycloalkyl, cycloalkenyl, heterocycloalkyl or heterocycloalkenyl group is determined by the atom through which the moiety is bound to the parent structure.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(.±.)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)— or (S)—. The present invention is meant to include all such possible isomers, including racemic mixtures, optically pure forms and intermediate mixtures. Optically active (R)— and (S)— isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

The stereochemistry depicted in the structures of cyclic meso compounds is not absolute; rather the stereochemistry is intended to indicate the positioning of the substituents relative to one another, e.g., cis or trans. For example,

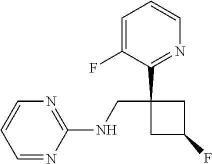 [SMILES: Fc1cccnc1[C@]1(CNc2ncccn2)C[C@H](F)C1]

is intended to designate a compound wherein the fluorine and pyridyl substituents on the cyclobutyl ring are in a cis configuration to one another, while

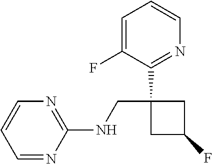 [SMILES: Fc1cccnc1[C@]1(CNc2ncccn2)C[C@@H](F)C1]

is intended to designate a compound wherein the fluorine and pyridyl substituents on the cyclobutyl ring are in a trans configuration to one another.

When a compound can exist as one or more meso isomers, all possible meso isomers are intended to be included. For example, the compound {[3-fluoro-1-(3-fluoro(2-pyridyl))cyclobutyl]methyl}pyrimidin-2-ylamine is intended to include both cis and trans meso isomers:

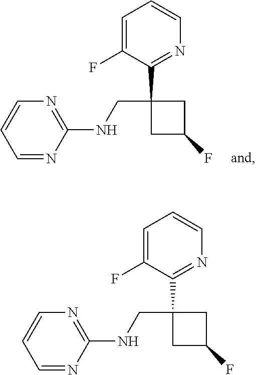 [SMILES: Fc1cccnc1[C@]1(CNc2ncccn2)C[C@H](F)C1 ; Fc1cccnc1[C@]1(CNc2ncccn2)C[C@@H](F)C1]

and mixtures thereof. Unless otherwise indicated, compounds described herein include all possible meso isomers and mixtures thereof.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconverision of pentane-2,4-dione and 4-hydroxypent-3-en-2-one tautomers. Another example of tautomerization is phenol-keto tautomerization. A specific example of phenol-keto tautomerization is the interconverision of pyridin-4-ol and pyridin-4(1H)-one tautomers. Compounds of certain of the disclosed formulas are tautomeric.

A leaving group or atom is any group or atom that will, under the reaction conditions, cleave from the starting material, thus promoting reaction at a specified site. Suitable examples of such groups unless otherwise specified are halogen atoms, mesyloxy, p-nitrobenzensulphonyloxy and tosyloxy groups.

Protecting group has the meaning conventionally associated with it in organic synthesis, i.e. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a hydroxy protected form is where at least one of the hydroxy groups present in a compound is protected with a hydroxy protecting group. Likewise, amines and other reactive groups may similarly be protected.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts that retain the biological effectiveness and properties of the compounds described herein and, which are not biologically or otherwise undesirable. In many cases, the compounds described herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “solvate” refers to a compound in physical association with one or more molecules of a pharmaceutically acceptable solvent. It will be understood that “a compound” encompass the compound, and solvates of that compound, as well as mixtures thereof.

A “chelate” is formed by the coordination of a compound to a metal ion at two (or more) points. The term “compound” is intended to include chelates of compounds. Similarly, “salts” includes chelates of salts and “solvates” includes chelates of solvates.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”.

The term “prodrug” refers to a substance administered in an inactive or less active form that is then transformed (e.g., by metabolic processing of the prodrug in the body) into an active compound. The rationale behind administering a prodrug is to optimize absorption, distribution, metabolism, and/or excretion of the drug. Prodrugs may be obtained by making a derivative of an active compound that will undergo a transformation under the conditions of use (e.g., within the body) to form the active compound. The transformation of the prodrug to the active compound may proceed spontaneously (e.g., by way of a hydrolysis reaction) or it can be catalyzed or induced by another agent (e.g., an enzyme, light, acid or base, and/or temperature). The agent may be endogenous to the conditions of use (e.g., an enzyme present in the cells to which the prodrug is administered, or the acidic conditions of the stomach) or the agent may be supplied exogenously. Prodrugs can be obtained by converting one or more functional groups in the active compound into another functional group, which is then converted back to the original functional group when administered to the body. For example, a hydroxyl functional group can be converted to a sulfonate, phosphate, ester or carbonate group, which in turn can be hydrolyzed in vivo back to the hydroxyl group. Similarly, an amino functional group can be converted, for example, into an amide, carbamate, imine, urea, phosphenyl, phosphoryl or sulfenyl functional group, which can be hydrolyzed in vivo back to the amino group. A carboxyl functional group can be converted, for example, into an ester (including silyl esters and thioesters), amide or hydrazide functional group, which can be hydrolyzed in vivo back to the carboxyl group. Examples of prodrugs include, but are not limited to, phosphate, acetate, formate and benzoate derivatives of functional groups (such as alcohol or amine groups) present in the compounds described herein.

The compounds described herein can be enriched isotopic forms, e.g., enriched in the content of ²H, 3H, ¹¹C, ¹³C and/or ¹⁴C. In some embodiments, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6(10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64(1-2), 9-32.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂B^{b}), aminocarbonyl (such as —CONR^{b}R^{c}), —OCOR^{b}, —OCO₂R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),

where

R^{a} is chosen from optionally substituted C₁-C₆ alkyl, optionally substituted cycloalkyl, optionally substituted heterocycloalkyl, optionally substituted alkenyl, optionally substituted alkynyl, optionally substituted aryl, and optionally substituted heteroaryl;

R^{b} is chosen from hydrogen, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “sulfanyl” refers to the groups: —S-(optionally substituted alkyl), —S-(optionally substituted cycloalkyl), —S-(optionally substituted aryl), —S-(optionally substituted heteroaryl), and —S-(optionally substituted heterocycloalkyl).

The term “sulfinyl” refers to the groups: —S(O)—H, —S(O)-(optionally substituted alkyl), —S(O)-(optionally substituted cycloalkyl), —S(O)-(optionally substituted amino), —S(O)-(optionally substituted aryl), —S(O)-(optionally substituted heteroaryl), and —S(O)-(optionally substituted heterocycloalkyl).

The term “sulfonyl” refers to the groups: —S(O₂)—H, —S(O₂)-(optionally substituted alkyl), —S(O₂)-(optionally substituted cycloalkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

The term “active agent” is used to indicate a compound that has biological activity. In some embodiments, an “active agent” is a compound having therapeutic utility. In some embodiments, the compound enhances at least one aspect of skeletal muscle function or activity, such as power output, skeletal muscle force, skeletal muscle endurance, oxygen consumption, efficiency, and/or calcium sensitivity.

Compounds also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including an hydrates), conformational polymorphs, and amorphous forms, as well as mixtures thereof, unless a particular crystalline or amorphous form is referred to.

Chemical entities include, but are not limited to, compounds of the disclosed formulas, and all pharmaceutically acceptable forms thereof. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

The terms “patient” and “subject” refer to an animal, such as a mammal bird or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

As used herein, “skeletal muscle” includes skeletal muscle tissue as well as components thereof, such as skeletal muscle fibers, the myofibrils comprising the skeletal muscle fibers, the skeletal sarcomere which comprises the myofibrils, and the various components of the skeletal sarcomere described herein, including skeletal myosin, actin, tropomyosin, troponin C, troponin I, troponin T and fragments and isoforms thereof. In some embodiments, “skeletal muscle” includes fast skeletal muscle tissue as well as components thereof, such as fast skeletal muscle fibers, the myofibrils comprising the fast skeletal muscle fibers, the fast skeletal sarcomere which comprises the myofibrils, and the various components of the fast skeletal sarcomere described herein, including fast skeletal myosin, actin, tropomyosin, troponin C, troponin I, troponin T and fragments and isoforms thereof. Skeletal muscle does not include cardiac muscle or a combination of sarcomeric components that occurs in such combination in its entirety in cardiac muscle.

As used herein, the term “therapeutic” refers to the ability to modulate the contractility of fast skeletal muscle. As used herein, “modulation” (and related terms, such as “modulate”, “modulated”, “modulating”) refers to a change in function or efficiency of one or more components of the fast skeletal muscle sarcomere, including myosin, actin, tropomyosin, troponin C, troponin I, and troponin T from fast skeletal muscle, including fragments and isoforms thereof, as a direct or indirect response to the presence of a compound described herein, relative to the activity of the fast skeletal sarcomere in the absence of the compound. The change may be an increase in activity (potentiation) or a decrease in activity (inhibition), and may be due to the direct interaction of the compound with the sarcomere, or due to the interaction of the compound with one or more other factors that in turn affect the sarcomere or one or more of its components. In some embodiments, modulation is a potentiation of function or efficiency of one or more components of the fast skeletal muscle sarcomere, including myosin, actin, tropomyosin, troponin C, troponin I, and troponin T from fast skeletal muscle, including fragments and isoforms thereof. Modulation may be mediated by any mechanism and at any physiological level, for example, through sensitization of the fast skeletal sarcomere to contraction at lower Ca²⁺ concentrations. As used herein, “efficiency” or “muscle efficiency” means the ratio of mechanical work output to the total metabolic cost.

The term “muscle fatigue” or “skeletal muscle fatigue” refers to a reduction in contractile capacity following repeat-use and represents a combination of central fatigue (limitations of the central and peripheral nervous system to sustain activity) and peripheral fatigue (intrinsic loss of muscle function such as a reduced effectiveness of excitation-contraction coupling). Together, these result in reduced muscle performance under fatiguing conditions. Diminished resistance to fatigue is a common symptom of multiple diseases with a broad array of causes. In this context, fatigue constitutes a major factor in quality of life in conditions such as ALS, COPD, multiple sclerosis, myocardial infarction, claudication, myasthenia gravis, anemia, and chronic fatigue syndrome.

The term “value” refers to a numerical result.

The term “parameter” refers to a measurable factor. The measurements obtained from accessing a parameter are the parameter values. Parameters can include, for example, time to claudication onset, number of heel raises to claudication onset, work to claudication onset, time to maximal claudication fatigue, number of heel raises to maximal claudication fatigue, and work to maximal claudication fatigue

The term “work to claudication onset” or “work to maximal claudication fatigue” refers to the work performed before the onset of claudication or maximal claudication fatigue. The work can be defined as the value from the formula: sine * foot length * body mass where θ is equal to the degree of plantar flexion. The degree of plantar flexion can be measured with the aid of instruments such as a goniometer, for example.

The term “therapeutically effective amount” or “effective amount” refers to that amount of a compound selected from the disclosed formulas that is sufficient to effect treatment, as defined below, when administered to a mammal in need of such treatment. The therapeutically effective amount will vary depending upon the subject and disease condition being treated, the weight and age of the subject, the severity of the disease condition, the particular compound selected from the disclosed formulas, the dosing regimen to be followed, timing of administration, the manner of administration and the like, all of which can readily be determined by one of ordinary skill in the art.

“Treatment” or “treating” means any treatment of a disease in a patient, including:
(a) preventing the disease, that is, causing the clinical symptoms of the disease not to develop;
(b) inhibiting the disease;
(c) slowing or arresting the development of clinical symptoms; and/or
(d) relieving the disease, that is, causing the regression of clinical symptoms.

As used herein, “power output” of a muscle means work/cycle time and may be scaled up from PoLo/cycle time units based on the properties of the muscle. Power output may be modulated by changing, for example, activating parameters during cyclical length changes, including timing of activation (phase of activation) and the period of activation (duty cycle.)

“ATPase” refers to an enzyme that hydrolyzes ATP. ATPases include proteins comprising molecular motors such as the myosins.

As used herein, “selective binding” or “selectively binding” refers to preferential binding to a target protein in one type of muscle or muscle fiber as opposed to other types. For example, a compound selectively binds to fast skeletal troponin C if the compound preferentially binds troponin C in the troponin complex of a fast skeletal muscle fiber or sarcomere in comparison with troponin C in the troponin complex of a slow muscle fiber or sarcomere or with troponin C in the troponin complex of a cardiac sarcomere.

The compounds described herein selectively sensitize fast skeletal muscle to calcium by binding to the troponin complex. By increasing the calcium sensitivity of the troponin-tropomyosin regulatory complex, which is the calcium sensor within the sarcomere that regulates the actin-myosin force-generating interaction, the compounds improve muscle force generation. As a consequence of this activity on the troponin-tropomyosin complex, the compounds amplify the response of muscle to neuromuscular input and also decrease the fatigability of muscle. Thus, the compounds will improve muscle strength in the face of fatigue in healthy subjects as well as subjects suffering from neuromuscular disorders or other conditions marked by muscle weakness.

Skeletal muscle fatigue is a complex phenomenon that, in general terms, can involve the central nervous system, motor neuron firing, muscle cell depolarization/action potential propagation, release of sarcoplasmic reticulum (SR) calcium, activation of troponin on the thin filaments and cross-bridge cycling of myosin interacting with actin to generate force. Typically, in humans fatigue is thought to minimally involve the central nervous system or the neuromuscular junction, i.e. “central fatigue” but rather primarily involves myocytes themselves. Fatigue is often categorized as either low frequency fatigue due to repeated tetanic stimulation or high frequency fatigue due to continuous high frequency stimulation. Tension declines slowly during a sustained maximum voluntary contraction (low frequency fatigue).

In prolonged skeletal muscle contraction, Pi and ADP concentrations rise due to breakdown of ATP and creatine phosphate. In numerous studies, Pi has been demonstrated to be an important factor to decrease muscle function (N. C. Millar and E. Homsher. J. Biol. Chem. Vol. 265, No. 33, Issue of November 25, pp. 20234-20240,199O; Allen D. G. et al. Physiol Rev; 88:287-332. 2008). Because Pi is released from actin-myosin crossbridges at a position in the cross-bridge cycle associated with force production, elevated Pi levels are presumed to accelerate the backward rate of this step and thereby reduce muscle force (Takagi Y. et al. Philos Trans R Soc Lond B Biol Sci. 2004 Dec. 29; 359(1452):1913-1920; Fitts et al. J Appl Physiol 104:551-558, 2008). In addition to decreased force, Pi decreases Ca²⁺ sensitivity of troponin (H. Westerblad et al. Cellular mechanisms of fatigue in skeletal muscle. Am. J. Physiol. 261 (Cell Physiol. 30): C195-C209, 1991). For example, in skinned rabbit psoas muscle, as Pi in the bath was increased from 0.2 mM to 13.8 mM, Ca²⁺ sensitivity toward tension development was reduced from pCa=6.81 to pCa=6.42 and the Hill slope increased from 2.5 to 4.74 (Millar 1990). Compared to low temperature (10-20° C.), when skinned muscle fiber work was conducted at near physiological temperatures (˜30° C.), the negative effects on Ca²⁺ sensitivity in fast skeletal muscle fibers were amplified. Thus, at lower Ca²⁺ concentrations (pCa>5.8), high levels of Pi appear to reduce the force per cross-bridge and significantly increase the free Ca²⁺ level required for half-maximal peak force (lower pCa₅₀) (E. P. Debold et al. Am J Physiol Cell Physiol 290:C1041-C1050, 2006.). Furthermore Allen and colleagues explored the Pi reduction in Ca²⁺ sensitivity of contraction in intact mouse muscles (Allen, D G. et al. J. Appl Physiol 111: 358-366, 2011). In those studies, not only did Pi increase as muscle force decreased (as measured by ³¹P-NMR) but tetanic myocyte and sarcoplasmic reticulum (SR) calcium levels also dropped in relation to force during fatiguing stimulation protocols. Thus, during fatigue Pi also decreases Ca²⁺ release from the SR (Allen 2008) and decreases SR calcium levels, likely by precipitating SR Ca²⁺ (D. G. Allen and H. Westerblad Journal of Physiology (2001), 536.3, pp. 657-665). Taken together, the reduced Ca²⁺ release from the SR, the Pi-induced rightward-shift of the pCa-force relationship and the direct negative effects of Pi on cross-bridge cycling likely combine to synergistically reduce muscle force and power output. Debold and colleagues (2006) gave the example that if intracellular Ca²⁺ were to be reduced from pCa 5.0 to 6.0 (which would be a typical drop in free intracellular Ca²⁺ that might be observed after prolonged stimulation of intact muscle fibers) at physiological temperature, and in the presence of 30 mM Pi (typical in fatigued fibers), force would be reduced by about 90% in type II fibers. It is this drop in muscle performance as a function of decreased free intracellular Ca²⁺ and diminished calcium sensitivity of fast muscle fibers that suggests a potential role for fast skeletal troponin activators to mitigate fatigue.

Numerous studies have demonstrated the importance of impaired SR Ca²⁺ release into the myoplasm for the development of fatigue (D. G. Allen et al. Journal of Physiology (1989), 415. pp. 433-458; Allen et al. 1995 Exp. Physio.I 80, 497-527; Favero 1999 J Appl Physiol. 87: 471-483). Ca²⁺ release modulators have been important to understand the roles of Ca²⁺ release from the SR. In the mouse EDL muscle 20 μM dantrolene (a muscle relaxant that inhibits Ca²⁺ release via RyR receptors from the SR) decreased early tetanic tension yet abolished overall fatigue development (E. Germinario et al. J Appl Physiol 96:645-649, 2004). Conversely, caffeine (which stimulates SR Ca²⁺ release and increases free intracellular Ca²⁺ concentrations) increased initial tetanic EDL tension but accelerated and accentuated muscle fatigue to repeated 60 Hz stimulation over 6 minutes (Germinario 2004). In contrast to the changes elicited by dantrolene and caffeine on fatigue where the initial level of tetanic tension caused by the compounds was inversely related to subsequent muscle fatigability, fast skeletal troponin activators as described herein increase initial (submaximal) tension and also enhance fatigue resistance.

Another important aspect of Ca²⁺ release from the SR is the potential to activate Ca²⁺/calmodulin-dependent skeletal muscle myosin light chain kinase (skMLCK) which subsequently phosphorylates the regulatory light chain (RLC) of sarcomeric myosin (H. L. Sweeney et al. Am J Physiol Cell Physiol 264:C1085-C1095, 1993; J. T. Stull et al. Arch Biochem Biophys. 2011 Jun. 15; 510(2): 120-128). Phosphorylation of myosin cross-bridge heads moves the myosin heads away from the thick filament (Sweeney 1993) and enhances thin filament-regulated ATPase activity to increase Ca²⁺ sensitivity toward force generation and rate of force development (D. T. Szczesna et al. J Appl Physiol 92:1661-1670, 2002). Repetitive stimulation of intact muscle and thereby RLC phosphorylation, enhances muscle work and power. The history-dependent force output instills a muscle “memory” when striated muscle has been recently active. Along with metabolism-induced changes in cross bridge function, RLC phosphorylation mechanistically increases the Ca²⁺ sensitivity of the sarcomere to enhance muscle force, work and power, particularly during fatigue. Studies on sarcomere function indicate that RLC increases force responses at submaximal, but not maximal Ca²⁺ activation to shift the force-pCa response to the left (H. L. Sweeney et al. Am J Physiol Cell Physiol 250:C657-C660, 1986; Stull 2011). RLC appears to increase the number of cross-bridges capable of cycling against the thin filament rather than increasing the force per cross-bridge during cycling. In this way, the profile of RLC phosphorylation is similar to the profile defined by fast skeletal troponin activators as described herein. That is, both RLC phosphorylation and fast skeletal troponin activators appear to increase the number of cycling cross-bridges, left-shift the force-pCa response, increase the rate of force development and slow the rate of relaxation of skinned skeletal muscle fibers. RLC phosphorylation may enhance force, work and power generation during submaximal contractions in vivo (Stull 2011), an effect that has also been demonstrated for fast skeletal troponin activators in the current work and in previous studies (Russell A J et al., Nature Medicine, 2011;378:667-75). Both RLC phosphorylation and fast skeletal troponin activators each might enhance low frequency muscle force/power to help preserve muscle function under fatiguing conditions.

Most of the energy utilized by myocytes can be accounted for by two ATP-dependent processes, cross-bridge cycling and Ca²⁺ cycling across cellular membranes. Under resting conditions, SERCA1 is responsible for maintaining a low (<100 nM) cytosolic free Ca²⁺ concentration. In mouse EDL muscle, ATP consumption by SERCAs is responsible for approximately 50% of resting metabolic rate (S. M. Norris et al. Am J Physiol Cell Physiol 298:C521-0529, 2010). Fast skeletal troponin activators reduce the Ca²⁺ requirement in muscle for tension generation, i.e. the same amount of force can be generated with less Ca²⁺ (Russell 2011). A fast skeletal troponin activator's diminished requirement for free cytosolic Ca²⁺ to generate force portends a profound diminution in myocyte SERCA ATP consumption, resulting in substantial energetic savings and improved resistance of muscle to fatigue. This reduction in Ca²⁺ requirement for force generation may play a part in the improved resistance of muscle to fatigue due to fast skeletal troponin activators.

In fatigue, the complicated program of molecular events often acts synergistically to diminish Ca²⁺-activated sarcomere contraction (Debold 2006). Thus, repetitive muscle fiber stimulation leads to impaired coupling of T tubular depolarization to impair proper SR Ca²⁺ release, diminished SR Ca²⁺ content (perhaps due to precipitation with Pi), reduced Ca²⁺ release from SR due to negative metabolic effectors such as ADP, Pi, Mg²⁺ and H⁺, decreased troponin sensitivity to Ca²⁺ resulting from increased levels of Pi, H⁺) and ROS (Westerblad 1991; Allen 2008) and phosphorylation of RyR1 (S. Gehlert et al. PLoS One. 2012; 7(11):e49326), and decreased tension-generating capacity of contractile elements (Debold 2006). Calcium ion release from isolated SR may be reduced by as much as 40% following prolonged or intense exercise in humans (Allen 2008). The increase in sarcomere Ca²⁺ sensitivity elicited by fast skeletal troponin activators addresses these important aspects of Ca²⁺ handling in muscle fatigue, especially those of diminished Ca²⁺ sensitivity resulting from repeated contractions (Westerblad 1991). By normalizing Ca²⁺ sensitivity in contracting muscle, fast skeletal troponin activators decrease fatigability of muscle in vitro, in situ and in vivo. Thus, fast skeletal troponin activators are a useful therapeutic approach to enhance muscle function under conditions of neuromuscular weakness and to alleviate fatigue.

During muscle contraction, the force-velocity relationship is hyperbolic where greater loads produce slower speeds but greater tension. The effect of strength training is to increase the maximum isometric tension (Pₒ) of muscle. Although exercise does not increase the maximum unloaded velocity (Vₒ), a stronger trained muscle can move a load isotonically at a greater velocity. Another aspect of the profiles elicited by fast skeletal troponin activators is the similarity to exercise training. Thus, resistance training in older men led to a 45% increase in Vₒ in type I fibers of the vastus lateralis and an increase in power from 25.5 to 41.1 uN*fiber length/sec (Trappe et al. J. Appl. Physiol. 89:143-152, 2000). In the vastus lateralis type I fibers, the force-velocity relationship was moved upward following exercise, similar to the force-velocity curves described in the current rat studies following fast skeletal troponin activators treatment. Likewise, exercise training progressively increased power output in those subjects (Trappe 2000) in a way reminiscent of the power increases described with fast skeletal troponin activator treatment. Exercise training has been related to improvements in Ca²⁺ handling in skeletal muscle (Ferreira Exp Biol Med 235:497-505, 2010) and exercise performance (G. C. Bogdanis. Frontiers in Physiol. May 2012, Vol. 3, Article 142, pp. 1-15).

It is notable that conditions that left-shift the force-pCa curve such as skeletal muscle RLC phosphorylation, exercise training or fast skeletal troponin activator binding to the troponin complex all improve muscle fatigability. The similarities in muscle performance due to these three conditions underscore the importance of sarcomeric Ca²⁺ sensitivity in muscle performance and fatigue resistance. Unlike attempts to enhance muscle performance that involve AMPK activation (e.g. exercise training or treatment with the small molecule AlCAR) which require long-term alterations in gene expression of numerous targets, fast skeletal troponin activators such as those described herein rapidly (i.e., within minutes to hours) enhance sub-maximal muscle force development and fatigability and are specific for the fast skeletal troponin complex. This enables a selective pharmacological therapy to enhance skeletal muscle function for a variety of potential disease states, especially those where fatigue is a cardinal symptom.

Development of muscle fatigue is often described as a decline in maximal force or power capacity of muscle, implying that submaximal contractions are sustainable following the onset of muscle fatigue. Muscle fatigue might describe a reduction in muscle force capacity, decreased endpoints for a sustained activity, exhaustion of contractile function or possibly a waning in mental function (R. M. Enoka and J. Duchateau. J Physiol 586.1 (2008) pp 11-23). The mechanism(s) involved in fatigue depend on the task being performed and must include both the perception of fatigue and the mechanisms that define muscle fatigability (Enoka et al. J. Biomech. 45:427-433, 2012). Not only are there different fatigue mechanisms at play in isometric vs. isotonic muscle contraction, but even different protocols for isometric contractions can yield different fatigue profiles. Thus, in human volunteers asked to either push their arm up against a force transducer to maintain submaximal target force or to perform an identically-matched net muscle torque task of supporting an inertial load with the elbow flexor muscles, the fatigue profile was dramatically different (S. K. Hunter et al. J Neurophysiol 188:3087-3096, 2002). The endurance time for maintaining the force task (1402 sec) was twice as long as for the position task (702 sec) and had a lower level of excitatory and inhibitory input to the motor neurons compared to the position task in those studies. Fatigue-related adjustments in motor unit recruitment appears, thus, to also influence the sensations associated with fatiguing contractions, for example, there is a strong association between fatigability and the perception of exertion (Enoka and Duchateau, 2008). It is therefore important to not only define the potential effects of fast skeletal troponin activators on muscle function in vitro or in unconscious animal models, but to also explore effects on exercise capacity and fatigue in conscious animal models of static and dynamic exercise. The effects of fast skeletal troponin activators can be evaluated in dynamic exercise models in laboratory animals, such as an accelerating rotarod assay (O. Fanellie. Pharmacology. 1976;14(1):52-7; N. Boyadjiev et al. Journal of Sports Science and Medicine (2007) 6, 423-428), a treadmill endurance-type fatigue assay, or a cage grid hang time assay, as described herein. The effects of fast skeletal troponin activators can also be evaluated in dynamic exercise studies using human subjects, for example using a bilateral heel raise test, as described herein.

Provided are methods for improving resistance to skeletal muscle fatigue in a subject in need thereof, said method comprising administering to the subject an effective amount of a skeletal muscle troponin activator. In some embodiments, the skeletal muscle troponin activator is a fast skeletal muscle troponin activator. In some embodiments, the subject is suffering from a condition selected from peripheral artery disease, claudication, and muscle ischemia.

Also provided are methods of improving resistance to fatigue in a skeletal muscle, comprising contacting the skeletal muscle with a skeletal muscle troponin activator, wherein the skeletal muscle troponin activator increases submaximal tension in the skeletal muscle.

Also provided are methods of improving resistance to fatigue in a skeletal muscle, comprising contacting the skeletal muscle with a skeletal muscle troponin activator, wherein the skeletal muscle troponin activator reduces the calcium required by the skeletal muscle to generate force.

Also provided are methods of improving resistance to fatigue in a skeletal muscle, comprising contacting the skeletal muscle with a skeletal muscle troponin activator, wherein the skeletal muscle troponin activator increases the rate of force development in the skeletal muscle.

In some of embodiments disclosed herein, the skeletal muscle troponin activator is a fast skeletal muscle troponin activator.

In some embodiments, the improvement in resistance to skeletal muscle fatigue in the subject is determined by a bilateral heel-raise test as described herein (see Examples 10 and 11 herein). In some embodiments, the bilateral heel-raise test comprises instructing the subject to perform heel raises at regular intervals and measuring the value of one or more parameters selected from (a) time to claudication onset, (b) number of heel raises to claudication onset, (c) work to claudication onset, (d) time to maximal claudication fatigue, (e) number of heel raises to maximal claudication fatigue, and (d) work to maximal claudication fatigue, wherein an increase in the one or more of the parameters indicates an improvement in resistance to fatigue in the subject.

In some embodiments, the skeletal muscle troponin activator is a compound of Formula I:

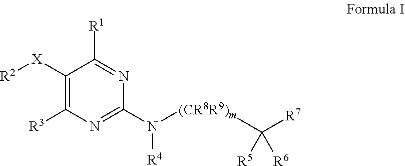

or a pharmaceutically acceptable salt thereof, wherein:

R¹ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;

R² is selected from C₃₋₈ cycloalkyl, C₃₋₈ cycloalkenyl, 3-8 membered heterocycloalkyl, 3-8 membered heterocycloalkenyl, C₆₋₁₀ aryl, 5-10 membered heteroaryl and NR^{b}R^{c}, wherein each of the C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl groups is optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)Rₐ, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl groups is optionally substituted with 1, 2, 3, 4 or 5 R^{f} substituents;

R³ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;

R⁴ is selected from hydrogen, C₁₋₆ alkyl, C₁₋₆ haloalkyl, C(O) R^{a}, C(O)OR^{a}, C(O)NR^{b}R^{c} and SO₂R^{a};

R⁵ and R⁶ are each independently selected from hydrogen, halogen, C₁₋₆ alkyl and C₁₋₆ haloalkyl;

or alternatively, R⁵ and R⁶ together with the carbon atom to which they are bound form a group selected from C₃₋₈ cycloalkyl, C₃₋₈ cycloalkenyl, 3-8 membered heterocycloalkyl and 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl;

R⁷ is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;

R⁸ and R⁹, at each occurrence, are each independently selected from hydrogen, halogen and C₁₋₆ alkyl;

X is selected from a bond, —(CH₂)ₚ—, —(CH₂)ₚC(O)(CH²)_{q}—, —(CH₂)ₚO(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚ0C(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—;

or alternatively, X, R² and R³, together with the carbon atoms to which they are bound, form a 5-6 membered ring optionally containing one or more heteroatoms selected from oxygen nitrogen and sulfur, and optionally containing one or more double bonds, and optionally substituted with 1, 2, 3, 4 or 5 R^{f} substituents;

R^{a}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₈₋₁₀ aryl, C₇₋₁₁ aralkyl and 5-10 membered heteroaryl groups is optionally substituted with 1, 2, 3, 4 or 5 R^{f} substituents;

R^{b} and R^{c}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, C(O)OR^{g}, C(O)NRⁱR^{j} and SO₂R^{g}, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;

R^{d}, at each occurrence, is independently selected from hydrogen and C₁₋₆ alkyl;

R^{e}, at each occurrence, is independently selected from hydrogen, CN, OH, C₁₋₆ alkoxy, C₁₋₆ alkyl and C₁₋₆ haloalkyl;

R^{f}, at each occurrence, is independently selected from halogen, CN, OR^{h}, OC(O)R^{h}, OC(O)OR^{h}, OC(O)NRⁱR^{j}, NRⁱR^{j}, NR^{d}C(O)R^{h}, NR^{d}C(O)OR^{h}, NR^{d}C(O)NRⁱR^{j}, NR^{d}C(O)C(O)NRⁱR^{j}, NR^{d}C(S)R^{h}, NR^{d}C(S)OR^{h}, NR^{d}C(S)NRⁱR^{j}, NR^{d}C(NR^{e})NRⁱR^{j}, NR^{d}S(O)R^{h}, NR^{d}SO₂R^{h}, NR^{d}SO₂NRⁱR^{j}, C(O)R^{h}, C(O)OR^{h}, C(O)NRⁱR^{j}, C(S)R^{h}, C(S)OR^{h}, C(S)NRⁱR^{j}, C(NR^{e})NRⁱR^{j}, SR^{h}, S(O)R^{h}, SO₂R^{h}, SO₂NRⁱR^{j}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;

or two R^{f} substituents bound to a single carbon atom, together with the carbon atom to which they are both bound, form a group selected from carbonyl, C₃₋₈ cycloalkyl and 3-8 membered heterocycloalkyl;

R^{g}, at each occurrence, is independently selected from C₁₋₆ alkyl, C₁₋₆ haloalkyl, phenyl, naphthyl, and C₇₋₁₁ aralkyl, each optionally substituted with 1, 2, 3, 4 or 5 substituents selected from halogen, CN, OH, C₁₋₆ alkoxy, C₁₋₆ alkyl and C₁₋₆ haloalkyl;

R^{h}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;

Rⁱ and R^{j}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, and C(O)OR^{g}, wherein each of the C₁₋₆ alkyl, C₁₋₆ haloalkyl, C₂₋₆ alkenyl, C₂₋₆ alkynyl, C₃₋₈ cycloalkyl, 3-8 cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 substituents selected from halogen, CN, OH, C₁₋₆ alkoxy, C₁₋₆ alkyl and C₁₋₆ haloalkyl;

R^{k}, at each occurrence, is independently selected from halogen, CN, OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, NHC(O)OC₇₋₁₁ aralkyl, OC(O)C₁₋₆ alkyl, OC(O)C₇₋₁₁ aralkyl, OC(O)OC₁₋₆ alkyl, OC(O)OC₇₋₁₁ aralkyl, C(O)C₁₋₆ alkyl, C(O)C₇₋₁₁ aralkyl, C(O)OC₁₋₆ alkyl, C(O)OC₇₋₁₁ aralkyl, C₁₋₆ alkyl, C₁₋₆ haloalkyl, C₂₋₆ alkenyl, and C₂₋₆ alkynyl, wherein each C₁₋₆ alkyl, C₂₋₆ alkenyl, C₂₋₆ alkynyl, and C₇₋₁₁ aralkyl substituent is optionally substituted with 1, 2 or 3 substituents selected from OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, and NHC(O)OC₇₋₁₁ aralkyl;

or two R^{k} substituents bound to a single carbon atom, together with the carbon atom to which they are both bound, form a carbonyl group;

m is 0, 1 or 2;

n, at each occurrence, independently is 0, 1 or 2;

p is 0, 1 or 2; and

q is 0, 1 or 2.

In some embodiments of compounds of Formula I, m is 0, i.e., a compound of Formula II, or a pharmaceutically acceptable salt thereof:

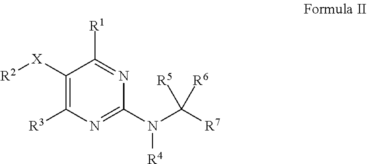

wherein R¹, R², R³, R⁴, R⁵, R⁶, R⁷ and X are as defined herein.

In some embodiments of compounds of Formula I, m is 1, i.e., a compound of Formula III, or a pharmaceutically acceptable salt thereof:

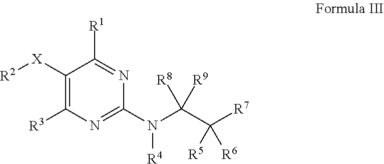

wherein R¹, R², R³, R⁴, R⁵, R⁶, R⁷, R⁸, R⁹ and X are as defined herein.

In some embodiments of compounds of Formula I, II or III, one of R⁵ and R⁶ is hydrogen and the other is C₁₋₆ alkyl.

In some embodiments of compounds of Formula I, II or III, R⁵ and R⁶ are each independently C₁₋₆ alkyl.

In some embodiments of compounds of Formula I, II or III, R⁵ and R⁶ are each methyl.

In some embodiments, the compounds are of Formula IV(a) or IV(b), or a pharmaceutically acceptable salt thereof:

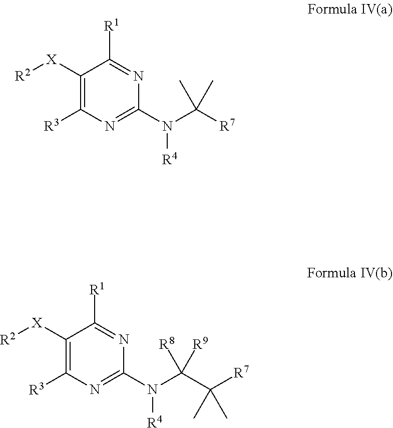

wherein R¹, R², R³, R⁴, R⁷, R⁸, R⁹ and X are as defined herein.

In some embodiments of compounds of Formula I, II or III, R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶, together with the carbon to which they are bound, form C₃₋₆ cycloalkyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶, together with the carbon to which they are bound, form cyclopropyl, cyclobutyl, cyclopentyl or cyclohexyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶, together with the carbon to which they are bound, form cyclobutyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶, together with the carbon to which they are bound, form cyclobutyl substituted with one substituent selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl, wherein the substituent and R⁷ are in a trans configuration with respect to one another on the cyclobutyl ring.

In some embodiments of compounds of Formula I, II or III, R⁵ and R⁶, together with the carbon to which they are bound, form cyclobutyl substituted with one substituent selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl, wherein the substituent and R⁷ are in a cis configuration with respect to one another on the cyclobutyl ring.

In some embodiments, the compounds are of Formula V(a) or V(b), or a pharmaceutically acceptable salt thereof:

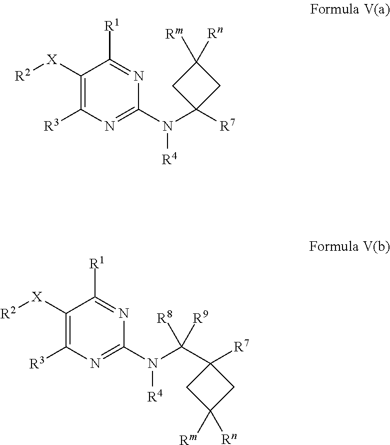

wherein R^{m} and Rⁿ are each independently selected from hydrogen, halogen and C₁₋₆ alkyl, and R¹, R², R³, R⁴, R⁷, R⁸, R⁹ and X are as defined herein.

In some embodiments of compounds of Formula V(a) or V(b), R^{m} and Rⁿ are each hydrogen.

In some embodiments compounds of Formula V(a) or V(b), R^{m} and Rⁿ are each halogen.

In some embodiments compounds of Formula V(a) or V(b), R^{m} and Rⁿ are each fluorine.

In some embodiments compounds of Formula V(a) or V(b), one of R^{m} and Rⁿ is hydrogen and the other is halogen. In some embodiments of such compounds, the halogen and R⁷ are in a trans configuration with respect to one another on the cyclobutyl ring. In some embodiments of such compounds, the halogen and R⁷ are in a cis configuration with respect to one another on the cyclobutyl ring.

In some embodiments compounds of Formula V(a) or V(b), one of R^{m} and Rⁿ is hydrogen and the other is fluorine. In some embodiments of such compounds, the fluorine and R⁷ are in a trans configuration with respect to one another on the cyclobutyl ring. In some embodiments of such compounds, the fluorine and R⁷ are in a cis configuration with respect to one another on the cyclobutyl ring.

In some embodiments of compounds of Formula I, II or III, R⁵ and R⁶, together with the carbon atom to which they are bound, form 3-6 membered heterocycloalkyl, each of which is optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶, together with the carbon atom to which they are bound, form aziridine, azetidine, pyrrolidine, oxirane, oxetane or tetrahydrofuran, each of which is optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶ are each independently C₁₋₆ alkyl, or R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶ are each methyl, or R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶ are each independently C₁₋₆ alkyl, or R⁵ and R⁶, together with the carbon to which they are bound, form cyclopropyl, cyclobutyl, cyclopentyl or cyclohexyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II or III, R⁵ and R⁶ are each methyl, or R⁵ and R⁶, together with the carbon to which they are bound, form cyclobutyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments of compounds of Formula I, II, III, IV(a), IV(b), V(a) or V(b), R⁷ is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, 0₃-₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R¹ substituents.

In some embodiments of compounds of Formula I, II, III, IV(a), IV(b), V(a) or V(b), R⁷ is phenyl optionally substituted with 1, 2, 3, 4 or 5 substituents selected from halogen, CN, oxo, OR^{a}, OC(O)R^{a}, OC(O)OR^{a}, OC(0)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)0R^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, the compounds are of Formula VI, or a pharmaceutically acceptable salt thereof:

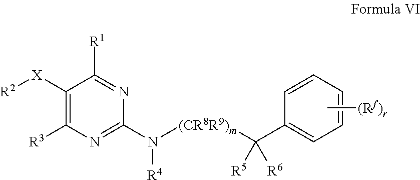

wherein r is 0, 1, 2, 3 or 4, and R¹, R², R³, R⁴, R⁵, R⁶, R⁸, R⁹, R^{f}, X and m are as defined herein.

In some embodiments, the compounds are of Formula VII(a) or VII(b), or a pharmaceutically acceptable salt thereof:

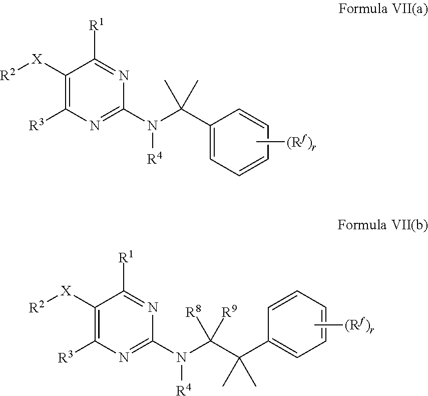

wherein r is 0, 1, 2, 3 or 4, and R¹, R², R³, R⁴, R⁸, R⁹, R^{f} and X are as defined herein.

In some embodiments, the compounds are of Formula VIII(a) or VIII(b), or a pharmaceutically acceptable salt thereof:

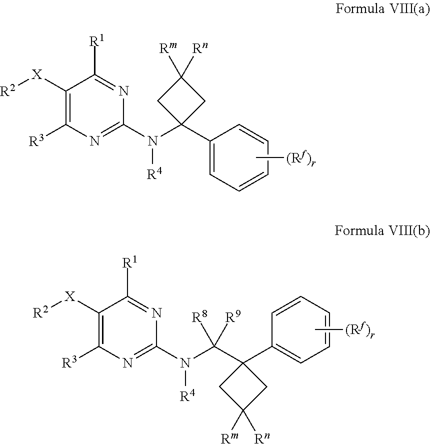

wherein R^{m} and Rⁿ are each independently selected from hydrogen, halogen and C₁₋₆ alkyl; r is 0, 1, 2, 3 or 4; and R¹, R², R³, R⁴, R⁸, R⁹, R^{f} and X are as defined herein.

In some embodiments of compounds of Formula VIII(a) or VIII(b), R^{m} and Rⁿ are each hydrogen.

In some embodiments compounds of Formula VIII(a) or VIII(b), R^{m} and Rⁿ are each halogen.

In some embodiments compounds of Formula VIII(a) or VIII(b), R^{m} and Rⁿ are each fluorine.

In some embodiments compounds of Formula VIII(a) or VIII(b), one of R^{m} and Rⁿ is hydrogen and the other is halogen. In some embodiments of such compounds, the halogen and the phenyl ring are in a trans configuration with respect to one another on the cyclobutyl ring. In some embodiments of such compounds, the halogen and the phenyl ring are in a cis configuration with respect to one another on the cyclobutyl ring.

In some embodiments compounds of Formula VIII(a) or VIII(b), one of R^{m} and Rⁿ is hydrogen and the other is fluorine. In some embodiments of such compounds, the fluorine and the phenyl ring are in a trans configuration with respect to one another on the cyclobutyl ring. In some embodiments of such compounds, the fluorine and the phenyl ring are in a cis configuration with respect to one another on the cyclobutyl ring.

In some embodiments of compounds of Formula I, II, III, IV(a), IV(b), V(a) or V(b), R⁷ is selected from phenyl, 2-fluorophenyl, 3-fluorophenyl, 2,4-difluorophenyl, 3,4-difluorophenyl, 3,5-difluorophenyl, 4-fluorophenyl, 2-chlorophenyl, 3-chlorophenyl, 4-chlorophenyl, 2,4-dichlorophenyl, 3,4-dichlorophenyl, 3,5-dichlorophenyl, 2-methylphenyl, 3-methylphenyl, 2,4-dimethylphenyl, 3,4-dimethylphenyl, 3,5-dimethylphenyl, 2-(hydroxymethyl)phenyl, 3-(hydroxymethyl)phenyl, 4-(hydroxymethyl)phenyl, 2-(aminomethyl)phenyl, 3-(aminomethyl)phenyl, 4-(aminomethyl)phenyl, 2-phenol, 3-phenol, 4-phenol, 2-methoxyphenyl, 3-methoxyphenyl, 4-methoxyphenyl, 2-difluoromethoxyphenyl, 3-difluoromethoxyphenyl, 4-difluoromethoxyphenyl, 2-trifluoromethoxyphenyl, 3-trifluoromethoxyphenyl, 4-trifluoromethoxyphenyl, 2-cyanophenyl, 3-cyanophenyl, 4-cyanophenyl, 2-benzamine, 3-benzamide, 4-benzamide, N-methyl-2-benzamine, N-methyl-3-benzamide, N-methyl-4-benzamide, N,N-dimethyl-2-benzamine, N,N-dimethyl-3-benzamide, and N,N-dimethyl-4-benzamide.

In some embodiments of compounds of Formula I, II, III, IV(a), IV(b), V(a) or V(b), R⁷ is 5-10 membered heteroaryl optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂-₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments of compounds of Formula I, II, III, IV(a), IV(b), V(a) or V(b), R⁷ is pyridyl optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments of compounds of Formula I, II, III, IV(a), IV(b), V(a) or V(b), R⁷ is selected from 2-pyridyl, 3-pyridyl and 4-pyridyl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₆ cycloalkyl, C₃₋₆ cycloalkenyl, 3-6 membered heterocycloalkyl, 3-6 membered heterocycloalkenyl, phenyl, naphthyl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, the compounds are of Formula IX, or a pharmaceutically acceptable salt thereof:

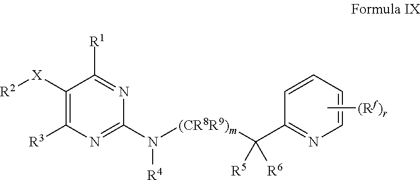

wherein r is 0, 1, 2, 3 or 4, and R¹, R², R³, R⁴, R⁵, R⁶, R⁸, R⁹, R^{f}, X and m are as defined herein.

In some embodiments, the compounds are of Formula X(a) or X(b), or a pharmaceutically acceptable salt thereof:

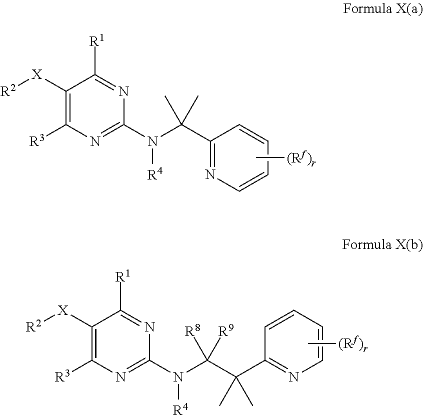

wherein r is 0, 1, 2, 3 or 4, and R¹, R², R³, R⁴, R⁸, R⁹, R^{f} and X are as defined herein.

In some embodiments, the compounds are of Formula XI(a) or XI(b), or a pharmaceutically acceptable salt thereof:

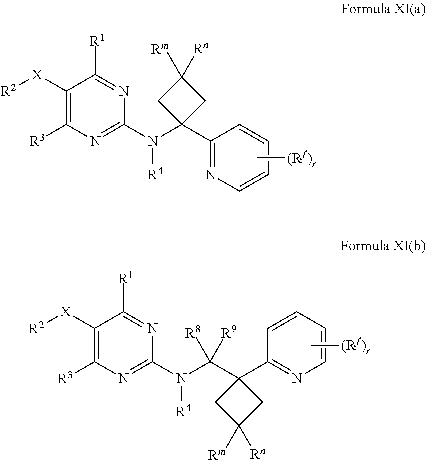

wherein R^{m} and Rⁿ are each independently selected from hydrogen, halogen and C₁₋₆ alkyl; r is 0, 1, 2, 3 or 4; and R¹, R², R³, R⁴, R⁸, R⁹, R^{f} and X are as defined herein.

In some embodiments of compounds of Formula XI(a) or XI(b), R^{m} and Rⁿ are each hydrogen.

In some embodiments compounds of Formula XI(a) or XI(b), R^{m} and Rⁿ are each halogen.

In some embodiments compounds of Formula XI(a) or XI(b), R^{m} and Rⁿ are each fluorine.

In some embodiments compounds of Formula XI(a) or XI(b), one of R^{m} and Rⁿ is hydrogen and the other is halogen. In some embodiments of such compounds, the halogen and the pyridyl ring are in a trans configuration with respect to one another on the cyclobutyl ring. In some embodiments of such compounds, the halogen and the pyridyl ring are in a cis configuration with respect to one another on the cyclobutyl ring.

In some embodiments compounds of Formula XI(a) or XI(b), one of R^{m} and Rⁿ is hydrogen and the other is fluorine. In some embodiments of such compounds, the fluorine and the pyridyl ring are in a trans configuration with respect to one another on the cyclobutyl ring. In some embodiments of such compounds, the fluorine and the pyridyl ring are in a cis configuration with respect to one another on the cyclobutyl ring.

In some embodiments of compounds of Formula I, II, III, IV(a), IV(b), V(a) or V(b), R⁷ is selected from pyrid-2-yl, 3-fluoro-pyrid-2-yl, 4-fluoro-pyrid-2-yl, 5-fluoro-pyrid-2-yl, 6-fluoro-pyrid-2-yl, 3-chloro-pyrid-2-yl, 4-chloro-pyrid-2-yl, 5-chloro-pyrid-2-yl, 6-chloro-pyrid-2-yl, 3-cyano-pyrid-2-yl, 4-cyano-pyrid-2-yl, 5-cyano-pyrid-2-yl, 6-cyano-pyrid-2-yl, 3-methyl-pyrid-2-yl, 4-methyl-pyrid-2-yl, 5-methyl-pyrid-2-yl, 6-methyl-pyrid-2-yl, 3-difluoromethyl-pyrid-2-yl, 4-difluoromethyl-pyrid-2-yl, 5-difluoromethyl-pyrid-2-yl, 6-difluoromethyl-pyrid-2-yl, 3-trifluoromethyl-pyrid-2-yl, 4-trifluoromethyl-pyrid-2-yl, 5-trifluoromethyl-pyrid-2-yl, 6-trifluoromethyl-pyrid-2-yl, 3-hydroxymethyl-pyrid-2-yl, 4-hydroxymethyl-pyrid-2-yl, 5-hydroxymethyl-pyrid-2-yl, 6-hydroxymethyl-pyrid-2-yl, 3-aminomethyl-pyrid-2-yl, 4-aminomethyl-pyrid-2-yl, 5-aminomethyl-pyrid-2-yl, 6-aminomethyl-pyrid-2-yl, 3-hydroxy-pyrid-2-yl, 4-hydroxy-pyrid-2-yl, 5-hydroxy-pyrid-2-yl, 6-hydroxy-pyrid-2-yl, 3-methoxy-pyrid-2-yl, 4-methoxy-pyrid-2-yl, 5-methoxy-pyrid-2-yl, 6-methoxy-pyrid-2-yl, 3-difluoromethoxy-pyrid-2-yl, 4-difluoromethoxy-pyrid-2-yl, 5-difluoromethoxy-pyrid-2-yl, 6-difluoromethoxy-pyrid-2-yl, 3-trifluoromethoxy-pyrid-2-yl, 4-trifluoromethoxy-pyrid-2-yl, 5-trifluoromethoxy-pyrid-2-yl, 6-trifluoromethoxy-pyrid-2-yl, 3-methylthio-pyrid-2-yl, 4-methylthio-pyrid-2-yl, 5-methylthio-pyrid-2-yl, 6-methylthio-pyrid-2-yl, 3-carboxamide-pyrid-2-yl, 4-carboxamide-pyrid-2-yl, 5-carboxamide-pyrid-2-yl, 6-carboxamide-pyrid-2-yl and 3-fluoro-6-methyl-pyrid-2-yl.

In some embodiments of compounds of Formula I, II, III, IV(a), IV(b), V(a) or V(b), R⁷ is selected from pyrid-3-yl, 2-fluoro-pyrid-3-yl, 4-fluoro-pyrid-3-yl, 5-fluoro-pyrid-3-yl, 6-fluoro-pyrid-3-yl, 2-chloro-pyrid-3-yl, 4-chloro-pyrid-3-yl, 5-chloro-pyrid-3-yl, 6-chloro-pyrid-3-yl, 2-cyano-pyrid-3-yl, 4-cyano-pyrid-3-yl, 5-cyano-pyrid-3-yl, 6-cyano-pyrid-3-yl, 2-methyl-pyrid-3-yl, 4-methyl-pyrid-3-yl, 5-methyl-pyrid-3-yl, 6-methyl-pyrid-3-yl, 2-difluoromethyl-pyrid-3-yl, 4-difluoromethyl-pyrid-3-yl, 5-difluoromethyl-pyrid-3-yl, 6-difluoromethyl-pyrid-3-yl, 2-trifluoromethyl-pyrid-3-yl, 4-trifluoromethyl-pyrid-3-yl, 5-trifluoromethyl-pyrid-3-yl, 6-trifluoromethyl-pyrid-3-yl, 2-hydroxymethyl-pyrid-3-yl, 4-hydroxymethyl-pyrid-3-yl, 5-hydroxymethyl-pyrid-3-yl, 6-hydroxymethyl-pyrid-3-yl, 2-aminomethyl-pyrid-3-yl, 4-aminomethyl-pyrid-3-yl, 5-aminomethyl-pyrid-3-yl, 6-aminomethyl-pyrid-3-yl, 2-hydroxy-pyrid-3-yl, 4-hydroxy-pyrid-3-yl, 5-hydroxy-pyrid-3-yl, 6-hydroxy-pyrid-3-yl, 2-methoxy-pyrid-3-yl, 4-methoxy-pyrid-3-yl, 5-methoxy-pyrid-3-yl, 6-methoxy-pyrid-3-yl, 2-difluoromethoxy-pyrid-3-yl, 4-difluoromethoxy-pyrid-3-yl, 5-difluoromethoxy-pyrid-3-yl, 6-difluoromethoxy-pyrid-3-yl, 2-trifluoromethoxy-pyrid-3-yl, 4-trifluoromethoxy-pyrid-3-yl, 5-trifluoromethoxy-pyrid-3-yl, 6-trifluoromethoxy-pyrid-3-yl, 2-methylthio-pyrid-3-yl, 4-methylthio-pyrid-3-yl, 5-methylthio-pyrid-3-yl, 6-methylthio-pyrid-3-yl, 2-carboxamide-pyrid-3-yl, 4-carboxamide-pyrid-3-yl, 5-carboxamide-pyrid-3-yl and 6-carboxamide-pyrid-3-yl.

In some embodiments of compounds of Formula I, II, III, IV(a), IV(b), V(a), V(b), VI, VII(a), VII(b), VIII(a), VIII(b), IX, X(a), X(b), XI(a) or XI(b), X is selected from a bond, —(CH₂)ₚ—, —(CH₂)ₚO(CH₂)_{q}—, —(CH₂)ₚC(O)(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚOC(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is a bond.

In some embodiments, the compound is of Formula XII(a), XII(b), XII(c), XII(d), XII(e), XII(f), XII(g), XII(h), XII(i), XII(j), XII(k), XII(l), XII(m), XII(n) or XII(o), or a pharmaceutically acceptable salt thereof:

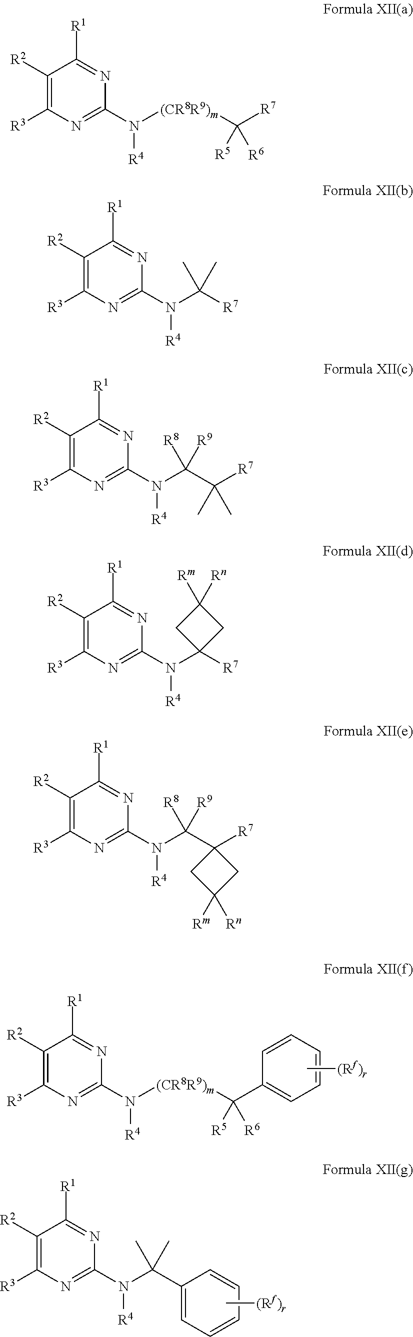

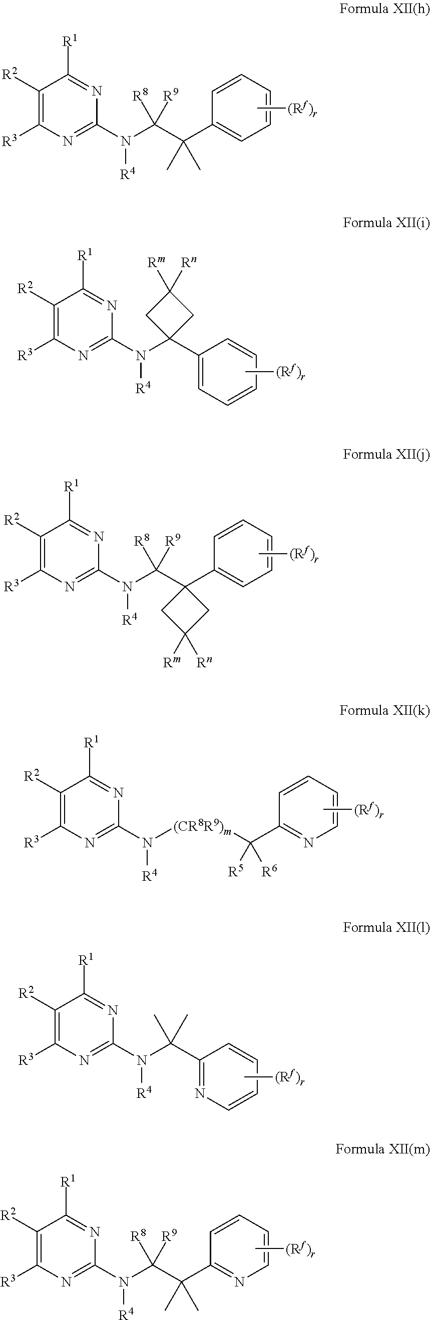

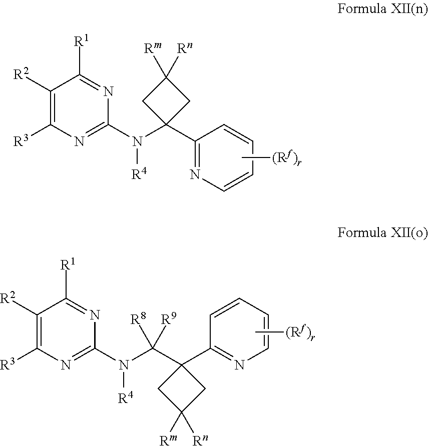

wherein R¹, R², R³, R⁴, R⁵, R⁶, R⁷, R⁸, R⁹, R^{f}, R^{m}, Rⁿ, m and r are as defined herein.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —O—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is selected from —CH₂O— and —OCH₂—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —NR^{d}—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is selected from —CH₂NR^{d}— and —NR^{d}CH₂—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is selected from —NR^{d}C(O)— and —C(O)NR^{d}—.

In some embodiments of compounds of Formula I, II, III, IV(a), IV(b), V(a), V(b), VI, VII(a), VII(b), VIII(a), VIII(b), IX, X(a), X(b), XI(a) or XI(b), X is selected from —CH₂NR^{d}C(O)— and —C(O)NR^{d}CH₂—.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl optionally substituted with 1, 2, 3, 4 or 5 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted with 1, 2, 3, 4 or 5 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents; wherein at least one substitutent is bonded at the meta position.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted with a substituent selected from (CH₂)ₙC(O)OR^{a} and (CH₂)ₙC(O)NR^{b}R^{c}; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b})R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted with a substituent selected from C(O)OH, C(O)NH₂, C(O)OC₁₋₆ alkyl, C(O)NHC,₁₋₆ alkyl and C(O)N(C₁₋₆ alkyl)₂; and optionally substituted with 1, 2 or 3 additional substituents selected from halogen, C₁₋₆ alkyl and C₁₋₆ haloalkyl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted at the meta position with a substituent selected from (CH₂)ₙC(O)OR^{a} and (CH₂)ₙC(O)NR^{b}R^{c}; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted at the meta position with a substituent selected from (CH₂)ₙC(O)OR^{a} and (CH₂)ₙC(O)NR^{b}R^{c}, and optionally substituted with 1, 2 or 3 additional substituents selected from halogen, hydroxyl, C₁₋₆ alkoxy, CN, C₁₋₆ alkyl and C₁₋₆ haloalkyl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted at the meta position with a substituent selected from C(O)OH, C(O)NH₂, C(O)OC₁₋₆ alkyl, C(O)NHC₁₋₆ alkyl and C(O)N(C₁₋₆ alkyl)₂; and optionally substituted with 1, 2 or 3 additional substituents selected from halogen, hydroxyl, C₁₋₆ alkoxy, CN, C₁₋₆ alkyl and C₁₋₆ haloalkyl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted with (CH₂)ₙNR^{d}C(O)R^{a}, wherein R^{a} is C₁₋₆ alkyl or 3-8 membered heterocycloalkyl, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is phenyl substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, and (CH₂)ₙ5-10 membered heteroaryl; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is 3-benzamide, N-methyl-3-benzamide, N,N-dimethyl-3-benzamide, 4-fluoro-3-benzamide, N-methyl-4-fluoro-3-benzamide, N,N-dimethyl-4-fluoro-3-benzamide, 3-benzoic acid, methyl-3-benzoate, 4-fluoro-3-benzoic acid and methyl-4-fluoro-3-benzoate.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is 5-10 membered heteroaryl optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with 1, 2, 3 or 4 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with a substituent selected from (CH₂)ₙC(O)OR^{a} and (CH₂)ₙC(O)NR^{b}R^{c}; and optionally substituted with 1, 2 or 3 additional substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl and triazyl, each optionally substituted with (CH₂)ₙC(O)NR^{b}R^{c}.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙC(O)NR^{b}R^{c}.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl and triazyl, each optionally substituted with (CH₂)ₙC(O)NH₂.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙC(O)NH₂.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙNR^{d}C(O)R^{a}, wherein R^{a} is C₁₋₆ alkyl or 3-8 membered heterocycloalkyl, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₅ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from pyridyl, pyrimidyl, pyrazyl, pyridazyl and triazyl, each optionally substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from indolyl, indazolyl, benzimidazolyl, benzoxazolyl and benzoisoxazolyl, each optionally substituted with 1, 2, 3 or 4 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from 1H-indazol-6-yl, 1H-indazol-5-yl, 1H-indazol-4-yl, 3-amino(1H-indazol-5-yl), 3-amino(1H-indazol-6-yl), 3-amino(1H-indazol-7-yl), 1-methyl(1H-indazol-6-yl), 3-methyl(1H-indazol-6-yl), 3-amino-1-methyl(1H-indazol-5-yl), 3-cyano(1H-indazol-5-yl), 3-carboxamide(1H-indazol-5-yl), 3-carboxamidine(1H-indazol-5-yl), 3-vinyl(1H-indazol-5-yl), 3-ethyl(1H-indazol-5-yl), 3-acetamide(1H-indazol-5-yl), 3-methylsulfonylamine(1H-indazol-5-yl), 3-methoxycarboxamide(1H-indazol-5-yl), 3-methylamino(1H-indazol-5-yl), 3-dimethylamino(1H-indazol-5-yl), 3-ethylamino(1H-indazol-5-yl), 3-(2-aminoethyl)amino(1H-indazol-5-yl), 3-(2-hydroxyethyl)amino(1H-indazol-5-yl), 3-[(methylethyl)amino](1H-indazol-5-yl), 6-benzimidazol-5-yl, 6-(2-methylbenzimidazol-5-yl), 2-aminobenzimidazol-5-yl, 2-hydroxybenzimidazol-5-yl, 2-acetamidebenzimidazol-5-yl, 3-aminobenzo[3,4-d]isoxazol-5-yl, 3-aminobenzo[d]isoxazol-6-yl, 3-aminobenzo[d]isoxazol-7-yl, 2-methylbenzoxazol-5-yl and 2-methylbenzoxazol-6-yl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from 3-6 membered heterocycloalkyl and 3-6 membered heterocycloalkenyl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R² is selected from aziridinyl, azetidinyl, pyrrolidinyl, piperidinyl, piperazinyl and morpholinyl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a) or XI(b), R² is NR^{b}R^{c}, wherein R^{b} and R^{c} are as defined herein.

In some embodiments of compounds of Formula I, II, III, IV(a), IV(b), V(a), V(b), VI, VII(a), VII(b), VIII(a), VIII(b), IX, X(a), X(b), XI(a) or XI(b), R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —C(O)13 and R² is NR^{b}R^{c}, wherein R^{b} and R^{c} are as defined herein.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —C(O)— and R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —(CH₂)ₚ— and R² is NR^{b}R^{c}, wherein R^{b} and R^{c} are as defined herein.

In some embodiments of compounds of Formula I, II, III, IV(a), IV(b), V(a), V(b), VI, VII(a), VII(b), VIII(a), VIII(b), IX, X(a), X(b), XI(a) or XI(b), X is —(CH₂)ₚ— and R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments, X, R² and R³, together with the carbon atoms to which they are bound, form a 5-6 membered ring optionally containing one or more heteroatoms selected from oxygen nitrogen and sulfur, and optionally containing one or more double bonds, and optionally substituted with 1, 2, 3, 4 or 5 R^{f} substituents.

In some embodiments, the compound is of Formula XIII, or a pharmaceutically acceptable salt thereof:

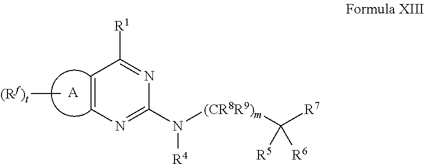

wherein A is a 5 or 6 membered ring optionally containing one or more heteroatoms selected from oxygen nitrogen and sulfur, and optionally containing one or more double bonds; t is 0, 1, 2, 3 or 4; and R¹, R⁴, R⁵, R⁶, R⁷, R⁸, R⁹, R^{f} and m are as defined herein.

In some embodiments of compounds of Formula XIII, ring A together with the pyrimidine ring to which it is bound form a group selected from quinazoline, pyrido[2,3-d]pyrimidine, pyrido[3,4-d]pyrimidine, pyrido[4,3-d]pyrimidine, pyrido[3,2-d]pyrimidine, 5,6,7,8-tetrahydroquinazoline, 5,6,7,8-tetrahydropyrido[2,3-d]pyrimidine, 5,6,7,8-tetrahydropyrido[3,4-d]pyrimidine, 5,6,7,8-tetrahydropyrido[4,3-d]pyrimidine, 5,6,7,8-tetrahydropyrido[3,2-d]pyrimidine, thieno[3,2-d]pyrimidine, thiazolo[4,5-d]pyrimidine, 5H-pyrrolo[3,2-d]pyrimidine, 7H-purine, thieno[2,3-d]pyrimidine, thiazolo[5,4-d]pyrimidine, 7H-pyrrolo[2,3-d]pyrimidine, 9H-purine, 1H-pyrazolo[4,3-d]pyrimidine, 1H-pyrazolo[3,4-d]pyrimidine, 1H-[1,2,3]triazolo[4,5-d]pyrimidine, 3H-[1,2,3]triazolo[4,5-d]pyrimidine, 6,7-dihydro-5H-pyrrolo[2,3-d]pyrimidine, 6,7-dihydro-5H-pyrrolo[3,4-d]pyrimidine, 6,7-dihydro-5H-pyrrolo[3,2-d]pyrimidine and 6,7-dihydro-5H-cyclopenta[d]pyrimidine, each optionally substituted with 1, 2, 3, 4 or 5 R^{f} substituents.

In some embodiments of compounds of Formula XIII, Ring A together with the pyrimidine ring to which it is bound form a group selected from quinazoline, 5,6,7,8-tetrahydropyrido[4,3-d]pyrimidine, 5,6,7,8-tetrahydropyrido[3,4-d]pyrimidine, 1H-pyrazolo[3,4-d]pyrimidine, thieno[2,3-d]pyrimidine and thiazolo[5,4-d]pyrimidine, each optionally substituted with 1, 2, 3, 4 or 5 R^{f} substituents.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R¹ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R¹ is selected from hydrogen, halogen, CN, C₁₋₆ alkyl, C₁₋₆ haloalkyl, hydroxyl, C₁₋₆ alkoxy, NH₂, NHC₁₋₆ alkyl, and N(C₁₋₆ alkyl)₂.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R¹ is selected from hydrogen, halogen, CN, CF₃ and methyl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R¹ is hydrogen.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R³ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R³ is selected from hydrogen, halogen, CN, C₁₋₆ alkyl, C₁₋₆ haloalkyl, hydroxyl, C₁₋₆ alkoxy, NH₂, NHC₁₋₆ alkyl, and N(C₁₋₆ alkyl)₂.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R³ is selected from hydrogen, halogen, CN, CF₃ and methyl.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R³ is hydrogen.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R¹ and R³ are each hydrogen.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R⁴ is selected from hydrogen, C₁₋₆ alkyl, C₁₋₆ haloalkyl, C(O)R^{a}, C(O)OR^{a}, C(O)NR^{b}R^{c} and SO₂R^{a}.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n), XII(o) or XIII, R⁴ is hydrogen.

In some embodiments of compounds of Formula I, II, III, IV(a), IV(b), V(a), V(b), VI, VII(a), VII(b), VIII(a), VIII(b), IX, X(a), X(b), XI(a), XI(b), XII(a), XII(b), XII(c), XII(d), XII(e), XII(f), XII(g), XII(h), XII(i), XII(j), XII(k), XII(l), XII(m), XII(n) or XII(o), R¹, R³ and R⁴ are each hydrogen.

In some embodiments of compounds of Formula I, III, IV(b), V(b), VI, VII(b), VIII(b), IX, X(b), XI(b), XII(a), XII(c), XII(e), XII(f), XII(h), XII(j), XII(k), XII(m), XII(o) or XIII, R⁸ and R⁹, at each occurrence, are each independently selected from hydrogen, halogen and C₁₋₆ alkyl.

In some embodiments of compounds of Formula I, III, IV(b), V(b), VI, VII(b), VIII(b), IX, X(b), XI(b), XII(a), XII(c), XII(e), XII(f), XII(h), XII(j), XII(k), XII(m), XII(o) or XIII, R⁸ and R⁹, at each occurrence, are each hydrogen.

In some embodiments, a compound of Formula I is 1-(2-((3-fluoro-1-(3-fluoropyridin-2-yl)cyclobutyl)methylamino)pyrimidin-5-yl)-1H-pyrrole-3-carboxamide or a pharmaceutically acceptable salt thereof. In some embodiments, a compound of Formula I is
1-(2-(((trans)-3-fluoro-1-(3-fluoropyridin-2-yl)cyclobutyl)methylamino)pyrimidin-5-yl)-1H-pyrrole-3-carboxamide (Compound C) or a pharmaceutically acceptable salt thereof. In some embodiments, a compound of Formula I is
3-(2-((-3-fluoro-1-(3-fluoropyridin-2-yl)cyclobutyl)methylamino)pyrimidin-5-yl)benzamide or a pharmaceutically acceptable salt thereof. In some embodiments, a compound of Formula I is
3-(2-(((trans)-3-fluoro-1-(3-fluoropyridin-2-yl)cyclobutyl)methylamino)pyrimidin-5-yl)benzamide or a pharmaceutically acceptable salt thereof.

In some embodiments, the skeletal muscle troponin activate is a chemical entity chosen from compounds of Formula A and compounds of Formula B:

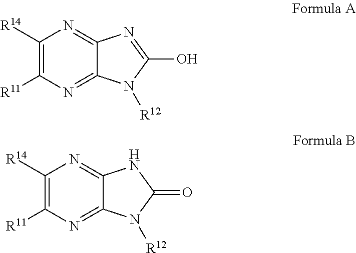

and pharmaceutically acceptable salts thereof, wherein

R¹¹ and R¹⁴ are independently selected from hydrogen, halo, hydroxy, optionally substituted acyl, optionally substituted alkyl, optionally substituted amino, optionally substituted alkenyl, optionally substituted alkynyl, optionally substituted cycloalkyl, optionally substituted heteroaryl, optionally substituted alkoxy, optionally substituted aminocarbonyl, sulfonyl, sulfanyl, sulfinyl, carboxy, optionally substituted alkoxycarbonyl, and cyano; and in the alternative, R¹⁴ and R¹¹, taken together with any intervening atoms, form a fused ring system selected from optionally substituted fused aryl, optionally substituted fused heteroaryl, optionally substituted fused cycloalkyl, and optionally substituted fused heterocycloalkyl; and

R¹² is selected from optionally substituted alkyl, optionally substituted cycloalkyl, optionally substituted aryl, optionally substituted heteroaryl, and optionally substituted heterocycloalkyl; provided that

R¹¹ is not hex-1-enyl; and further provided that the compound of Formula XIV or the compound of Formula XV is not

(S)-6-bromo-1-(1-phenylethyl)-1H-imidazo[4,5-b]pyrazin-2(3H)-one;

1,5,6-trimethyl-1H-imidazo[4,5-b]pyrazin-2(3H)-one;

1-methyl-1H-imidazo[4,5-b]pyrazin-2(3H)-one;

6-bromo-1-(3-nitrobenzyl)-1H-imidazo[4,5-b]pyrazin-2(3H)-one;

5-(hydroxymethyl)-1,6-dimethyl-1H-imidazo[4,5-b]pyrazin-2(3H)-one; or

1-(piperidin-4-yl)-1H-imidazo[4,5-b]pyrazin-2(3H)-one.

In some embodiments, R¹² is selected from optionally substituted lower alkyl, optionally substituted cycloalkyl, optionally substituted alkoxy, and optionally substituted heterocycloalkyl.

In some embodiments, R¹² is selected from heterocycloalkyl, cycloalkyl, lower alkyl, and lower alkyl substituted with optionally substituted phenyl, hydroxy, optionally substituted alkoxy, optionally substituted amino and optionally substituted heterocycloalkyl.

In some embodiments, R¹² is selected from 1-(R)-phenylethyl, 1-(S)-phenylethyl, benzyl, 3-pentyl, 4-heptyl, 4-methyl-1-morpholinopentan-2-yl isobutyl, cyclohexyl, cyclopropyl, sec-butyl, tert-butyl, isopropyl, 1-hydroxybutan-2-yl, tetrahydro-2H-pyran-4-yl, 1-methoxybutan-2-yl, 1-aminobutan-2-yl, and 1-morpholinobutan-2-yl.

In some embodiments, R¹¹ is selected from hydrogen, halo, acyl, optionally substituted lower alkyl, optionally substituted amino, optionally substituted pyrazolyl, optionally substituted alkenyl, optionally substituted alkynyl, optionally substituted lower alkoxy, and —S-(optionally substituted lower alkyl).

In some embodiments, R¹¹ is selected from hydrogen, halo, acyl, optionally substituted lower alkyl, dialkylamino, amino substituted with an alkyl group and with a group chosen from acyl, aminocarbonyl, alkoxycarbonyl, and sulfonyl; optionally substituted pyrazolyl, optionally substituted alkenyl, optionally substituted alkynyl, optionally substituted lower alkoxy, and —S-(optionally substituted lower alkyl).

In some embodiments, R¹¹ is selected from hydrogen, halo, acyl, alkenyl, alkynyl, lower alkoxy, optionally substituted amino, pyrazolyl substituted with lower alkyl, —S-(optionally substituted lower alkyl), lower alkyl, and lower alkyl substituted with halo.

In some embodiments, R¹¹ is selected from hydrogen, halo, acyl, alkenyl, alkynyl, lower alkoxy, dialkylamino, amino substituted with an alkyl group and with a group chosen from acyl, aminocarbonyl, alkoxycarbonyl, and sulfonyl, pyrazolyl substituted with lower alkyl, —S-(optionally substituted lower alkyl), lower alkyl, and lower alkyl substituted with halo.

In some embodiments, R¹¹ is selected from hydrogen, bromo, chloro, fluoro, methyl, ethyl, propyl, hexenyl, butenyl, propenyl, vinyl, ethynyl, methoxy, ethoxy, methylsulfanyl, dimethylamino, and methyl substituted with up to three fluoro groups.

In some embodiments, R¹¹ is selected from hydrogen, bromo, chloro, fluoro, methyl, ethyl, n-propyl, isopropyl, dimethylamino, isobuten-1-yl, (Z)-propen-1-yl, (E)-propen-1-yl, propen-2-yl, vinyl, ethynyl, methoxy, ethoxy, methylsulfanyl, and trifluoromethyl.

In some embodiments, R¹⁴ is selected from hydrogen, halo, acyl, optionally substituted alkyl, alkenyl, optionally substituted cycloalkyl, optionally substituted aminocarbonyl, sulfanyl, optionally substituted amino, and optionally substituted alkoxycarbonyl.

In some embodiments, R¹⁴ is selected from hydrogen, halo, acyl, optionally substituted lower alkyl, lower alkenyl, optionally substituted cycloalkyl, optionally substituted aminocarbonyl, sulfanyl, optionally substituted amino, and optionally substituted lower alkoxycarbonyl.

In some embodiments, R¹⁴ is selected from hydrogen, halo, acyl, lower alkyl, lower alkenyl, cycloalkyl, optionally substituted aminocarbonyl, sulfanyl, and lower alkoxycarbonyl.

In some embodiments, R¹⁴ is selected from hydrogen, bromo, chloro, fluoro, acetyl, methyl, ethyl, vinyl, cyclohexen-1-yl, methylcarbamoyl, dimethylcarbamoyl, methylsulfanyl, and methoxycarbonyl.

In some embodiments, R¹⁴ is hydrogen.

In some embodiments, R¹⁴ and R¹¹, taken together with any intervening atoms, form a fused ring system selected from optionally substituted fused aryl, optionally substituted fused cycloalkyl, and optionally substituted fused heterocycloalkyl.

In some embodiments, R¹⁴ and R¹¹ are taken together to form an optionally substituted benzo group.

In some embodiments, R¹⁴ and R¹¹ are taken together to form a benzo group.

In some embodiments, the skeletal muscle troponin activator is a chemical entity selected from compounds of Formula A and compounds of Formula B:

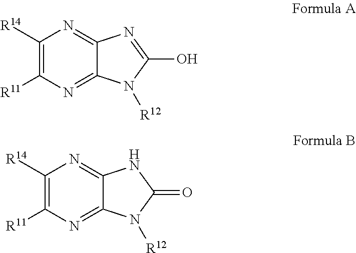

or a pharmaceutically acceptable salt thereof, wherein:

R¹¹ is alkenyl or alkynyl;

R¹⁴ is hydrogen; and

R¹² is selected from 3-pentyl, 4-heptyl, 4-methyl-1-morpholinopentan-2-yl isobutyl, cyclohexyl, cyclopropyl, sec-butyl, tert-butyl, isopropyl, 1-hydroxybutan-2yl, tetrahydro-2H-pyran-4-yl, 1-methoxybutan-2-yl, 1-aminobutan-2-yl, and 1-morpholinobutan-2-yl;

provided that R¹¹ is not hex-1-enyl.
In some embodiments, the compound of Formula A is chosen from
   1-((1R)-1-methyl-2-morpholin-4-ylethyl)-6-bromoimidazo[4,5-b]pyrazin-2-ol;
   1-(ethylpropyl)-6-ethynylimidazo[4,5-b]pyrazin-2-ol;
   1-(ethylpropyl)-6-methoxyimidazo[4,5-b]pyrazin-2-ol;
   1-(1,1-dimethyl-2-morpholin-4-ylethyl)-6-bromoimidazo[4,5-b]pyrazin-2-ol;
   6-(1H-1,2,3-triazol-4-yl)-1-(ethylpropyl)imidazo[4,5-b ]pyrazin-2-ol;
   1-(ethylpropyl)-6-(trifluoromethyl)imidazo[4,5-b ]pyrazin-2-ol;
   1-[(1R)-1-(morpholin-4-ylmethyl)propyl]-6-ethynylimidazo[4,5-b]pyrazin-2-ol;
   1-(ethylpropyl)-6-{2-[1-(ethylpropyl)-2-hydroxyimidazo[4,5-e]pyrazin-6-yl]ethynyl}imidazo[4,5-b]pyrazin-2-ol;
   6-(dimethylamino)-1-(ethylpropyl)imidazo[4,5-b]pyrazin-2-ol;
   6-ethyl-1-(ethylpropyl)imidazo[4,5-b]pyrazin-2-ol;
   (E)-1-(pentan-3-yl)-6-(prop-1-enyl)-1H-imidazo[4,5-b]pyrazin-2-ol;
   (E)-1-cyclohexyl-6-(prop-1-enyl)-1H-imidazo[4,5-b]pyrazin-2-ol;
   (E)-1-cyclopropyl-6-(prop-1-enyl)-1H-imidazo[4,5-b]pyrazin-2-ol;
   (E)-1-isopropyl-6-(prop-1-enyl)-1H-imidazo[4,5-b]pyrazin-2-ol;
   (E)-6-(prop-1-enyl)-1-(tetrahydro-2H-pyran-4-yl)-1H-imidazo[4,5-b]pyrazin-2-ol;
   (R)-6-(methylthio)-1-(1-phenylethyl)-1H-imidazo[4,5-b]pyrazin-2-ol;
   (R)-6-bromo-1-(1-hydroxybutan-2-yl)-1H-imidazo[4,5-b]pyrazin-2-ol;
   (R)-6-bromo-1-(1-morpholinobutan-2-yl)-1H-imidazo[4,5-b]pyrazin-2-ol;
   (R)-6-bromo-1-(1-morpholinopropan-2-yl)-1H-imidazo[4,5-b]pyrazin-2-ol;
   (R)-6-bromo-1-(1-phenylethyl)-1H-imidazo[4,5-b]pyrazin-2-ol;
   (R)-6-bromo-1-sec-butyl-1H-imidazo[4,5-b]pyrazin-2-ol;
   (S)-(2-hydroxy-1-(1-phenylethyl)-1H-imidazo[4,5-b]pyrazin-6-yl)(4-methylpiperazin-1-yl)methanone;
   (S)-(2-hydroxy-1-(1-phenylethyl)-1H-imidazo[4,5-b]pyrazin-6-yl)(morpholino)methanone;
   (S)-(2-hydroxy-1-(1-phenylethyl)-1H-imidazo[4,5-b]pyrazin-6-yl)(piperidin-1-yl)methanone;
   (S)-1-(1-phenylethyl)-1H-imidazo[4,5-b]pyrazin-2-ol;
   (S)-1-(1-phenylethyl)-1H-imidazo[4,5-b]quinoxalin-2-ol;
   (S)-1-(1-phenylethyl)-6-(piperidin-1-ylmethyl)-1H-imidazo[4,5-b]pyrazin-2-ol;
   (S)-1-(1-phenylethyl)-6-propyl-1H-imidazo[4,5-b]pyrazin-2-ol;
   (S)-1-(1-phenylethyl)-6-vinyl-1H-imidazo[4,5-b]pyrazin-2-ol;
   (S)-1-(2-hydroxy-1-(1-phenylethyl)-1H-imidazo[4,5-b]pyrazin-6-yl)ethanone;
   (S)-2-hydroxy-1-(1-phenylethyl)-1H-imidazo[4,5-b]pyrazine-6-carbonitrile;
   (S)-2-hydroxy-1-(1-phenylethyl)-1H-imidazo[4,5-b]pyrazine-6-carboxamide;
   (S)-2-hydroxy-1-(1-phenylethyl)-1H-imidazo[4,5-b]pyrazine-6-carboxylic acid;
   (S)-2-hydroxy-N,N-dimethyl-1-(1-phenylethyl)-1H-imidazo[4,5-b]pyrazine-6-carboxamide;
   (S)-2-hydroxy-N-methyl-1-(1-phenylethyl)-1H-imidazo[4,5-b]pyrazine-6-carboxamide;
   (S)-6-((4-methylpiperazin-1-yl)methyl)-1-(1-phenylethyl)-1H-imidazo[4,5-b]pyrazin-2-ol;
   (S)-6-((dimethylamino)methyl)-1-(1-phenylethyl)-1H-imidazo[4,5-b]pyrazin-2-ol;
   (S)-6-(2-hydroxypropan-2-yl)-1-(1-phenylethyl)-1H-imidazo[4,5-b]pyrazin-2-ol;
   (S)-6-(2-methylprop-1-enyl)-1-(1-phenylethyl)-1H-imidazo[4,5-b]pyrazin-2-ol;
   (S)-6-(methylsulfonyl)-1-(1-phenylethyl)-1H-imidazo[4,5-b]pyrazin-2-ol;
   (S)-6-(methylthio)-1-(1-phenylethyl)-1H-imidazo[4,5-b]pyrazin-2-ol;
   (S)-6-(morpholinomethyl)-1-(1-phenylethyl)-1H-imidazo[4,5-b]pyrazin-2-ol;
   (S)-6-bromo-1-(1-hydroxybutan-2-yl)-1H-imidazo[4,5-b]pyrazin-2-ol;
   (S)-6-bromo-1-(1-morpholinobutan-2-yl)-1H-imidazo[4,5-b]pyrazin-2-ol;
   (S)-6-bromo-1-(1-morpholinopropan-2-yl)-1H-imidazo[4,5-b]pyrazin-2-ol;
   (S)-6-bromo-1-(1-phenylethyl)-1H-imidazo[4,5-b]pyrazin-2-ol;
   (S)-6-bromo-1-sec-butyl-1H-imidazo[4,5-b]pyrazin-2-ol;
   (S)-6-cyclohexenyl-1-(1-phenylethyl)-1H-imidazo[4,5-b]pyrazin-2-ol;
   (S)-6-cyclohexyl-1-(1-phenylethyl)-1H-imidazo[4,5-b]pyrazin-2-ol;
   (S)-6-ethoxy-1-(1-phenylethyl)-1H-imidazo[4,5-b]pyrazin-2-ol;
   (S)-6-ethyl-1-(1-phenylethyl)-1H-imidazo[4,5-b]pyrazin-2-ol;
   (S)-6-hexyl-1-(1-phenylethyl)-1H-imidazo[4,5-b]pyrazin-2-ol;
   (S)-6-isobutyl-1-(1-phenylethyl)-1H-imidazo[4,5-b]pyrazin-2-ol;
   (S)-6-methoxy-1-(1-phenylethyl)-1H-imidazo[4,5-b]pyrazin-2-ol;
   (S)-methyl 2-hydroxy-1-(1-phenylethyl)-1H-imidazo[4,5-b]pyrazine-6-carboxylate;
   (S)-N,N-diethyl-2-hydroxy-1-(1-phenylethyl)-1H-imidazo[4,5-b]pyrazine-6-carboxamide;
   (S)-N-benzyl-2-hydroxy-1-(1-phenylethyl)-1H-imidazo[4,5-b]pyrazine-6-carboxamide;
   (S,E)-1-(1-phenylethyl)-6-(prop-1-enyl)-1H-imidazo[4,5-b]pyrazin-2-ol;
   (S,Z)-1-(1-phenylethyl)-6-(prop-1-enyl)-1H-imidazo[4,5-b]pyrazin-2-ol;
   (S,Z)-6-(hex-2-enyl)-1-(1-phenylethyl)-1H-imidazo[4,5-b]pyrazin-2-ol;
   (Z)-1-(pentan-3-yl)-6-(prop-1-enyl)-1H-imidazo[4,5-b]pyrazin-2-ol;
   (Z)-1-cyclohexyl-6-(prop-1-enyl)-1H-imidazo[4,5-b]pyrazin-2-ol;
   (Z)-1-cyclopropyl-6-(prop-1-enyl)-1H-imidazo[4,5-b]pyrazin-2-ol;
   (Z)-1-isopropyl-6-(prop-1-enyl)-1H-imidazo[4,5-b]pyrazin-2-ol;
   (Z)-6-(prop-1-enyl)-1-(tetrahydro-2H-pyran-4-yl)-1H-imidazo[4,5-b]pyrazin-2-ol;
   1-(1-aminobutan-2-yl)-6-bromo-1H-imidazo[4,5-b]pyrazin-2-ol;
   1-(1-morpholinobutan-2-yl)-1H-imidazo[4,5-b]pyrazin-2-ol;
   1-(2-hydroxy-1-(pentan-3-yl)-1H-imidazo[4,5-b]pyrazin-5-yl)ethanone;
   1-(pentan-3-yl)-1H-imidazo[4,5-b]pyrazin-2-ol;
   1-(pentan-3-yl)-1H-imidazo[4,5-b]pyrazine-2,6-diol;
   1-(pentan-3-yl)-1H-imidazo[4,5-b]quinoxalin-2-ol;
   1-(pentan-3-yl)-5-vinyl-1H-imidazo[4,5-b]pyrazin-2-ol;
   1-(pentan-3-yl)-6-(prop-1-ynyl)-1H-imidazo[4,5-b]pyrazin-2-ol;
   1-(pentan-3-yl)-6-(trifluoromethyl)-1H-imidazo[4,5-b]pyrazin-2-ol;
   1-benzyl-6-(methylthio)-1H-imidazo[4,5-b]pyrazin-2-ol;
   1-benzyl-6-bromo-1H-imidazo[4,5-b]pyrazin-2-ol;
   1-cyclohexyl-6-(methylthio)-1H-imidazo[4,5-b]pyrazin-2-ol;
   1-cyclopropyl-6-(methylthio)-1H-imidazo[4,5-b]pyrazin-2-ol;
   1-isopropyl-6-(methylthio)-1H-imidazo[4,5-b]pyrazin-2-ol;
   2-(6-bromo-2-hydroxy-1H-imidazo[4,5-b]pyrazin-1-yl)-1-morpholinobutan-1-one;
   2-(6-bromo-2-hydroxy-1H-imidazo[4,5-b]pyrazin-1-yl)butanoic acid;
   2-(6-bromo-2-hydroxy-1H-imidazo[4,5-b]pyrazin-1-yl)propane-1,3-diol;
   2-hydroxy-1-(pentan-3-yl)-1H-imidazo[4,5-b]pyrazine-5-carboxylic acid;
   2-hydroxy-1-(pentan-3-yl)-1H-imidazo[4,5-b]pyrazine-6-carbonitrile;
   2-hydroxy-N,N-dimethyl-1-(pentan-3-yl)-1H-imidazo[4,5-b]pyrazine-5-carboxamide;
   2-hydroxy-N-methyl-1-(pentan-3-yl)-1H-imidazo[4,5-b]pyrazine-5-carboxamide;
   5-(methylthio)-1-(pentan-3-yl)-1H-imidazo[4,5-b]pyrazin-2-ol;
   5-bromo-1-(pentan-3-yl)-1H-imidazo[4,5-b]pyrazin-2-ol;
   5-ethyl-1-(pentan-3-yl)-1H-imidazo[4,5-b]pyrazin-2-ol;
   6-(methylsulfinyl)-1-((S)-1-phenylethyl)-1H-imidazo[4,5-b]pyrazin-2-ol;
   6-(methylthio)-1-(pentan-3-yl)-1H-imidazo[4,5-b]pyrazin-2-ol;
   6-(methylthio)-1-(tetrahydro-2H-pyran-4-yl)-1H-imidazo[4,5-b]pyrazin-2-ol;
   6-bromo-1-(1-(4-(methylsulfonyl)piperazin-1-yl)butan-2-yl)-1H-imidazo[4,5-b]pyrazin-2-ol;
   6-bromo-1-(1-(4-methylpiperazin-1-yl)butan-2-yl)-1H-imidazo[4,5-b]pyrazin-2-ol;
   6-bromo-1-(1-(dimethylamino)butan-2-yl)-1H-imidazo[4,5-b]pyrazin-2-ol;
   6-bromo-1-(1-(methylamino)butan-2-yl)-1H-imidazo[4,5-b]pyrazin-2-ol;
   6-bromo-1-(1-methoxybutan-2-yl)-1H-imidazo[4,5-b]pyrazin-2-ol;
   6-bromo-1-(2-methyl-1-morpholinopropan-2-yl)-1H-imidazo[4,5-b]pyrazin-2-ol;
   6-bromo-1-(2-morpholinoethyl)-1H-imidazo[4,5-b]pyrazin-2-ol;
   6-bromo-1-(pentan-3-yl)-1H-imidazo[4,5-b]pyrazin-2-ol;
   6-bromo-1-(tetrahydro-2H-pyran-4-yl)-1H-imidazo[4,5-b]pyrazin-2-ol;
   6-bromo-1-cyclohexyl-1H-imidazo[4,5-b]pyrazin-2-ol;
   6-bromo-1-cyclopropyl-1H-imidazo[4,5-b]pyrazin-2-ol;
   6-bromo-1-isopropyl-1H-imidazo[4,5-b]pyrazin-2-ol;
   6-bromo-1-tert-butyl-1H-imidazo[4,5-b]pyrazin-2-ol;
   6-cyclopropyl-1-(pentan-3-yl)-1H-imidazo[4,5-b]pyrazin-2-ol;
   6-ethynyl-1-(pentan-3-yl)-1H-imidazo[4,5-b]pyrazin-2-ol;
   6-methoxy-1-(pentan-3-yl)-1H-imidazo[4,5-b]pyrazin-2-ol;
   6-methyl-1-(pentan-3-yl)-1H-imidazo[4,5-b]pyrazin-2-ol;
   methyl 2-hydroxy-1-(pentan-3-yl)-1H-imidazo[4,5-b]pyrazine-5-carboxylate;
   methyl 4-(2-(6-bromo-2-hydroxy-1H-imidazo[4,5-b]pyrazin-1-yl)butyl)piperazine-1-carboxylate;
   1-(ethylpropyl)-6-(1-methylpyrazol-4-yl)imidazo[4,5-13]pyrazin-2-ol;
   6-bromo-1-(propylbutyl)imidazo[4,5-b]pyrazin-2-ol;
   1-[(1R)-3-methyl-1-(morpholin-4-ylmethyl)butyl]-6-bromoimidazo[4,5-b]pyrazin-2-ol;
   1-(ethylpropyl)-6-vinylimidazo[4,5-b]pyrazin-2-ol;
   1-(ethylpropyl)-6-(1-methylvinyl)imidazo[4,5-b]pyrazin-2-ol;
   1-(ethylpropyl)-6-(methylethyl)imidazo[4,5-b]pyrazin-2-ol;
   6-chloro-1-(ethylpropyl)imidazo[4,5-b]pyrazin-2-ol; and
   6-(dimethylamino)-1-(ethylpropyl)imidazo[4,5-b]pyrazin-2-ol, or a pharmaceutically acceptable salt thereof.

In some embodiments, the compound of Formula B is chosen from the following tautomers of compounds of Formula A:
(R)-6-bromo-1-(1-morpholinopropan-2-yl)-1H-imidazo[4,5-b]pyrazin-2(3H)-one;
6-ethynyl-1-(pentan-3-yl)-1H-imidazo[4,5-b]pyrazin-2(3H)-one;
6-methoxy-1-(pentan-3-yl)-1H-imidazo[4,5-b]pyrazin-2(3H)-one;
6-bromo-1-(2-methyl-1-morpholinopropan-2-yl)-1H-imidazo[4,5-b]pyrazin-2(3H)-one;
1-(pentan-3-yl)-6-(1H-1,2,3-triazol-4-yl)-1H-imidazo[4,5-b]pyrazin-2(3H)-one;
1-(pentan-3-yl)-6-(trifluoromethyl)-1H-imidazo[4,5-b]pyrazin-2(3H)-one;
(R)-6-ethynyl-1-(1-morpholinobutan-2-yl)-1H-imidazo[4,5-b]pyrazin-2(3H)-one;
6-((2-hydroxy-1-(pentan-3-yl)-1H-imidazo[4,5-b]pyrazin-6-yl)ethynyl)-1-(pentan-3-yl)-1H-imidazo[4,5-b]pyrazin-2(3H)-one;
6-(dimethylamino)-1-(pentan-3-yl)-1H-imidazo[4,5-b]pyrazin-2(3H)-one;
6-ethyl-1-(pentan-3-yl)-1H-imidazo[4,5-b]pyrazin-2(3H)-one;
(E)-1-(pentan-3-yl)-6-(prop-1-enyl)-1H-imidazo[4,5-b]pyrazin-2(3H)-one;
(E)-1-cyclohexyl-6-(prop-1-enyl)-1H-imidazo[4,5-b]pyrazin-2(3H)-one;
(E)-1-cyclopropyl-6-(prop-1-enyl)-1H-imidazo[4,5-b]pyrazin-2(3H)-one;
(E)-1-isopropyl-6-(prop-1-enyl)-1H-imidazo[4,5-b]pyrazin-2(3H)-one;
(E)-6-(prop-1-enyl)-1-(tetrahydro-2H-pyran-4-yl)-1H-imidazo[4,5-b]pyrazin-2(3H)-one;
(R)-6-(methylthio)-1-(1-phenylethyl)-1H-imidazo[4,5-b]pyrazin-2(3H)-one;
(R)-6-bromo-1-(1-hydroxybutan-2-yl)-1H-imidazo[4,5-b]pyrazin-2(3H)-one;
(R)-6-bromo-1-(1-morpholinobutan-2-yl)-1H-imidazo[4,5-b]pyrazin-2(3H)-one;
(R)-6-bromo-1-(1-morpholinopropan-2-yl)-1H-imidazo[4,5-b]pyrazin-2(3H)-one;
(R)-6-bromo-1-(1-phenylethyl)-1H-imidazo[4,5-b]pyrazin-2(3H)-one;
(R)-6-bromo-1-sec-butyl-1H-imidazo[4,5-b]pyrazin-2(3H)-one;
(S)-1-(1-phenylethyl)-1H-imidazo[4,5-b]pyrazin-2(3H)-one;
(S)-1-(1-phenylethyl)-1H-imidazo[4,5-b]quinoxalin-2(3H)-one;
(S)-1-(1-phenylethyl)-6-(piperidin-1-ylmethyl)-1H-imidazo[4,5-b]pyrazin-2(3H)-one;
(S)-1-(1-phenylethyl)-6-(piperidine-1-carbonyl)-1H-imidazo[4,5-b]pyrazin-2(3H)-one;
(S)-1-(1-phenylethyl)-6-propyl-1H-imidazo[4,5-b]pyrazin-2(3H)-one;
(S)-1-(1-phenylethyl)-6-vinyl-1H-imidazo[4,5-b]pyrazin-2(3H)-one;
(S)-2-oxo-3-(1-phenylethyl)-2,3-dihydro-1H-imidazo[4,5-b]pyrazine-5-carbonitrile;
(S)-2-oxo-3-(1-phenylethyl)-2,3-dihydro-1H-imidazo[4,5-b]pyrazine-5-carboxamide;
(S)-2-oxo-3-(1-phenylethyl)-2,3-dihydro-1H-imidazo[4,5-b]pyrazine-5-carboxylic acid;
(S)-6-((4-methylpiperazin-1-yl)methyl)-1-(1-phenylethyl)-1H-imidazo[4,5-b]pyrazin-2(3H)-one;
(S)-6-((dimethylamino)methyl)-1-(1-phenylethyl)-1H-imidazo[4,5-b]pyrazin-2(3H)-one;
(S)-6-(2-hydroxypropan-2-yl)-1-(1-phenylethyl)-1H-imidazo[4,5-b]pyrazin-2(3H)-one;
(S)-6-(2-methylprop-1-enyl)-1-(1-phenylethyl)-1H-imidazo[4,5-b]pyrazin-2(3H)-one;
(S)-6-(4-methylpiperazine-1-carbonyl)-1-(1-phenylethyl)-1H-imidazo[4,5-b]pyrazin-2(3H)-one;
(S)-6-(methylsulfonyl)-1-(1-phenylethyl)-1H-imidazo[4,5-b]pyrazin-2(3H)-one;
(S)-6-(methylthio)-1-(1-phenylethyl)-1H-imidazo[4,5-b]pyrazin-2(3H)-one;
(S)-6-(morpholine-4-carbonyl)-1-(1-phenylethyl)-1H-imidazo[4,5-b]pyrazin-2(3H)-one;
(S)-6-(morpholinomethyl)-1-(1-phenylethyl)-1H-imidazo[4,5-b]pyrazin-2(3H)-one;
(S)-6-acetyl-1-(1-phenylethyl)-1H-imidazo[4,5-b]pyrazin-2(3H)-one;
(S)-6-bromo-1-(1-hydroxybutan-2-yl)-1H-imidazo[4,5-b]pyrazin-2(3H)-one;
(S)-6-bromo-1-(1-morpholinobutan-2-yl)-1H-imidazo[4,5-b]pyrazin-2(3H)-one;
(S)-6-bromo-1-(1-morpholinopropan-2-yl)-1H-imidazo[4,5-b]pyrazin-2(3H)-one;
(S)-6-bromo-1-(1-phenylethyl)-1H-imidazo[4,5-b]pyrazin-2(3H)-one;
(S)-6-bromo-1-sec-butyl-1H-imidazo[4,5-b]pyrazin-2(3H)-one;
(S)-6-cyclohexenyl-1-(1-phenylethyl)-1H-imidazo[4,5-b]pyrazin-2(3H)-one;
(S)-6-cyclohexyl-1-(1-phenylethyl)-1H-imidazo[4,5-b]pyrazin-2(3H)-one;
(S)-6-ethoxy-1-(1-phenylethyl)-1H-imidazo[4,5-b]pyrazin-2(3H)-one;
(S)-6-ethyl-1-(1-phenylethyl)-1H-imidazo[4,5-b]pyrazin-2(3H)-one;
(S)-6-hexyl-1-(1-phenylethyl)-1H-imidazo[4,5-b]pyrazin-2(3H)-one;
(S)-6-isobutyl-1-(1-phenylethyl)-1H-imidazo[4,5-b]pyrazin-2(3H)-one;
(S)-6-methoxy-1-(1-phenylethyl)-1H-imidazo[4,5-b]pyrazin-2(3H)-one;
(S)-methyl 2-oxo-3-(1-phenylethyl)-2,3-dihydro-1H-imidazo[4,5-b]pyrazine-5-carboxylate;
(S)-N,N-diethyl-2-oxo-3-(1-phenylethyl)-2,3-dihydro-1H-imidazo[4,5-b]pyrazine-5-carboxamide;

(S)-N,N-dimethyl-2-oxo-3-(1-phenylethyl)-2,3-dihydro-1H-imidazo[4,5-b]pyrazine-5-carboxamide;
(S)-N-benzyl-2-oxo-3-(1-phenylethyl)-2,3-dihydro-1H-imidazo[4,5-b]pyrazine-5-carboxamide;
(S)-N-methyl-2-oxo-3-(1-phenylethyl)-2,3-dihydro-1H-imidazo[4,5-b]pyrazine-5-carboxamide;
(S,E)-1-(1-phenylethyl)-6-(prop-1-enyl)-1H-imidazo[4,5-b]pyrazin-2(3H)-one;
(S,Z)-1-(1-phenylethyl)-6-(prop-1-enyl)-1H-imidazo[4,5-b]pyrazin-2(3H)-one;
(S,Z)-6-(hex-2-enyl)-1-(1-phenylethyl)-1H-imidazo[4,5-b]pyrazin-2(3H)-one;
(Z)-1-(pentan-3-yl)-6-(prop-1-enyl)-1H-imidazo[4,5-b]pyrazin-2(3H)-one;
(Z)-1-cyclohexyl-6-(prop-1-enyl)-1H-imidazo[4,5-b]pyrazin-2(3H)-one;
(Z)-1-cyclopropyl-6-(prop-1-enyl)-1H-imidazo[4,5-b]pyrazin-2(3H)-one;
(Z)-1-isopropyl-6-(prop-1-enyl)-1H-imidazo[4,5-b]pyrazin-2(3H)-one;
(Z)-6-(prop-1-enyl)-1-(tetrahydro-2H-pyran-4-yl)-1H-imidazo[4,5-b]pyrazin-2(3H)-one;
1-(1-aminobutan-2-yl)-6-bromo-1H-imidazo[4,5-b]pyrazin-2(3H)-one;
1-(1-morpholinobutan-2-yl)-1H-imidazo[4,5-b]pyrazin-2(3H)-one;
1-(pentan-3-yl)-1H-imidazo[4,5-b]pyrazin-2(3H)-one;
1-(pentan-3-yl)-1H-imidazo[4,5-b]quinoxalin-2(3H)-one;
1-(pentan-3-yl)-5-vinyl-1H-imidazo[4,5-b]pyrazin-2(3H)-one;
1-(pentan-3-yl)-6-(prop-1-ynyl)-1H-imidazo[4,5-b]pyrazin-2(3H)-one;
1-(pentan-3-yl)-6-(trifluoromethyl)-1H-imidazo[4,5-b]pyrazin-2(3H)-one;
1-benzyl-6-(methylthio)-1H-imidazo[4,5-b]pyrazin-2(3H)-one;
1-benzyl-6-bromo-1H-imidazo[4,5-b]pyrazin-2(3H)-one;
1-cyclohexyl-6-(methylthio)-1H-imidazo[4,5-b]pyrazin-2(3H)-one;
1-cyclopropyl-6-(methylthio)-1H-imidazo[4,5-b]pyrazin-2(3H)-one;
1-isopropyl-6-(methylthio)-1H-imidazo[4,5-b]pyrazin-2(3H)-one;
2-(6-bromo-2-oxo-2,3-dihydro-1H-imidazo[4,5-b]pyrazin-1-yl)butanoic acid;
2-oxo-1-(pentan-3-yl)-2,3-dihydro-1H-imidazo[4,5-b]pyrazine-5-carboxylic acid;
2-oxo-3-(pentan-3-yl)-2,3-dihydro-1H-imidazo[4,5-b]pyrazine-5-carbonitrile;
5-(methylthio)-1-(pentan-3-yl)-1H-imidazo[4,5-b]pyrazin-2(3H)-one;
5-acetyl-1-(pentan-3-yl)-1H-imidazo[4,5-b]pyrazin-2(3H)-one;
5-bromo-1-(pentan-3-yl)-1H-imidazo[4,5-b]pyrazin-2(3H)-one;
5-ethyl-1-(pentan-3-yl)-1H-imidazo[4,5-b]pyrazin-2(3H)-one;
6-(methylsulfinyl)-1-((S)-1-phenylethyl)-1H-imidazo[4,5-b]pyrazin-2(3H)-one;
6-(methylthio)-1-(pentan-3-yl)-1H-imidazo[4,5-b]pyrazin-2(3H)-one;
6-(methylthio)-1-(tetrahydro-2H-pyran-4-yl)-1H-imidazo[4,5-b]pyrazin-2(3H)-one;
6-bromo-1-(1-(4-(methylsulfonyl)piperazin-1-yl)butan-2-yl)-1H-imidazo[4,5-b]pyrazin-2(3H)-one;
6-bromo-1-(1-(4-methylpiperazin-1-yl)butan-2-yl)-1H-imidazo[4,5-b]pyrazin-2(3H)-one;
6-bromo-1-(1-(dimethylamino)butan-2-yl)-1H-imidazo[4,5-b]pyrazin-2(3H)-one;
6-bromo-1-(1-(methylamino)butan-2-yl)-1H-imidazo[4,5-b]pyrazin-2(3H)-one;
6-bromo-1-(1,3-dihydroxypropan-2-yl)-1H-imidazo[4,5-b]pyrazin-2(3H)-one;
6-bromo-1-(1-methoxybutan-2-yl)-1H-imidazo[4,5-b]pyrazin-2(3H)-one;
6-bromo-1-(1-morpholino-1-oxobutan-2-yl)-1H-imidazo[4,5-b]pyrazin-2(3H)-one;
6-bromo-1-(2-methyl-1-morpholinopropan-2-yl)-1H-imidazo[4,5-b]pyrazin-2(3H)-one;
6-bromo-1-(2-morpholinoethyl)-1H-imidazo[4,5-b]pyrazin-2(3H)-one;
6-bromo-1-(pentan-3-yl)-1H-imidazo[4,5-b]pyrazin-2(3H)-one;
6-bromo-1-(tetrahydro-2H-pyran-4-yl)-1H-imidazo[4,5-b]pyrazin-2(3H)-one;
6-bromo-1-cyclohexyl-1H-imidazo[4,5-b]pyrazin-2(3H)-one;
6-bromo-1-cyclopropyl-1H-imidazo[4,5-b]pyrazin-2(3H)-one;
6-bromo-1-isopropyl-1H-imidazo[4,5-b]pyrazin-2(3H)-one;
6-bromo-1-tert-butyl-1H-imidazo[4,5-b]pyrazin-2(3H)-one;
6-cyclopropyl-1-(pentan-3-yl)-1H-imidazo[4,5-b]pyrazin-2(3H)-one;
6-ethynyl-1-(pentan-3-yl)-1H-imidazo[4,5-b]pyrazin-2(3H)-one;
6-hydroxy-1-(pentan-3-yl)-1H-imidazo[4,5-b]pyrazin-2(3H)-one;
6-methoxy-1-(pentan-3-yl)-1H-imidazo[4,5-b]pyrazin-2(3H)-one;
6-methyl-1-(pentan-3-yl)-1H-imidazo[4,5-b]pyrazin-2(3H)-one;
methyl 2-oxo-1-(pentan-3-yl)-2,3-dihydro-1H-imidazo[4,5-b]pyrazine-5-carboxylate;
methyl 4-(2-(6-bromo-2-oxo-2,3-dihydro-1H-imidazo[4,5-b]pyrazin-1-yl)butyl)piperazine-1-carboxylate;
N,N-dimethyl-2-oxo-1-(pentan-3-yl)-2,3-dihydro-1H-imidazo[4,5-b]pyrazine-5-carboxamide;
N-methyl-2-oxo-1-(pentan-3-yl)-2,3-dihydro-1H-imidazo[4,5-b]pyrazine-5-carboxamide;
6-(1-methyl-1H-pyrazol-4-yl)-1-(pentan-3-yl)-1H-imidazo[4,5-b]pyrazin-2(3H)-one;
6-bromo-1-(heptan-4-yl)-1H-imidazo[4,5-b]pyrazin-2(3H)-one;
(R)-6-bromo-1-(4-methyl-1-morpholinopentan-2-yl)-1H-imidazo[4,5-b]pyrazin-2(3H)-one;
1-(pentan-3-yl)-6-vinyl-1H-imidazo[4,5-b]pyrazin-2(3H)-one;
1-(pentan-3-yl)-6-(prop-1-en-2-yl)-1H-imidazo[4,5-b]pyrazin-2(3H)-one;
6-isopropyl-1-(pentan-3-yl)-1H-imidazo[4,5-b]pyrazin-2(3H)-one;
6-chloro-1-(pentan-3-yl)-1H-imidazo[4,5-b]pyrazin-2(3H)-one; and
6-(dimethylamino)-1-(pentan-3-yl)-1H-imidazo[4,5-b]pyrazin-2(3H)-one, or a pharmaceutically acceptable salt thereof.

In some embodiments, the compound of Formula A is 6-bromo-1-(pentan-3-yl)-1H-imidazo[4,5-b]pyrazin-2-ol or a pharmaceutically acceptable salt thereof. In some embodiments, the compound of formula A is 6-ethynyl-1-(pentan-3-yl)-1H-imidazo[4,5-b]pyrazin-2-ol (Compound A) or a pharmaceutically acceptable salt thereof

The compounds of Formula A can be named and numbered (e.g., using NamExpert™ available from Cheminnovation or the automatic naming feature of ChemDraw Ultra version 10.0 from Cambridge Soft Corporation) as described below. For example, the compound:

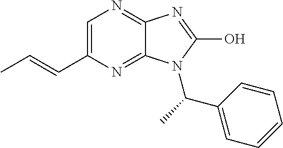 [SMILES: C/C=C/c1cnc2nc(O)n([C@@H](C)c3ccccc3)c2n1]

i.e., the compound according to Formula A where R¹¹ is (E)-propen-1yl, R¹² is (S)-sec-phenethyl, and R¹⁴ is H, can be named (S,E)-1-(1-phenylethyl)-6-(prop-1-enyl)-1H-imidazo[4,5-b]pyrazin-2-ol.

Likewise the compound:

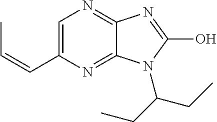 [SMILES: C/C=C\c1cnc2nc(O)n(C(CC)CC)c2n1]

i.e., the compound according to Formula A where R¹¹ is (Z)-propen-1-yl, R¹² is 3-pentyl, and R¹⁴ is H, can be named (Z)-1-(pentan-3-yl)-6-(prop-1-enyl)-1H-imidazo[4,5-b]pyrazin-2-ol.

Similarly, the compounds of Formula B can be named and numbered (e.g., using NamExpert™ available from Cheminnovation or the automatic naming feature of ChemDraw Ultra version 10.0 from Cambridge Soft Corporation) as described below. For example, the compound:

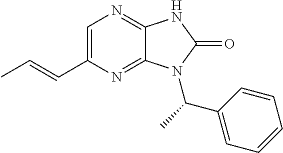 [SMILES: C/C=C/c1cnc2[nH]c(=O)n([C@@H](C)c3ccccc3)c2n1]

i.e., the compound according to Formula B where R¹¹ is (E)-propen-1-yl, R¹² is (S)-sec-phenethyl, and R¹⁴ is H, can be named (S,E)-1-(1-phenylethyl)-6-(prop-1-enyl)-1H-imidazo[4,5-b]pyrazin-2(3H)-one.

Likewise the compound:

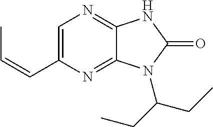 [SMILES: C/C=C\c1cnc2[nH]c(=O)n(C(CC)CC)c2n1]

i.e., the compound according to Formula B where R¹¹ is (Z)-propen-1-yl, R¹² is 3-pentyl, and R¹⁴ is H, can be named (Z)-1-(pentan-3-yl)-6-(prop-1-enyl)-1H-imidazo[4,5-b ]pyrazin-2(3H)-one.

Methods of preparing compounds of the present disclosure are readily available in the art. WO 2011/133888 provides synthesis methods for Formulas I-XIII. U.S. Pat. No. 7,956,056, for instance, discloses methods of preparing compounds of Formula A and Formula B.

It is also contemplated that skeletal muscle troponin activators suitable for methods of the present disclosure can be compounds disclosed in U.S. Pat. Nos. 8,227,603, 8,063,082, 7,989,469, 7,956,056, 7,851,484, and 7,598,248, and PCT Publication Nos. WO/2013/010015, WO/2011/0133922, WO/2011/0133920, WO/2011/133888, WO/2011/133882, WO/2009/099594, and WO/2008/016648. The contents of these patents and patent applications are incorporated into the present disclosure by references in their entirety.

The chemical entities described herein are useful for improving resistance to muscle fatigue in a subject in need thereof. The improvement in resistance to skeletal muscle fatigue in the subject may be determined by a bilateral heel-raise test, wherein the bilateral heel-raise test comprises performing heel raises at regular intervals; monitoring claudication symptoms; determining the value of one or more parameters selected from claudication onset, number of heel raises to claudication onset, work to claudication onset, time to maximal claudication fatigue, number of heel raises to maximal claudication fatigue, and work to maximal claudication fatigue; and wherein an increase in the one or more parameters indicates an improvement in resistance to fatigue in the subject. The bilateral heel raise test may be performed at any time after administration of a skeletal muscle troponin activator, e.g., about 1, 3, 6, 12, 24, or 48 or more hours after administration of the chemical entity.

In certain embodiments, the parameter is time to claudication onset. In certain embodiments, the parameter is number of heel raises to claudication onset, work to claudication onset, time to maximal claudication fatigue, number of heel raises to maximal claudication fatigue, or work to maximal claudication fatigue.

The chemical entities described herein are useful for treating subjects with disorders that increase muscle fatigue. Such disorders may include, for example, peripheral artery disease, claudication, and muscle ischemia.

In peripheral vascular disease, vascular insufficiency results in diminished blood flow to tissues downstream of an obstruction leading to claudication (muscle pain during activities such as walking or stair climbing). Since claudication is the result of insufficient arterial blood delivery to meet the metabolic demands of working muscles that results in muscle ischemia and fatigue, fast skeletal troponin activators can be used to ameliorate fatigue induced by such vascular insufficiency. Thus, in some embodiments, the method comprises administering to a subject suffering from peripheral vascular disease or claudication an effective amount of a skeletal muscle troponin activator. In some embodiments, the skeletal muscle troponin activator improves resistance to skeletal muscle fatigue in the subject suffering from peripheral vascular disease or claudication.

Also provided are methods for enhancing fast skeletal muscle efficiency in a patient suffering from heart failure, comprising administering to said patient an effective amount of a skeletal muscle troponin activator as described herein that selectively binds the troponin complex of fast skeletal muscle fiber or sarcomere. In some embodiments, the skeletal muscle troponin activator as described herein activates fast skeletal muscle fibers or sarcomeres. In some embodiments, administration of a skeletal muscle troponin activator as described herein results in an increase in fast skeletal muscle power output. In some embodiments, administration of a skeletal muscle troponin activator as described herein results in increased sensitivity of fast skeletal muscle fibers or sarcomeres to calcium ion, as compared to fast skeletal muscle fibers or sarcomeres untreated with the compound. In some embodiments, administration of a skeletal muscle troponin activator as described herein results in a lower concentration of calcium ions causing fast skeletal muscle myosin to bind to actin. In some embodiments, administration of a skeletal muscle troponin activator as described herein results in the fast skeletal muscle fiber generating force to a greater extent at submaximal levels of muscle activation. In any of these embodiments, the skeletal muscle troponin activator may be a fast skeletal muscle troponin activator.

Also provided is a method for increasing time to fast skeletal muscle fatigue in a patient suffering from heart failure, comprising contacting fast skeletal muscle fibers with a skeletal muscle troponin activator that selectively binds to the troponin complexes of the fast skeletal muscle fibers. In some embodiments, the skeletal muscle troponin activator binds to form ligand-troponin-calcium ion complexes that activate the fast skeletal muscle fibers. In some embodiments, formation of the complexes and/or activation of the fast skeletal muscle fibers results in enhanced force and/or increased time to fatigue as compared to untreated fast skeletal muscle fibers contacted with a similar calcium ion concentration. In any of these embodiments, the skeletal muscle troponin activator may be a fast skeletal muscle troponin activator.

The chemical entities described herein are administered at a therapeutically effective dosage, e.g., a dosage sufficient to provide treatment for the disease states previously described. While human dosage levels have yet to be optimized for the chemical entities described herein, generally, a daily dose ranges from about 0.05 to 100 mg/kg of body weight; in certain embodiments, from about 0.10 to 10.0 mg/kg of body weight, and in certain embodiments, from about 0.15 to 1.0 mg/kg of body weight. Thus, for administration to a 70 kg person, in certain embodiments, the dosage range would be about from 3.5 to 7000 mg per day; in certain embodiments, about from 7.0 to 750.0 mg per day, and in certain embodiments, about from 10.0 to 100.0 mg per day. The amount of the chemical entity administered will, of course, be dependent on the subject and disease state being treated, the severity of the affliction, the manner and schedule of administration and the judgment of the prescribing physician; for example, a likely dose range for oral administration would be from about 70 to 700 mg per day, whereas for intravenous administration a likely dose range would be from about 70 to 750 mg per day depending on compound pharmacokinetics. In certain embodiments, the dose range is about 200-750 mg per day, or about 300-600 mg per day. Specific dosage amounts include 250, 300, 350, 400, 450, 500, 550, 600 and 750 mg per day. In further embodiments, the chemical entity is administered in an amount sufficient to maintain a mean plasma concentration of at least about 5 μg/ml for 24 hours, or, alternatively, 10 μg/ml, 12 μg/ml, 14 pg/ml, 16 μg/ml, or 20 μg/ml for 24 hours.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutically acceptable compositions include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The compounds and compositions described and/or disclosed herein may be administered alone or in combination with other therapies and/or therapeutic agents useful in the treatment of a disease or disorder.

The compounds and compositions described and/or disclosed herein may be combined with one or more other therapies to treat heart failure. Suitable additional therapeutics include digoxin, omecamtiv mecarbil, antiplatelet drug therapy such as, aspirin, ticlopidine, and clopidogrel; beta blocker therapy such as metoprolol or carvedilol; ACE inhibitors (i.e. inhibitors of angiotensin-converting enzyme) such as perindopril, captopril, enalapril, lisinopril, and ramipril; diuretics such as ethacrynic acid, torsemide, bumetanide, hydrochlorothiazide, acetazolamide, methazolamide, spironolactone, potassium canreonate, amiloride, and triamterene; calcium channel blockers such as amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, verapamil, diltiazem, mibefradil, bepridil, fluspirilene, and fendiline; statins such as atorvastatin, cerivastatin, fluvastatin, lovastatin, mevastatin, pitavastatin, pravastatin, rosuvastatin, and simvastatin; aldosterone antagonists such as eplerenone, canrenone, prorenone, and mexrenone; and angiotensin II receptor antagonists such as losartan, candesartan, valsartan, irbesartan, telmisartan, perosartan, olmesartan, and azilsartan. Other suitable additional therapies include angioplasty, stenting, or surgery (e.g., bypass surgery or surgery to remove an atherosclerotic plaque).

The above therapeutic agents, when employed in combination with the compounds and compositions disclosed and/or described herein, may be used, for example, in those amounts indicated in the Physicians' Desk Reference (PDR) or as otherwise determined by one of ordinary skill in the art.

The above therapeutic agents, when employed in combination with the compounds and compositions disclosed and/or described herein may be administered sequentially, simultaneously, or in various combinations. For example, administration of compositions of the disclosure is “A” and the additional therapeutic is “B,” exemplary combinations include A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, A/A/B/A, and the like.

The following examples serve to more fully describe the disclosed compounds the methods. It is understood that these examples in no way serve to limit the true scope of this invention, but rather are presented for illustrative purposes.

### EXAMPLE 1

### Effect of a Fast Skeletal Muscle Troponin Activator on Isometric Tension in Rat FDB Muscle Live Fibers

The fast skeletal troponin activator 6-ethynyl-1-(pentan-3-yl)-1H-imidazo[4,5-b]pyrazin-2-ol (Compound A) selectively sensitizes fast skeletal muscle to calcium ions by binding to the sarcomeric troponin complex and slowing the rate of Ca²⁺ release from troponin C. At the biochemical level, Compound A addition to fast skeletal myofibrils results in a leftward-shift of the myosin ATPase relationship to Ca²⁺ concentration. Compound A has little or no effect in myofibrils from slow skeletal and cardiac muscle illustrating its selectivity profile for fast skeletal muscle. Isothermal titration calorimetry has further confirmed a direct interaction of Compound A with fast skeletal troponin (K_{D}=40 nM). In chemically ‘skinned’ human vastus lateralis fibers from muscle biopsies (with the plasma membranes made freely permeable to Ca¹⁺), treatment of fast fibers with Compound A dramatically left-shifts the plot of the force-calcium relationship without increasing the maximum force or the shape of the curve. Skinned fast skeletal muscle fibers from rabbit psoas muscle show similar fiber-type selectivity and leftward-shift of the force-calcium relationship. The leftward-shift of the force-calcium relationship of muscle fibers and a corresponding shift in the force-frequency relationship of nerve-muscle pairs in situ demonstrate that Compound A increases muscle force at sub-maximal nerve stimulation rates and sensitizes fast skeletal muscle to Ca²⁺ (A. J. Russell et al. Nature Medicine, 2011; 378:667-75).

Adult male Sprague-Dawley rats (Charles River) between 250 and 300 g were anesthetized with a mixture of isoflurane gas and oxygen and quickly euthanized by cardiac excision. Hind feet were quickly removed at the ankle and placed in oxygenated Krebs solution at 4° C. (composition: 1 mM NaH₂PO₄, 5 mM KCl, 2 mM CaCl₂, 1 mM MgSO₄, 137 mM NaCl, 11 mM glucose and 1 mM NaHCO₃). Feet were then pinned out in fresh oxygenated Krebs buffer at room temperature and the skin from the sole of the foot removed with scissors. In rats, a small branch of the main flexor digitorum brevis (FDB) muscle extends from the heel of the foot to the little digit. This was dissected free with small scissors and the tendons at each end of the muscle were cut. The muscle was pinned out in Krebs solution and the surrounding fascia removed. Silk thread was tied with a small loop and then knotted on to the end of each tendon, creating a silk loop at each end of the muscle. This was then hooked on to the fixed lever arm and force transducer of an 801A in vitro analysis system (Aurora Scientific, Ontario, Canada) and perfused with Krebs solution at 30° C.

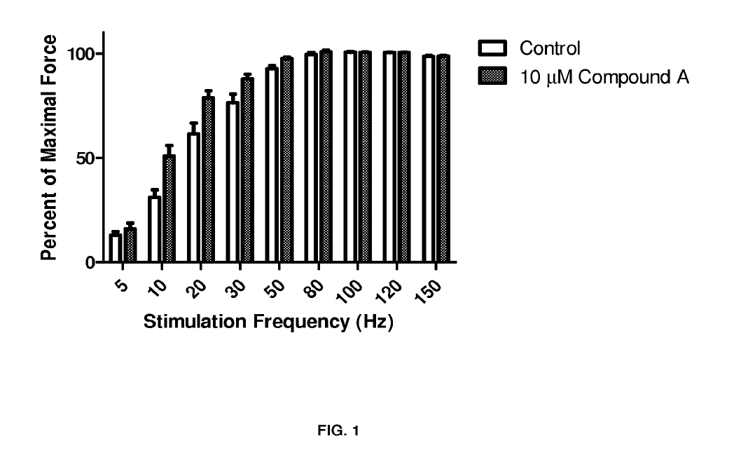

The effect of 10 μM of Compound A on the force/frequency relationship was measured. As shown in FIG. 1, Compound A increased sub-maximal force development of rat FDB muscle in vitro (mean specific tension +/− S.D.; *p<0.05 vs. baseline; n=6).

### EXAMPLE 2

### Effect of a Fast Skeletal Muscle Troponin Activator on Fatigue of Isometric Tension in Rat FDB Muscle Live Fibers

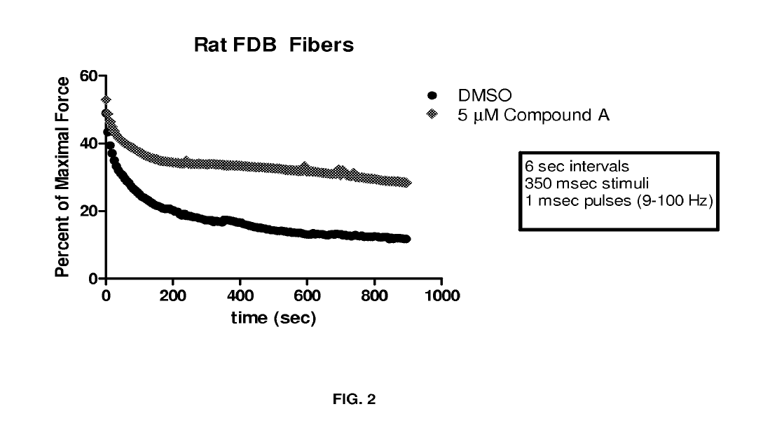

The isometric fatigue protocol was based on published studies (Germinario et al, 2004). Isolated rat FDB muscles were incubated at 4° C. with either 0.1% DMSO Krebs buffer or buffer containing 5 μM Compound A for 30 minutes. The tissues were then transferred to an isometric force transducer at 30° C. with the same concentration of DMSO or Compound A. Muscles were stimulated via field electrodes with supra maximal voltage to tetanus (120 Hz stimulation, 1 ms pulses, 350 ms duration) every minute and the length adjusted to achieve maximal tension development (Lₒ) which was recorded. The stimulation frequency was adjusted to achieve 50% of maximal force for each tissue: The average stimulation frequency required to achieve FMax 50% (mean +/− sd) for 0.1% DMSO was: 32.4 +/− 3.3 Hz, and for 5 μM Compound A it was 20.5 +/− 4.9 Hz. The muscles were then stimulated every six seconds for 15 minutes with field electrodes (1 ms stimulus, 350 ms trains) which produced a rapid drop in developed force over the course of 900 seconds for both the control fibers and Compound A fibers, with the control group showing a greater and more rapid drop in tension than the Compound A group. FIG. 2 shows that the average maximal force (Fmax) of control fibers fell to 24.38±4.3% of initial tension (11.8±1.9% of fMax) (lower plot) whereas Compound A only fell to 53.9±2.1% of initial tension at 900 seconds (28.4±1.2% f Max) (upper plot).

### EXAMPLE 3

### Effect of a Fast Skeletal Muscle Troponin Activator on Isometric Tension Relaxation Time in Rat EDL Muscle in situ

As with the in vitro FDB muscle studies, another predominately fast muscle fiber type, the extensor digitorum long us (EDL), was stimulated toward contractions in unconscious rats in situ. In these isometric muscle studies (measuring force at fixed length), force, time to peak contraction, time to half relaxation after cessation of stimulation (RT_{1/2}) and baseline tension were determined. These studies have the advantage that the nerve and muscle pair were intact and had typical blood flow to the muscle under investigation.

Rats were placed under anesthesia using isoflurane and the skin around the experimental leg was removed. The distal end of the EDL muscle and its associated tendon were then isolated. The rat was then placed on the platform of an Aurora in-situ muscle analysis rig (806C), maintained at body temperature via a circulating water system. The knee was immobilized in a clamp between two sharpened screws and the distal tendon cut and tied to the arm of a force transducer (Aurora Scientific, Ontario, Canada) using a silk suture. The muscle was stimulated directly via the peroneal nerve. For isolation of the nerve, a 1 cm incision was made at the upper thigh and the overlying gastrocnemius muscle was cut to expose an approximate 5 mm stretch of the peroneal nerve. This was then dissected free of surrounding connective tissue and a pair of stainless steel needle electrodes (0.10 mm) were hooked around the exposed nerve. Muscle contractile properties were assessed by applying an electrical current to the nerve and recording the force generated by the muscle via a servomotor. The muscle length was adjusted to produce the maximum isometric force (Lₒ) after sub-maximal stimulation (30 Hz, 1 ms pulses, 350 ms train duration). Once Lₒ had been established, the nerve was stimulated every 2 minutes with a 30 Hz train (1 ms stimuli, 350 ms duration) for the course of the experiment. This preparation was stable for 4-6 hours.

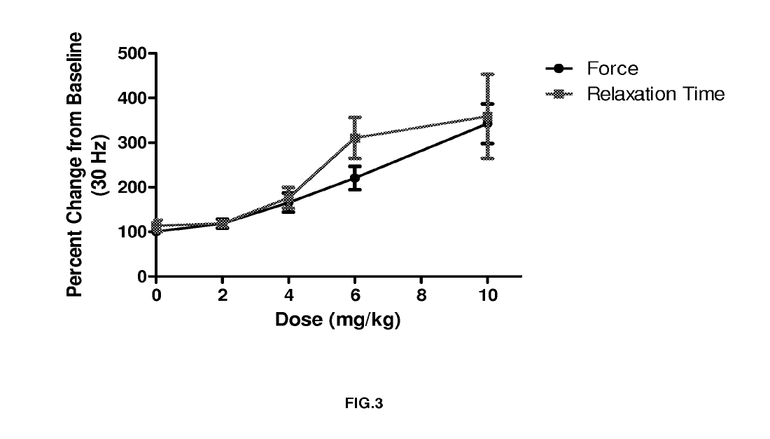

Once the length of the muscle was adjusted and a steady baseline force was achieved, solutions of Compound A (50% PEG300/10% EtOH/40% cavitron formulation) were administered via a femoral artery catheter as a single slow bolus over a 2 min period. Dose escalation was commonly carried out up to 10 mg/Kg with a maximal dosage volume of 5 ml/Kg. Treatment with Compound A resulted in an increase in sub-maximal force without increasing maximal force, similar to changes in force development observed in FDB muscles in vitro. The in situ relaxation time following arterial infusion of tirasemtiv revealed proportional changes in relaxation time with force up to a dose of 10 mg/kg (FIG. 3). These studies confirmed that the skeletal muscle troponin activator Compound A activated sub-maximal skeletal muscle force in situ with a similar pattern of activity to studies of FDB muscle fibers in vitro and demonstrated a corresponding increase in relaxation time by approximately 3.5-fold at 10 mg/kg doses.

### EXAMPLE 4

### Effect of a Fast Skeletal Muscle Troponin Activator on Fatigue in Rat EDL Muscle in situ

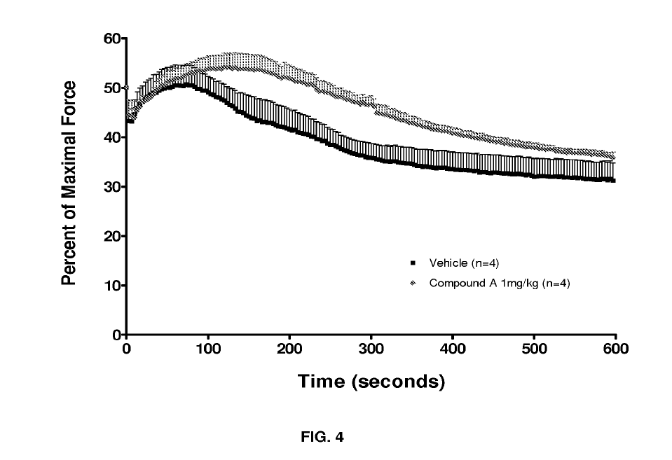

With the rat in situ EDL muscle preparation described in Example 3, a fatiguing protocol was utilized where muscle was stimulated for 600 seconds. In vehicle-treated rats EDL muscle was electrically stimulated via the peroneal nerve at 30 Hz. Because Compound A reduces the necessary stimulation frequency to achieve the same isometric tension, the peroneal nerve stimulation frequency was reduced in Compound A treated (1 mg/kg) rats to ensure similar force production to pre-dose levels (the average stimulation frequency of approximately 26 Hz was utilized for Compound A treated rats). Compound A or vehicle was delivered via duodenal cannula. To elicit muscle fatigue, the EDL muscle was stimulated with 350 msec electrical trains every 3 seconds for ten minutes at a frequency producing an initial force equal to 50% of maximal (F_{Max50}) as determined by the force-frequency relationship for each animal. The results, as summarized in FIG. 4, indicate that Compound A decreased rat EDL muscle fatigue in situ.

### EXAMPLE 5

### Effect of a Fast Skeletal Muscle Troponin Activator on Fatigue in Rat EDL Muscle Following Femoral Artery Ligation (FAL)

With the rat in situ EDL muscle preparation described in Example 3, a fatiguing protocol was utilized where muscle was stimulated for 600 seconds. Vehicle-treated muscle was electrically stimulated at 30 Hz while the stimulation frequency was reduced in Compound A treated rats to ensure similar force production to pre-dose levels, prior to femoral artery ligation (average stimulation frequency of 29 and 26Hz, for 0.5 mg/kg and 1 mg/kg Compound A, respectively). This protocol produced a robust and reproducible fatigue in EDL muscle following femoral artery ligation, with a transient increase to 136.0±6.8% of initial force over the first 90-100 seconds, followed by a rapid drop in force before stabilization at approximately 40% of initial force. The initial rise in tension is believed to be due to Pi-induced increases in free intracellular Ca²⁺ due to inhibition of sarco(endo)plasmic reticulum Ca²⁺-ATPases (SERCAs) pumping into the SR (Allen, Physiol Rev 88:287-332, 2008). Following ligation of the femoral artery (R. A. Challiss et al. Biochem J. 1986 Dec. 1; 240(2):395-401) Compound A was administered in solution (50% PEG300/10% EtOH/40% Cavitron formulation) via a jugular vein catheter as a single slow bolus over a 2 min period.

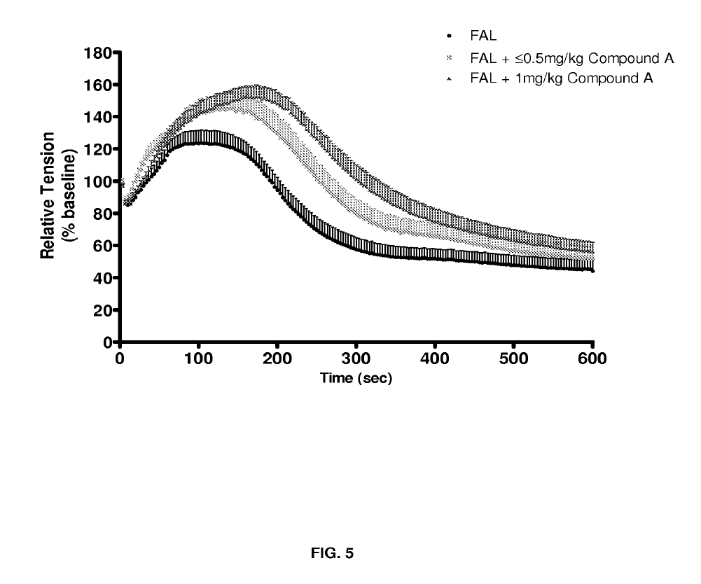

As shown in FIG. 5, treatment with Compound A produced a dose-dependent increase in the time to fatigue and tension-generating capacity in the FAL rats compared to vehicle-treated animals. Thus, the fatigue protocol in Compound A treated rats resulted in a longer rise to a greater initial increase in force to 156.3±10.4% of initial force over 160-170 seconds, compared to vehicle treated animals. Time for force to decrease to 50% of initial force was lengthened from 259±30 seconds to 752±64 seconds (P<0.0001, T-test).

### EXAMPLE 6

### Effect of Fast Skeletal Muscle Troponin Activators on Rat Plantor Flexor Force and Power in situ

Rats were placed under anesthesia using isoflurane and the sciatic nerve of the experimental leg exposed. The rat was then placed on the platform of an Aurora in-situ muscle analysis rig (806C), maintained at body temperature via a circulating water system. The knee was immobilized in a clamp between two sharpened screws and the foot attached securely to the footplate of a force transducer (Aurora Scientific, Ontario, Canada) using laboratory tape. The muscle was stimulated directly via the sciatic nerve. For isolation of the nerve, a 1 cm incision was made at the upper thigh and the overlying muscle was dissected to expose an approximate 5 mm stretch of the sciatic nerve. This was then dissected free of surrounding connective tissue and a pair of stainless steel needle electrodes (0.10 mm) were hooked around the exposed nerve. The peroneal branch was severed to remove innervation to the plantar-extensor muscle groups. Muscle contractile properties were assessed by applying an electrical current to the nerve and recording the force generated by the muscle via a servomotor. Isokinetic contractile properties were assessed as force generated during a pre-programmed movement of the footplate. Footplate movements were 0.7 radians in size (40° C.).

Solutions of Compound A (50% PEG300/10% EtOH/40% cavitron formulation) were administered via a femoral vein catheter as a single slow bolus over a 2 min period, with a maximal dosage volume of 5 ml/Kg. During the experiment, blood was drawn via the tail vein for compound concentration analysis. At the end of each assay, the length and weight of the muscle was recorded, and measured force normalized to the mass of the muscle (N/g).

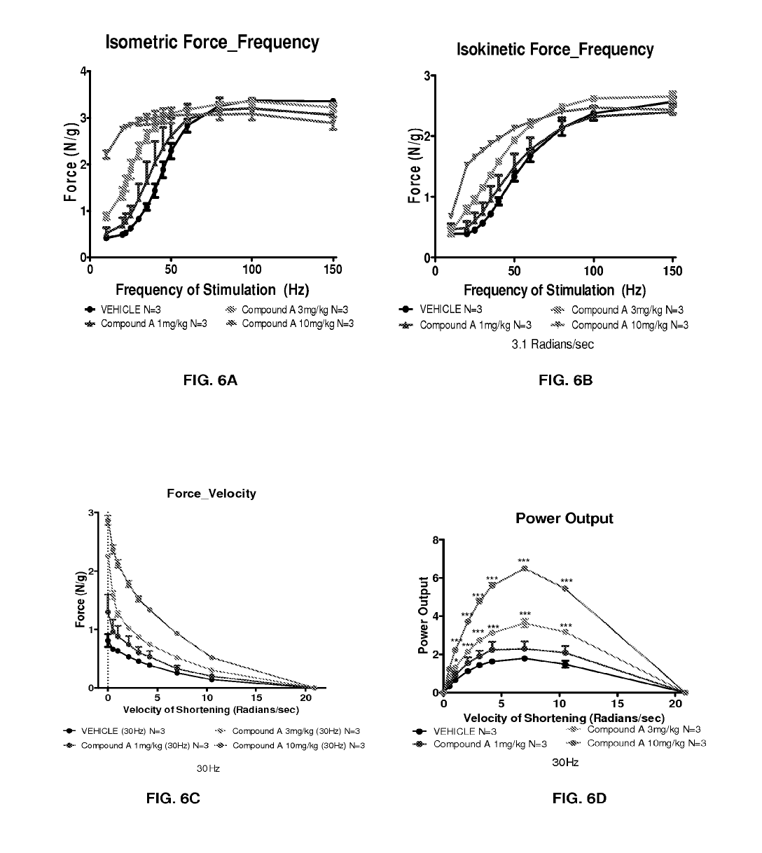

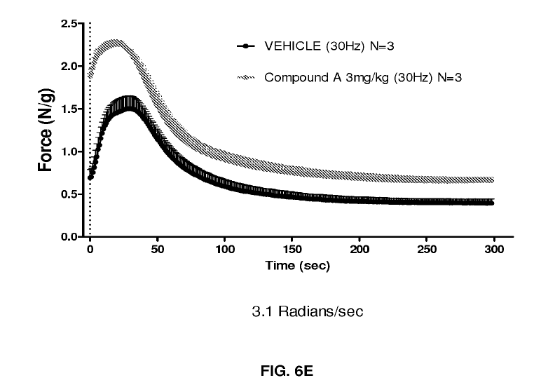

The results are summarized in FIGS. 6A-6D. As shown in FIG. 6A, the isometric force frequency relationship for rat plantarflexor muscles increased in the submaximal range in a dose dependent manner following administration of Compound A. As shown in FIG. 6B, the isokinetic force frequency relationship (at 3.1 radians/s) increased in the submaximal range in a dose dependent manner following administration of Compound A. As shown in FIG. 6C, the force-velocity relationship at 30 Hz increased across all velocities in a dose and velocity dependent manner. As shown in FIG. 6D, the power output corresponding to the force velocities in FIG. 6C displayed a dose dependent increase, while maintaining similar maximum power characteristics. FIG. 6E shows force generation during an isokinetic fatigue protocol of 1 flexion per second at 3.1 radians/s with 0.7 radian displacement and 30 Hz stimulation frequency. Compound A increased force generation throughout the curve to generate a total of 55% more work over the 300 second period, while maintaining a similar profile to that of the vehicle.

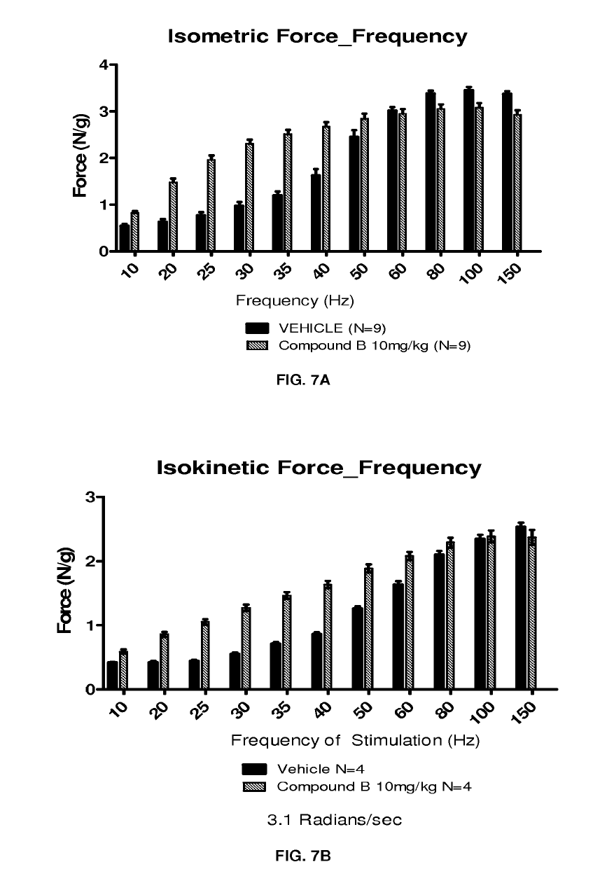

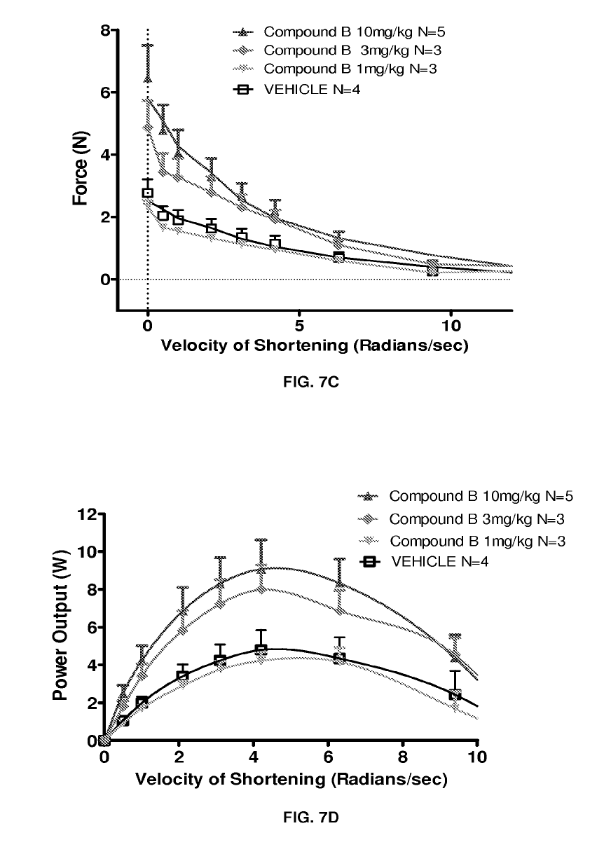

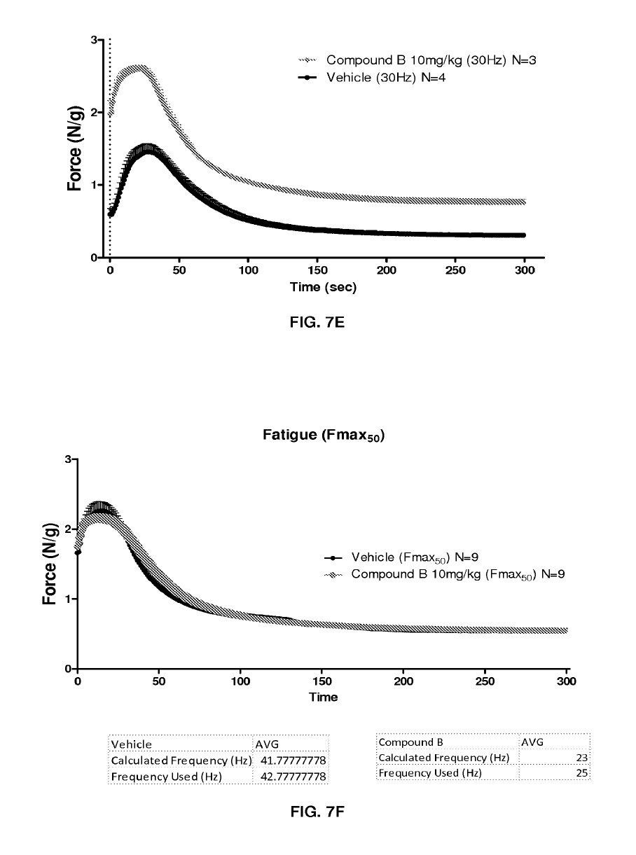

The same protocol was repeated with a second skeletal muscle troponin activator, 1-((1R)-1-methylpropyl)-6-chloro-7-pyrazolylimidazo[4,5-b]pyridin-2-ol (Compound B). Compound B and analogous skeletal muscle troponin activators are disclosed in U.S. Pat. No. 7,989,469. The results are summarized in FIGS. 7A-7F. As shown in FIG. 7A, the isometric force frequency relationship for rat plantarflexor muscles increased in the submaximal range in a dose dependent manner following administration of Compound B. As shown in FIG. 7B, the isokinetic force frequency relationship (at 3.1 radians/s) increased in the submaximal range in a dose dependent manner following administration of Compound B. As shown in FIG. 7C, the force-velocity relationship at 30 Hz increased across all velocities in a dose and velocity dependent manner. As shown in FIG. 7D, the power output corresponding to the force velocity curves in FIG. 7C displayed a dose-dependent increase, while maintaining similar maximum power characteristics. FIG. 7E shows force generation during an isokinetic fatigue protocol of 1 flexion per second at 3.1 radians/s with 0.7 radian displacement and 30 Hz stimulation frequency. Compound B increased force generation throughout the curve to generate a total of 105% more work over the 300 second period, while maintaining a similar profile to that of the vehicle. FIG. 7F shows force generation during an isokinetic fatigue protocol of 1 flexion per second at 3.1 radians/s. 0.7 radian displacement and stimulation frequency calculated to provide 50% of maximum isokinetic tension. Compound B maintained force generation throughout the curve, at almost 50% of vehicle stimulation frequency, to generate the same total work over the 300 second period, while maintaining a similar profile to that of the vehicle.

### EXAMPLE 7

### Effect of a Fast Skeletal Muscle Troponin Activator on Cage Grid Hang Time in Healthy Rats

Static fatigue in conscious rats was assessed by measuring the length of time that healthy female rats would hang upside down from a cage grid. Rats were trained to hang upside-down from a cage grid and the time to drop to soft padding below was recorded. Baseline hang-times were recorded for each animal (n=24) for a two week period of time. The rats were then treated with Compound A (200 ppm) in chow for two weeks while hang times were monitored daily. Finally, the rats were withdrawn from Compound A in their chow for five days while hang times were recorded daily.

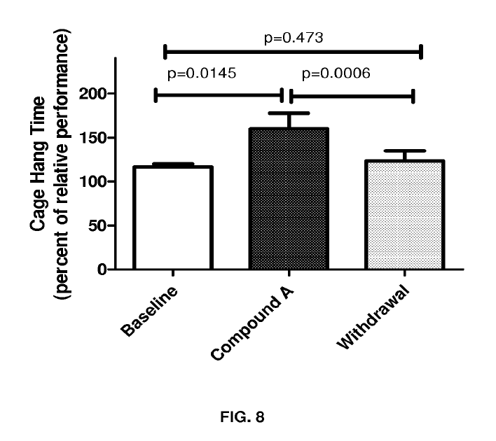

The mean cage hang performance in the rats over the baseline period showed a significant improvement in their ability to hang upside-down. Thus, hang-time performance at the end of the baseline period was significantly greater than at the initiation of the baseline period when comparing individual performance (the final three days of baseline testing yielded an increase to 116±4% mean±sem, of baseline performance for control rats; 618 +/− 65 sec). As shown in FIG. 8, rats fed chow containing Compound A (200 ppm) over a two week period increased their grid hang time from 116% to160±18% for the average final three days of Compound A dosing compared to baseline (p<0.02 by paired T-test; 899 +/− 157 secs). Withdrawal of Compound A for five days led to a decrease in hang-time performance in the majority of animals, as measured by normalized performance (average final three days of five day period=124±12%, p<0.001 by paired T-test; 698 +/− 122 sec).

### EXAMPLE 8

### Effect of a Fast Skeletal Muscle Troponin Activator on Rotarod Running Assay in Healthy Rats

Female Sprague Dawley rats (210-260 g) were obtained from Charles River Laboratories and acclimated in the test facility for a minimum of six days prior to the start of the study. All rats were trained the day prior to compound administration. Training consisted of placing the rats on the rotating drum (rod), starting at a low constant speed (10 RPM). The rats were acclimated to walk on the drum for 5 minutes before resting. A second training session of an increasing speed from 14-16 RPM was initiated after all rats in the experimental group had finished the first training session. Those rats that failed to run during the course of the training were removed from the experiment. On the day of the experiment animals were dosed thirty minutes prior to start of test. The test began with a 5 minute primer session, whereby animals were run at an increasing speed from 14-16 RPM over 5 minutes. Rats were then run at a constantly accelerating rate from 12 RPM to 25 RPM over the course of 10 minutes. Once 25 RPM had been reached, a constant speed of 25 RPM was maintained for an additional 5 minutes. Time to fall was recorded, with the test being terminated at 900 seconds.

Compound A was administered via oral gavage 30 minutes prior to assessment. Each dose was formulated as a suspension containing 0.2% Tween 80, 0.5% HPMC and water. Dose volume was 5 mL/kg. Vehicle (0.2% Tween 80, 0.5% HPMC and water) was administered similarly. Control treatments were chosen based on association with amelioration of central fatigue (caffeine, Davis 2003), muscular fatigue (creatine, Boyadjiev, 2007) and dual central/muscular fatigue (phosphoserine, Fanelli 1976). Creatinine (300 mg/kg), caffeine (10 mg/kg) and phosphoserine (1000 mg/kg) were administered in water by oral gavage 60 min, 30 min and 24 hours prior to test respectively.

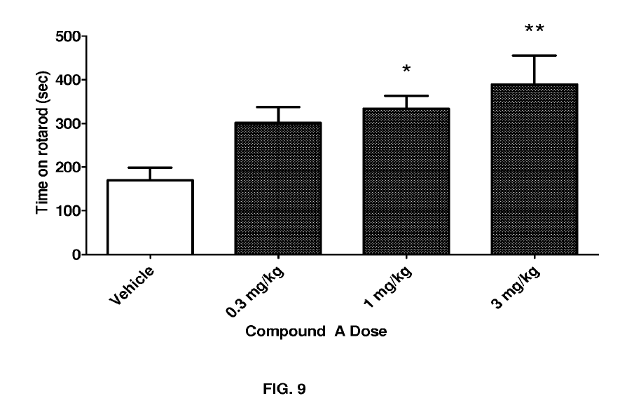

As shown in FIG. 9 and in Table 1 below, rats administered Compound A showed a dose-dependent increased in running time on a slowly accelerating rotarod, with 3 mg/kg dose showing more than a doubling of running time at maximum dose tested. Rats administered creatine, caffeine and phosphoserne (compounds previously shown to improve performance in other exercise assays) showed no significant difference.

TABLE 1

| | Run time (s) | |
|---|---|---|
| Dose (mg/kg) | mean ± SEM | P-value |
| Vehicle | 169 ± 28 | N/A |
| 0.3 | 301 ± 36 | NS |
| 1 | 334 ± 29 ( ) | p < 0.05 |
| 3 | 389 ± 65 ( ) | p < 0.01 |

### EXAMPLE 9

### Effect of a Fast Skeletal Muscle Troponin Activator on Treadmill Running Assay in Healthy Rats

Male Sprague Dawley rats (Charles River), 10-12 weeks old, 250-400 g. Rats were acclimated for a minimum of 2 days and weight was measured weekly. The endurance capacity of rats was assessed using a progressive exercise test as previously described (A. Aaker et al. J Cardiovasc Pharmacol28: 353-362, 1996; B. Helwig et al. J Appl Physiol. 2003 June; 94(6):2225-36). After familiarization with the treadmill apparatus, rats were run at a treadmill speed of 30 meters per minute (m/min) with a 5% incline. Every 15 minutes, the treadmill speed was increased by 5 meters per minute and the rats continued to exercise until they reached the point of fatigue and were unable to continue exercising (FIG. 1A). Exercise time was measured in minutes while exercise distance was recorded in meters. Compound A was administered via oral gavage 2 hours prior to assessment. Each dose was formulated as a suspension containing 1% hydroxypropyl methylcellulose (HPMC), 0.2% Tween 80, and micronized Compound A, and dose volume was 5 ml/kg. Vehicle (0.2% Tween 80, 1% HPMC and water) was administered similarly.

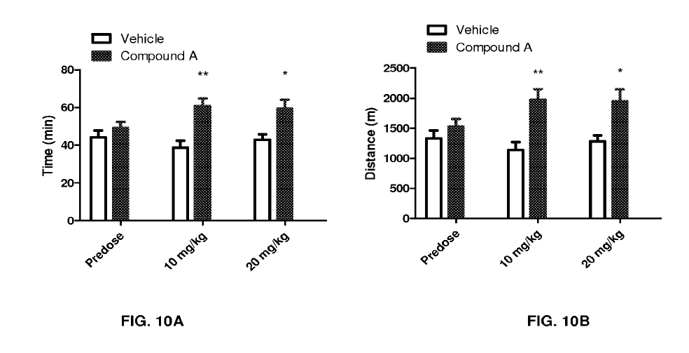

Doses of 10 and 20 mg/kg of Compound A resulted in an increase in treadmill running time of 20% over baseline and 50% over vehicle control (FIG. 10A). Equivalent increases were seen in distance run (FIG. 10B). These results are tabulated in Table 2 below.

TABLE 2

| | Distance (m) | | | Time (Min) | | |
|---|---|---|---|---|---|---|
| | Mean ± SEM | | | Mean ± SEM | | |
| | Compound A | Vehicle | P-value | Compound A | Vehicle | P-value |
| Predose | 1527 ± 127 | 1330 ± 134 | NS | 49.3 ± 3.2 | 44.2 ± 3.6 | NS |
| 10 mg/kg | 1972 ± 176 | 1140 ± 129 | <0.01 | 60.7 ± 4.2 | 38.9 ± 3.6 | <0.01 |
| 20 mg/kg | 1943 ± 205 | 1281 ± 101 | <0.05 | 59.4 ± 4.7 | 42.9 ± 2.8 | <0.05 |

### EXAMPLE 10

### Bilateral Concentric Heel Raise Test

Patients with peripheral artery disease (PAD) and claudication experience reproducible symptoms of leg pain during walking exercise. The symptom of claudication is due to the exercise-induced ischemia-perfusion mismatch of the muscles in the legs. Claudication pain is most commonly experienced in the calf muscles, limiting both walking distance and functional exercise capacity. Peak exercise performance measured as maximal walking time during a standardized graded treadmill test is the gold standard for assessing functional exercise capacity in PAD and is often used as the primary endpoint in clinical trials. Because of the local metabolic and hemodynamic perturbations in patients with claudication, it was assumed that a test of repeated bilateral heel raises would: 1) elicit leg claudication pain symptoms and 2) provide a functional assessment for symptom-limited muscular strength and fatigue. This experiment demonstrates the utility and reproducibility of a novel heel raise test of muscle function and claudication-limited exercise performance in patients with PAD and claudication.

Objectives of the study included: 1) to determine the baseline characteristics and variance of the bilateral heel raise test among patients with PAD; and 2) to determine the variance and intra-class correlation coefficients of heel raise test parameters among three repeated baseline measurements

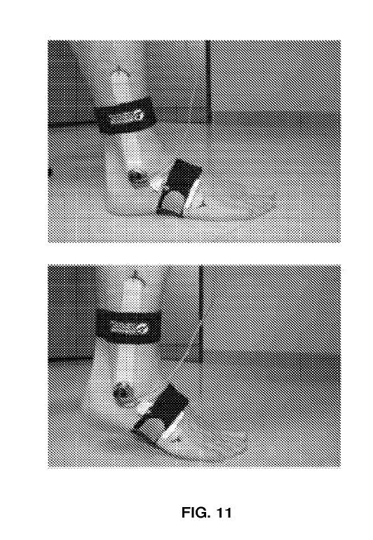

As part of a multi-center trial, the bilateral heel raise test was employed to assess symptom-limited muscle strength and fatigue at three visits, each separated by 1 week. Test instrumentation consisted of an electro-mechanical goniometer, handheld data processor, personal computer, and automated data collection software. The lateral aspect of the ankle on the dominant leg was instrumented with an electro-mechanical goniometer to assess ankle angle position and range of motion (Noraxon U.S.A., Inc., Scottsdale, Ariz.) (FIG. 11). Ankle plantar flexion was monitored and recorded using the goniometer handheld processor connected to a PC-based data collection system. Patients were positioned standing in a clinic doorway and instructed to perform heel raises at the frequency as directed by a metered, audible cue (1 heel raise every other second˜0.5 Hz). Subjects reported the onset of claudication symptoms, and the test was performed to intolerable/maximal claudication pain and fatigue. The total number of heel raises, time, and a calculated index of work performed were assessed from the beginning of test to the onset of claudication and to maximal exercise. An index of work performed was calculated: Heel Raise Work Index (HRWI)=(sine*foot length)*body mass. The number of heel raises were defined as the number of heel raises achieving or exceeding 20 degrees of ankle plantar flexion. A mixed-effects model was employed (fixed effect of Visit and random intercepts for patients) to determine the intra-class coefficient (ICC) and evaluate potential differences among pre-treatment means of the repeated heel raise tests.

The demographics and intra-class correlation coefficients of the study are shown in the tables below.

Demographics
N = 61
*Mean ± SD unless noted

| | |
|---|---|
| Age (Yrs) | 67.3 ± 9.2 |
| Weight (Kg) | 76.96 ± 17.30 |
| Currently smoking (%) | 39.3% |
| Male (%) | 85.2% |

Intra-class Correlation Coefficients

| | |
|---|---|
| Time to Claudication Onset | 79.2% |
| Number of repetitions to claudication onset | 78.4% |
| Work performed to claudication onset | 72.2% |
| Time to intolerable claudication or calf muscle | 78.7% |
| fatigue | |
| Number of repetitions to intolerable | 76.3% |
| claudication or calf muscle fatigue | |
| Work performed to intolerable claudication or | 75.2% |
| calf muscle fatigue | |

The results of the heel raise test are tabulated below.

Heel Raise Test Parameter by Visit

| Least squares mean ± SE | Visit 1 | Visit 2 | Visit 3 | P-value* |
|---|---|---|---|---|
| Time to Claudication onset (s) | 44.2 ± 2.3 | 41.3 ± 2.3 | 44.2 ± 2.3 | 0.0972 |
| Number of repetitions to | 20.8 ± 1.1 | 20.7 ± 1.1 | 22.5 ± 1.1 | 0.0527 |
| claudication onset (#) | | | | |
| Work performed to claudication | 86.7 ± 5.0 | 85.9 ± 5.0 | 96.8 ± 5.2 | 0.0155 |
| onset (kg-m) | | | | |
| Time to intolerable claudication | 78.4 ± 6.0 | 70.9 ± 6.1 | 75.3 ± 6.2 | 0.1806 |
| or calf muscle fatigue (s) | | | | |
| Number of repetitions to | 36.0 ± 2.9 | 34.5 ± 2.9 | 36.9 ± 3.0 | 0.3361 |
| intolerable claudication or calf | | | | |
| muscle fatigue (#) | | | | |
| Work performed to intolerable | 146.01 ± 9.68 | 142.39 ± 9.74 | 153.09 ± 9.95 | 0.5261 |
| claudication or calf muscle | | | | |
| fatigue (kg-m) | | | | |

*Comparison of least squares means computed by mixed effect model for differences between visits.

This study shows that bilateral heel raises performed according to a specified protocol elicited claudication pain in test subjects and provided a functionally relevant, easy-to-deploy, and cost effective measure of calf muscle endurance and fatigue in patients with PAD. The parameters assessed from a single bilateral concentric heel raise test demonstrated reliability among baseline measurements across a 3-week period in patients with PAD and claudication. Thus, the bilateral concentric heel raise test can be used as a diagnostic tool for patients suffering from vascular diseases (such as PAD and/or claudication) and to determine the efficacy of drugs (e.g., skeletal muscle troponin activators) to treat the symptoms of disease, including skeletal muscle fatigue.

### EXAMPLE 11

### Use of Bilateral Heel Test to Evaluate the Effect of Skeletal Muscle Troponin Activators in Patients with Claudication

This study was a double-blind, randomized, placebo-controlled, three-period cross-over, hypothesis generating Phase II study in patients with peripheral artery disease and claudication. The primary objective of the study was to demonstrate an effect of single doses of a skeletal muscle troponin activator (Compound A) on measures of skeletal muscle function and fatigability. Secondary objectives included (a) evaluation and characterization of the relationship, if any, between the doses and plasma concentrations of Compound A and its pharmacodynamic effects, and (b) evaluation of the safety and tolerability of Compound A administered as single doses.

Key inclusion criteria for the study included: 1) Stable claudication for the last 6 months (Fontaine Stage II) in at least one calf muscle; 2) Peripheral artery disease: ankle-brachial index at rest <0.90 in at least one leg in which the patient experiences claudication; 3) Ability to perform the bilateral heel raise test to claudication-limited maximum muscle performance at a contraction frequency of once every other second; 4) Ability to complete a 6-Minute Walk Test.

Key exclusion criteria for the study included: 1) Fontaine Stage III-IV leg ischemia (rest pain, tissue necrosis or gangrene); 2) Leg, hip, or knee surgery within 6 months prior to randomization; 3) Within 3 months prior to randomization: a) any revascularization procedure (coronary or peripheral); b) life-threatening ventricular arrhythmias, unstable angina, stroke, and/or myocardial infarction; and c) NYHA Class III or IV heart failure; and 4) Screening Heel Raise Test and 6-Minute Walk Test not limited by claudication.

Single doses of each of 375 mg Compound A, 750 mg Compound A and placebo were administered in random order with a 6 to 10 day wash out between each dose. The protocol was amended after 33 patients due to adverse events in two patients at 750 mg; the remainder received 500 mg Compound A instead of 750 mg. Assessments include 1) Bilateral Heel Raise Test (as described below) using electrogoniometry at 3 and 6 hours after dosing, and 2) 6-Minute Walk Test at 4 hours after dosing. Results were analyzed using a repeated-measures ANCOVA with treatment, sequence, period, baseline, and patient in the model. In the event of model assumption violations, non-parametric methods were utilized

In the bilateral heel raise test, the lateral aspect of the ankle on the dominant leg was instrumented with an electromechanical goniometer connected to a PC-based data collection system to monitor and record ankle angle position and range of motion (Noraxon U.S.A., Inc., Scottsdale, Ariz.). Patients were instructed to perform heel raises at the frequency as directed by a metered, audible cue (1 heel raise every other second˜0.5 Hz) Patients reported the onset of claudication symptoms, and the test was performed to intolerable/maximal claudication pain and fatigue. The total number of heel raises, time, and a calculated index of work performed were assessed from the beginning of test to the onset of claudication and to maximal exercise. An index of work performed was calculated as follows: Heel Raise Work Index (HRWI)=(sinθ°*foot length)*body mass.

The patient population that participated in the study is described in the tables below.

Demographic Characteristics

| | | Mean (SD) or | |
|---|---|---|---|
| | | N (percent of total) | |
| Total N | | 61 | (100%) |
| Age (years) | | 67.3 | (9.2) |
| Sex: | Female | 9 | (14.8%) |
| | Male | 52 | (85.2%) |

| | | |
|---|---|---|
| BMI (kg/m²) | 26.4 | (3.6) |
| Smoking Status | | |
| Current | 24 | (39.3%) |
| Former | 35 | (57.4%) |
| Never | 2 | (3.3%) |
| Tobacco Use (units/day) | 14.7 | (11.0) |
| Race: | | |
| Asian | 1 | (1.6%) |
| Black | 7 | (11.5%) |
| White | 52 | (85.2%) |
| Other | 1 | (1.6%) |
| Ethnicity: | | |
| Hispanic | 7 | (11.5%) |
| Non-hispanic | 54 | (88.5%) |

Baseline Performance on Pharmacodynamic Outcome Measures

| | Mean (SD) |
|---|---|
| Time to claudication onsent (seconds) | 43.7 (17.9) |
| Time to end of test (seconds) | 78.4 (48.1) |
| Number of full repititions to claudication | 20.6 (8.6) |
| onset | |
| Number of full repititions completed to end | 36 (23.1) |
| of test | |
| Work index to claudication onset (kg-m) | 87.0 (39.1) |
| Work index total to end of test (kg-m) | 145.8 (74.3) |
| 6-minute walk total distance | 1078.8 (204.0) |

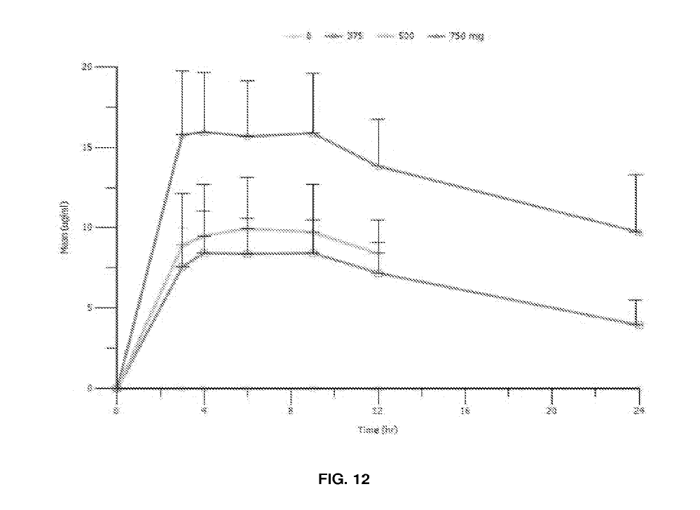

The results of the pharmacokinetics study are depicted in FIG. 12, which shows the mean (±SD) Compound A plasma concentrations over time. Mean plasma Compound A concentrations showed relatively dose proportional increases. Mean plasma concentrations remained within the pharmacologically active range throughout the 24-hour observation period, even at the 375 mg dose.

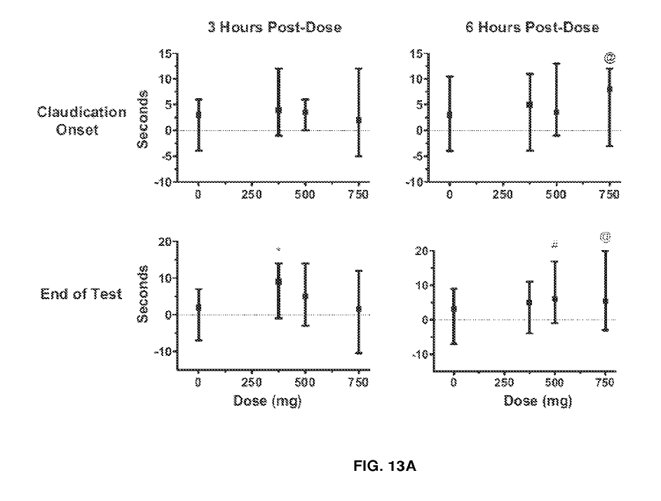

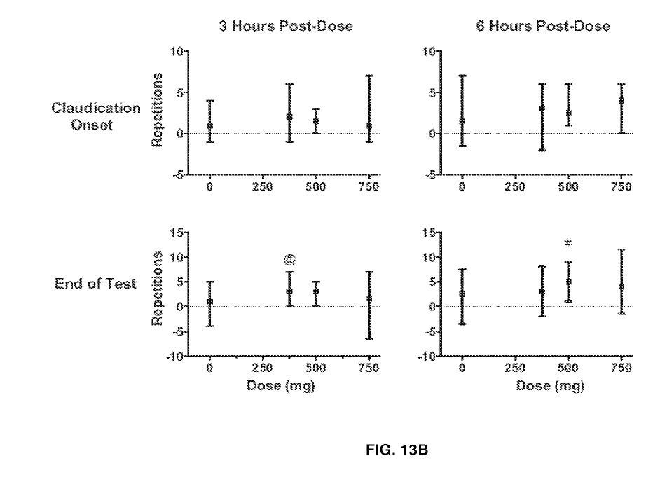

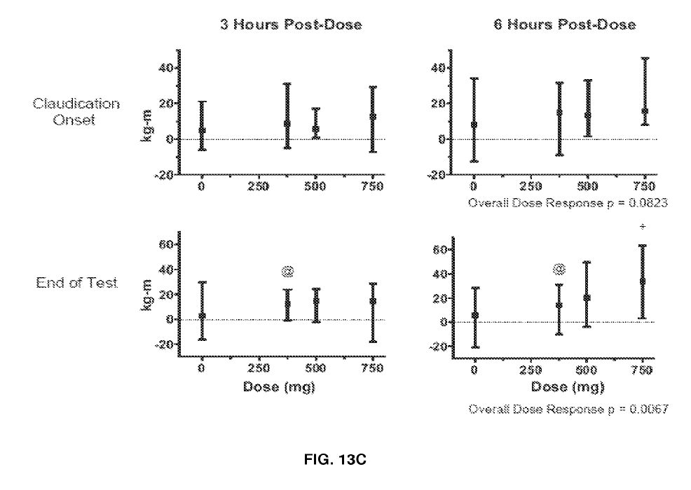

The results of the bilateral heel raise test are shown in FIGS. 13A-13C. FIG. 13A shows the time to onset of claudication or the end of the test (i.e., failure or intolerable claudication pain) for each of the three doses of Compound A at 3 and 6 hours post-dose. FIG. 13B shows the number of complete heel raise repetitions to onset of claudication or end of test for each of the three doses of Compound A at 3 and 6 hours post-dose. FIG. 13C shows the work done to onset of claudication and end of test for each of the three doses of Compound A at 3 and 6 hours post-dose. All values are represented as median ± interquartile range. (Symbols: @ p<0.10; # p<0.05; * p<0.01; + p<0.002).

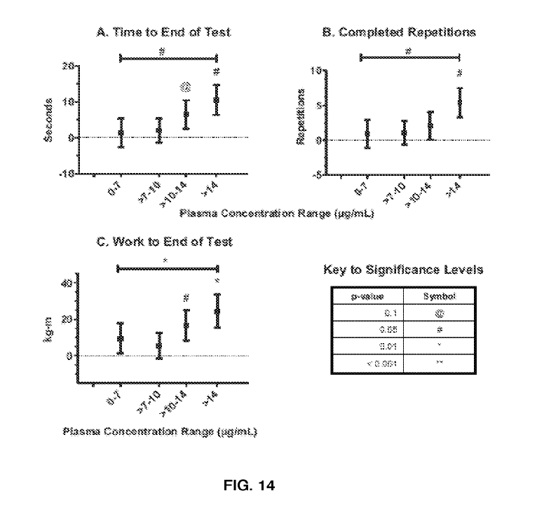

The PK/PD analysis shows a strong relationship between Compound A plasma concentrations and outcome (FIG. 14). Pharmacokinetic samples were obtained at the time of each Heel Raise Test. All measured plasma Compound A concentrations were divided into quartiles. The placebo corrected LS mean change from baseline ±SEM for the simultaneously obtained value of each outcome measure is plotted at the mid-point of each concentration bin. There was a strong positive relationship between Compound A plasma concentrations and all outcomes in the Heel Raise Test. Significance levels for individual comparisons to placebo are indicated on the table in the lower right-hand panel. Symbols above the horizontal bars on each graph in Compound A indicate the p-value for the slope of the concentration/response relationship.

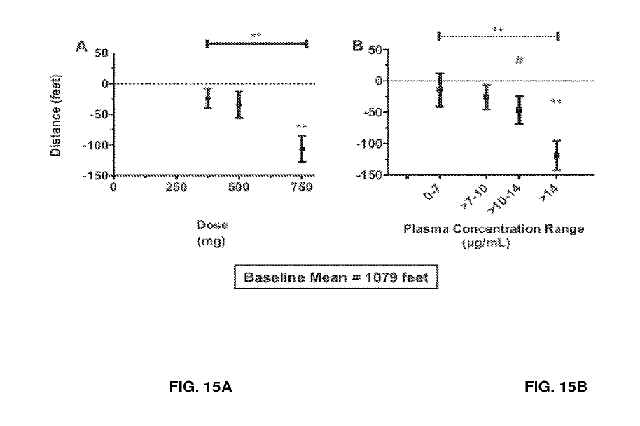

Compound A administration was associated with a dose and concentration dependent decrease in the distance patients traversed during a 6-Minute Walk Test (FIGS. 15A-15B). In FIG. 15A, values displayed are placebo-corrected LS mean changes from baseline ±SEM; ** p<0.0001 for overall dose response (indicated by horizontal bar over the figure) and for comparison of the 750 mg dose to placebo. In FIG. 15B all measured plasma Compound A concentrations were divided into quartiles. The placebo-corrected LS mean change from baseline ±SEM for the simultaneously obtained value of each outcome measure was plotted at the mid-point of each concentration range. ** p <0.0001 for overall concentration response (indicated by horizontal bar over the figure) and for comparison of the highest concentration range to placebo; # p<0.05 for comparison of the second highest range to placebo. Note that the placebo-corrected changes shown are small relative to the mean distance of 1079 feet traversed at the screening visit.

The results of these studies indicate that the fast skeletal muscle troponin activator Compound A increased calf muscle performance in patients with calf claudication as evidenced by heel raise testing. Both increases in muscle performance and adverse events appear related to increasing both dose and plasma Compound A concentration. Performance on 6-Minute Walk Test was inversely related to dose and Compound A plasma concentration. Dose related adverse events, particularly dizziness and others related to walking, may explain this negative effect on the 6-Minute Walk.

### EXAMPLE 12

### Effect of a Fast Skeletal Troponin Activator on Muscle Fatigue in Healthy Rats

The effect of the fast skeletal muscle troponin activator 1-(2-(((trans)-3-fluoro-1-(3-fluoropyridin-2-yl)cyclobutyl)methylamino)pyrimidin-5-yl)-1H-pyrrole-3-carboxamide (Compound C) on muscle fatigue in healthy rats was determined by a rotarod running time test. Compound C was administered to test rats at the doses between 0.3 mg/kg and 100 mg/kg (˜70 nM-28 μM). On the day of the assessment, the animals began by running at an increasing speed from 14-16 RPM over 5 minutes. Rats were then run at a constantly accelerating rate from 12 RPM to 25 RPM over the course of 10 minutes. Time to fall was recorded, with the test being terminated at 600 seconds. The plasma levels were determined at the end of the experiments (not at Cₘₐₓ). The plasma levels for the indicated doses were determined to be the following:

| Dose | [Plasma] |
|---|---|
| (mg/kg) | (μM) |
| 0.01 | 0.003 |
| 0.03 | 0.007 |
| 0.1 | 0.024 |
| 0.3 | 0.083 |
| 1 | 0.204 |
| 3 | 0.478 |
| 10 | 1.95 |
| 30 | 3.64 |
| 100 | 28.0 |

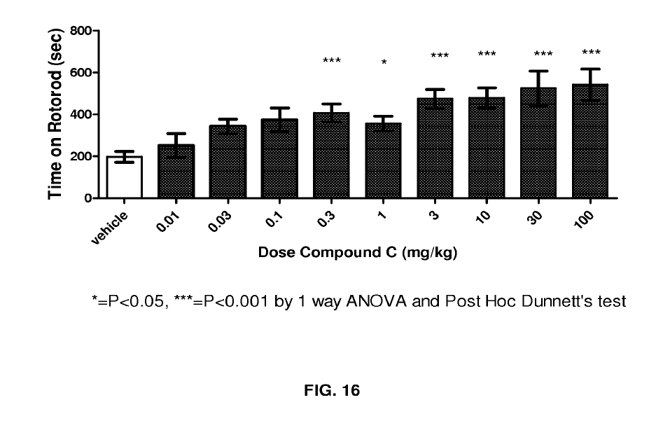

As shown in FIG. 16, significant increases were observed in running time in this fatigue model for Compound A-treated rats vs. vehicle controls between 0.3 mg/kg and 100 mg/kg. Thus, Compound A increases running time in the fatigue-rotarod test in healthy rats.

### EXAMPLE 13

### Effect of a fast skeletal troponin activator on rat heart failure model

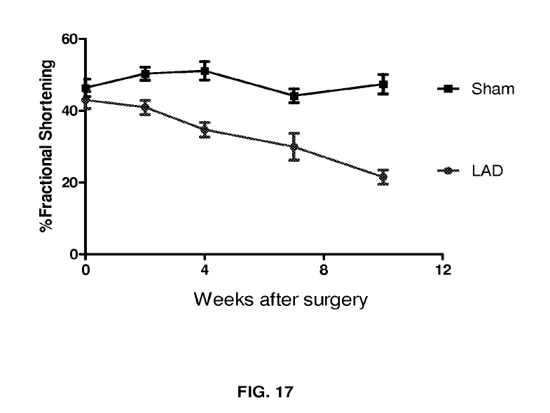

To determine the effect of Compound C on skeletal muscle function in patients with heart failure, a rat heart failure model was used. Female Sprague Dawley rats (<250 g) were obtained from Charles River Laboratories having the left anterior descending coronary artery ligated prior to shipping (LAD-HF rats). Sham operated controls were also obtained from the same surgical preparation (sham controls). All rats underwent assessment of cardiac function after a three day acclimation to provide baseline levels. Animals were assessed on weeks 4, 7 and 10 to observe the progress of exercise intolerance at least 3 days after echocardiography. Exercise performance was assessed using the fatiguing rotarod protocol described in Example 11 (5 minute run ramping from 14-16 RPM, followed by run time assessment during a 10 minute ramp from 12 to 25 RPM). LAD-HF rats were selected based on a left ventricular fractional shortening <25% and reduced run time compared to sham controls. As shown in FIG. 17, the heart failure phenotype of the LAD-HF rats developed over several weeks post-surgery. Decreases in fractional shortening (determined by echocardiography) were apparent.

Animals were assigned in a cross-over fashion whereby half of the animals received Compound C (10 mg/kg PO) and half received vehicle (19.3%PEG: 80% (15%) captisol, pH3, 0.2% tween, 0.5% HPMC) via oral gavage 30 minutes prior to rotarod assessment on Day 1 and then the opposite treatment on Day 2.

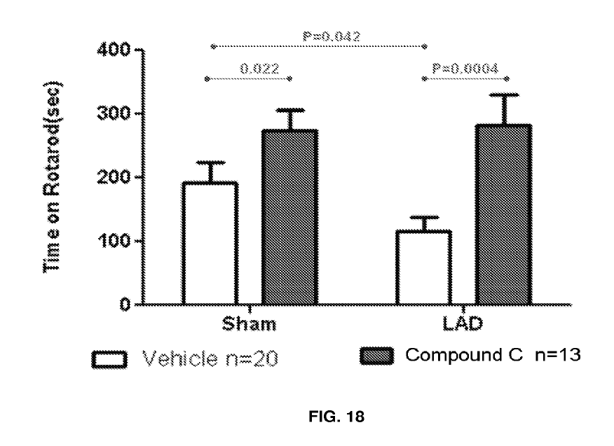

As shown in FIG. 18, vehicle-treated sham rats ran longer than vehicle-treated LAD-HF rats (198±26 seconds vs. 111±32 seconds, p=0.042, mean±S.E.). FIG. 18 also shows that LAD-HF rats treated with Compound C increased their rotarod running time approximately 2.5-fold compared to vehicle treatment (277±32 seconds vs. 111±32 seconds, p=0.0004 from an ANCOVA model controlling for baseline, rat cohort, treatment day and sequence). Thus, administration of Compound C increased the fatigue resistance in this rat model of heart failure.

### Example 14

### In vivo Effect of a Fast Skeletal Troponin Activator in a Rat Heart Failure Model

An in situ assessment of selected animals from Example 12 was run at the end of the study to assess functional characteristics of extensor digitorum longus (EDL) muscles in Sham and LAD animals. Effects with and without Compound C (3 mg/kg IV) were assessed.

Rats were placed under anesthesia and the skin around the experimental leg was removed. The distal end of the extensor digitorum longus (EDL) muscle and its associated tendon were then isolated. The rat was then placed on the platform of an Aurora in situ muscle analysis rig wherein the knee was immobilized and the distal tendon cut and tied to the arm of a force transducer. The muscle was stimulated directly via steel needle electrodes contacting the peroneal nerve. Muscle contractile properties were assessed by applying an electrical current to the nerve and recording the force generated by the muscle via a servomotor. The muscle length was adjusted to produce the maximum isometric force (Lₒ) after sub-maximal stimulation (30 Hz, 1 ms pulses, 350 ms train duration). Once Lₒ had been established, the nerve was stimulated every 2 minutes with a 30 Hz train (1 ms stimuli, 350 ms duration) for the course of the experiment.

Once the preparation was stable, a force frequency relationship was assessed, then Compound C (3 mg/kg IV) or vehicle was injected and a second force frequency was assessed. Stimulation frequency was set at that which produced 50% of maximum force and a 5 minute fatigue stimulation protocol of 1 train per second was run over 5 minutes.

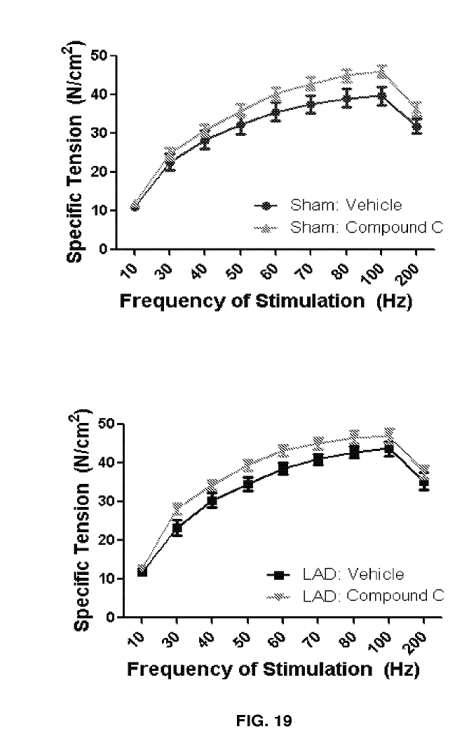

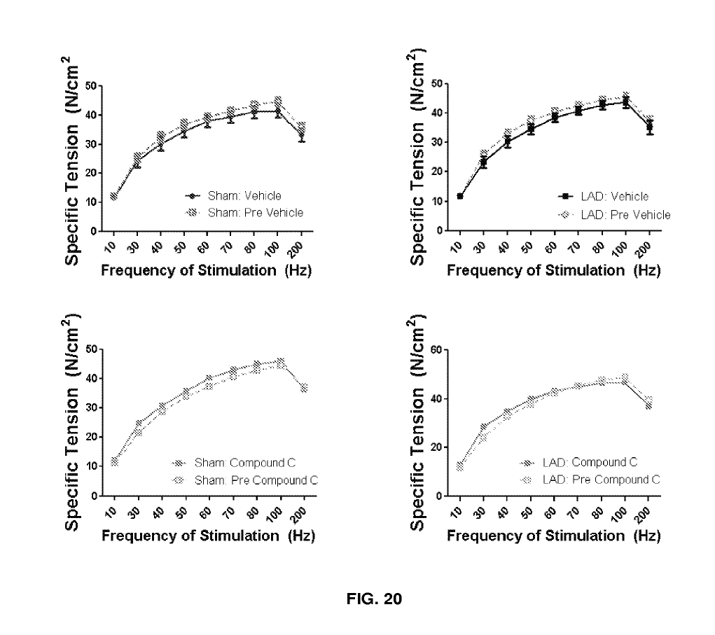

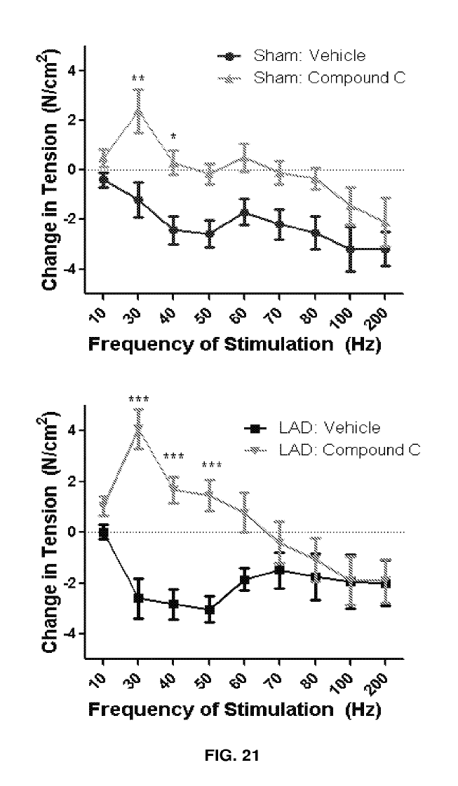

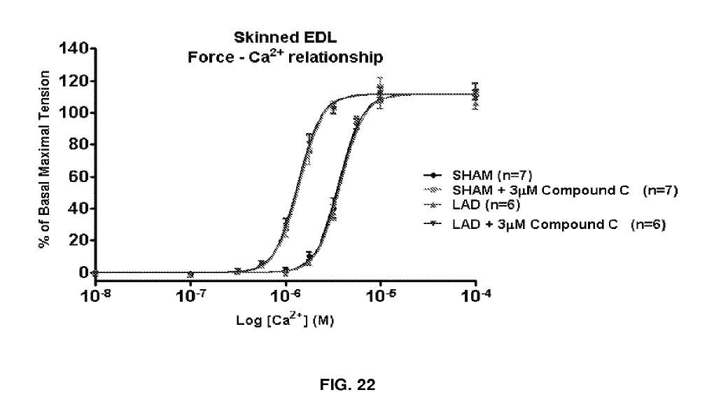

The results of this experiment showed little difference between LAD-HF and sham animals, in general. Compound C increased response to low frequency stimulation in both groups, although the increase was slightly larger in the LAD-HF group (FIG. 19 and FIG. 20). When the baseline tension was subtracted from the second tension measurements in the presence of vehicle, it was apparent that the second force-frequency relationship was substantially lower, likely due to fatigue of the muscle (FIG. 21). However, in the presence of Compound C, the second force frequency curve was increased in both the sham surgery EDL muscle and the LAD-HF EDL muscle (FIG. 21). The response to Compound C was greater in the LAD-HF rat muscle compared to sham.

### EXAMPLE 15

### In vitro Effect of a Fast Skeletal Troponin Activator on Skinned Muscle Fibers in a Rat Heart Failure Model

General Procedure for Skinned Fiber Studies:

Muscle tissue for in vitro skinned fiber studies were prepared using an adapted protocol based on Lynch and Faulkner (Am J Physiol 275:C1548-54 (1998)). Briefly, rat muscle from sham and HF animals were rapidly dissected, rinsed in physiological saline, and then incubated in skinning solution (125 mM K-propionate, 20 mM imidazole, 5 mM EGTA, 2 mM MgCl₂, 2 mM ATP, pH 7.0) supplemented with 0.5% TritonX-100 (Sigma Chemicals, St. Louis, Mo.) for 30 minutes at 4° C. The buffer was then changed to a storage solution (125 mM K-propionate, 20 mM imidazole, 5 mM EGTA, 2 mM MgCl₂, 2 mM ATP, glycerol 50%, pH 7.0) and stored at −20° C. for later use.

For skinned fiber analysis, single muscle fibers were dissected from larger segments of tissue in rigor buffer at 4° C. (20 μM MOPS, 5 μM MgCl2, 120 μM potassium acetate, 1 μM EGTA, pH 7.0). They were then suspended between a 400 A force transducer (Aurora Scientific, Ontario, Canada) and a fixed post and secured with 2-4 μl of a 5% solution of methylcellulose in acetone. Fibers were then incubated at 10° C. in a relaxing buffer (20 μM MOPS, 5.5 μM MgCl₂, 132 μM potassium acetate, 4.4 μM ATP, 22 μM creatine phosphate, 1 mg/ml creatine kinase, 1 mM DTT, 44 ppm antifoam, pH 7.0) and baseline tension adjusted. Tension was generated in each fiber by changing fiber buffer over to relax buffer supplemented with 1 mM EGTA and a 15 mM solution of calcium chloride and calculated using the web resource (www.stanford.edu/˜cpatton/webmaxc/webmaxcS.htm). Compound A was added to these buffers from a DMSO solution (final DMSO concentration=1%).

EDL muscles were harvested from sham and HF animals as described above. As shown in FIG. 22, there was no difference in the force-pCA relationship between SHAM and HF EDL fibers. However, 3 μM Compound C significantly caused a leftward shift in the force-Ca²⁺ relationship in both sham and HF EDL muscle.

### EXAMPLE 16

### In vitro Effect of Fast Skeletal Troponina on Skinned Diaphragm Muscle Fiber in a Rat Heart Failure Model

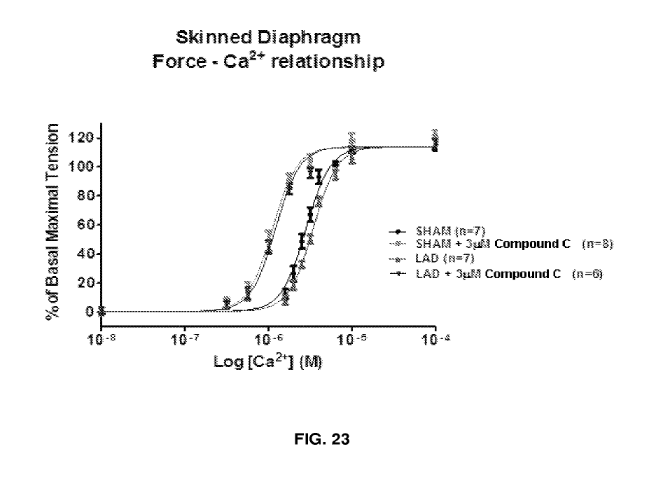

Diaphragm muscles were harvested from sham and LAD-HF rats as described in Example 14. Compared to sham diaphragms, LAD-HF diaphragm fibers had significantly lower Ca²⁺ sensitivity. 3 μM Compound C significantly increased Ca²⁺ sensitivity in both sham and LAD-HF diaphragm fibers (FIG. 23).

### EXAMPLE 17

### In vitro Effect of a Fast Skeletal Troponin Activator on Diaphragm Force-Frequency Relationship in a Rat Heart Failure Model

Diaphragm contractile force was measured by electrical field stimulation in an organ bath system based on a standard operating protocol adapted from the Treat NMD website (http://www.treat-nmd.eu/downloads/file/sops/dmd/MDX/DMD M.1.2.002.pdf). The diaphragm and the last floating rib from sham and HF animals were excised, rinsed in physiological saline, and placed in a temperature controlled water-jacketed chamber (26-27° C.) containing Krebs-Henseleit Buffer (118 mM NaCl, 10 mM glucose, 4.6 mM KCl, 1.2 mM KH₂PO₄, 1.2 mM MgSO₄*7 H₂O, 24.8 mM NaHCO₃, 2.5 mM CaCl₂, 50 mg/L tubocurarine, 50 U/L insulin, pH:7.4) that was continuously aerated with 95% O₂/5% O₂. After 10 minutes of equilibration, vertical strips spanning the floating rib to the central tendon were cut from diaphragms. Braided silk sutures were tied at the central tendon and floating rib and attached to a force transducer between two platinum electrodes. Diaphragm strips were set to a length that produced maximum twitch tension (Lₒ). The force-frequency profile of the muscle was obtained by stimulating the muscle at frequencies between 10-150 Hz (Grass Stimulator, 800 ms train duration, 0.6 ms pulse width). Compound C was suspended in DMSO and directly added into the bath.

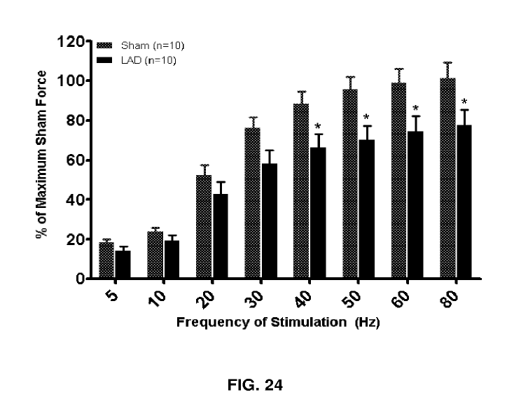

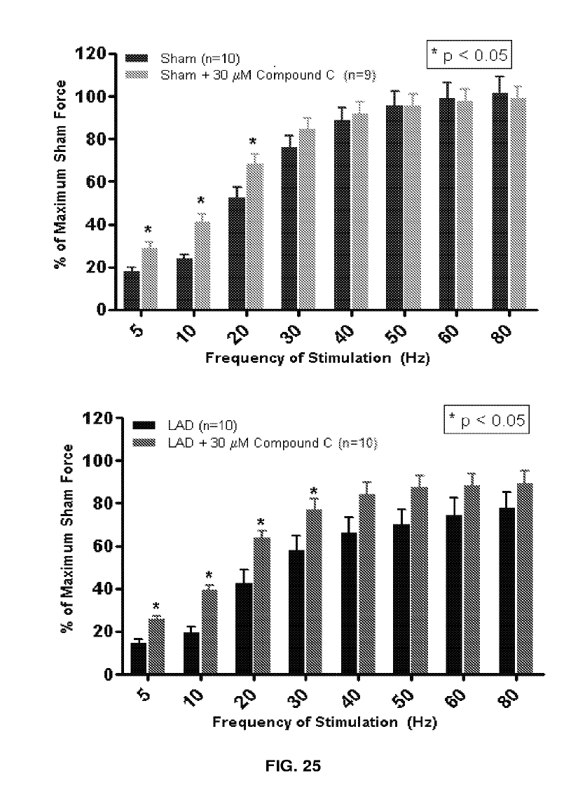

As shown in FIG. 24, LAD-HF diaphragm muscle produced significantly lower force compared to sham diaphragms. 30 μM Compound C significantly increased force in both sham (FIG. 25, top panel) and LAD-HF (FIG. 25, bottom panel) diaphragms at submaximal frequencies of electrical stimulation. Thus, these studies indicate that increasing diaphragm muscle Ca²⁺ sensitivity by administration of a troponin activator such as Compound C improves the tension output in a weakened diaphragm.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1. A method of improving resistance to skeletal muscle fatigue in a subject, the method comprising administering to the subject a therapeutically effective amount of a skeletal muscle troponin activator.

2. The method of claim 1, wherein the subject is suffering from a condition selected from myocardial infarction, anemia and muscle ischemia.

3. The method of claim 1, wherein the subject is suffering from heart failure.

4. A method of improving resistance to fatigue in a skeletal muscle, the method comprising contacting the skeletal muscle with a skeletal muscle troponin activator.

5. The method of claim 1, wherein the skeletal muscle troponin activator increases submaximal tension in the skeletal muscle.

6. The method of claim 1, wherein the skeletal muscle troponin activator reduces the intracellular calcium required by the skeletal muscle to generate force.

7-13. (canceled)

14. A method for treating exercise intolerance in a patient suffering from heart failure comprising administering to the patient a therapeutically effective amount of a skeletal muscle troponin activator.

15. A method for improving physical endurance performance of a patient suffering from heart failure, comprising administering to the subject a therapeutically effective amount of a skeletal muscle troponin activator.

16. A method for increasing the function, activity, efficiency, sensitivity to calcium, or time to fatigue of skeletal muscle of a patient suffering from heart failure, comprising administering to the patient a therapeutically effective amount of a skeletal muscle troponin activator.

17. A method for improving skeletal muscle function of a patient suffering from heart failure, comprising administering to the patient a therapeutically effective amount of a skeletal muscle troponin activator.

18. The method of claim 14, further comprising administering to the subject a second therapy.

19. The method of claim 18, wherein the second therapy is selected from an antiplatelet drug, a diuretic, a calcium channel blocker, a beta blocker, an ACE inhibitor, a statin, an angiotensin II receptor antagonist, and an aldosterone antagonist.

20. The method of claim 19, wherein the second therapy is selected from digoxin, aspirin, ticlopidine, clopidogrel, metoprolol, carvedilol, eplerenone, and spironolactone.

21. The method of claim 18, wherein the second therapy is selected from angioplasty, stenting, and surgery.

22. The method of claim 18 wherein the skeletal muscle troponin activator and the second therapy are administered simultaneously to the subject.

23. The method of claim 18 wherein the skeletal muscle troponin activator and the second therapy are administered sequentially to the subject.

24. The method of claim 1, wherein the skeletal muscle troponin activator is a fast skeletal muscle troponin activator.

25-26. (canceled)

27. The method of claim 1, wherein the skeletal muscle troponin activator is selected from compounds of Formula I: 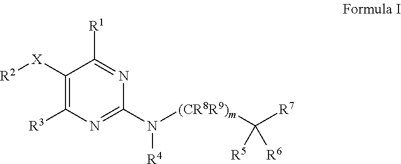
or a pharmaceutically acceptable salt thereof, wherein:
R¹ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;
R² is selected from C₃₋₈ cycloalkyl, C₃₋₈ cycloalkenyl, 3-8 membered heterocycloalkyl, 3-8 membered heterocycloalkenyl, C₆₋₁₀ aryl, 5-10 membered heteroaryl and NR^{b}R^{c}, wherein each of the C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl groups is optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)NR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl groups is optionally substituted with 1, 2, 3, 4 or 5 R^{f} substituents;
R³ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;
R⁴ is selected from hydrogen, C₁₋₆ alkyl, C₁₋₆ haloalkyl, C(O)R^{a}, C(O)OR^{a}, C(O)NR^{b}R^{c} and SO₂R^{a};
R⁵ and R⁶ are each independently selected from hydrogen, halogen, C₁₋₆ alkyl and C₁₋₆ haloalkyl;
or alternatively, R⁵ and R⁶ together with the carbon atom to which they are bound form a group selected from C₃₋₈ cycloalkyl, C₃₋₈ cycloalkenyl, 3-8 membered heterocycloalkyl and 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl;
R⁷ is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{c})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;
R⁸ and R⁹, at each occurrence, are each independently selected from hydrogen, halogen and C₁₋₆ alkyl;
X is selected from a bond, —(CH₂)ₚ—, —(CH₂)ₚC(O)(CH₂)_{q}—, —(CH₂)ₚO(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚOC(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—;
or alternatively, X, R² and R³, together with the carbon atoms to which they are bound, form a 5-6 membered ring optionally containing one or more heteroatoms selected from oxygen nitrogen and sulfur, and optionally containing one or more double bonds, and optionally substituted with 1, 2, 3, 4 or 5 R^{f} substituents;
R^{a}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;
R^{b} and R^{c}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, C(O)OR^{g}, C(O)NRⁱR^{j} and SO₂R^{g}, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;
R^{d}, at each occurrence, is independently selected from hydrogen and C₁₋₆ alkyl;
R^{e}, at each occurrence, is independently selected from hydrogen, CN, OH, C₁₋₆ alkoxy, C₁₋₆ alkyl and C₁₋₆ haloalkyl;
R^{f}, at each occurrence, is independently selected from halogen, CN, OR^{h}, OC(O)R^{h}, OC(O)OR^{h}, OC(O)NRⁱR^{j}, NRⁱR^{j}, NR^{d}C(O)R^{h}, NR^{d}C(O)R^{h}, NR^{d}C(O)NRⁱR^{j}, NR^{d}C(O)C(O)NRⁱR^{j}, NR^{d}C(S)R^{h}, NR^{d}C(S)OR^{h}, NR^{d}C(S)NRⁱR^{j}, NR^{d}C(NR^{e})NRⁱR^{j}, NR^{d}S(O)R^{h}, NR^{d}SO₂R^{h}, NR^{d}SO₂NRⁱR^{j}, C(O)R^{h}, C(O)OR^{h}, C(O)NRⁱR^{j}, C(S)R^{h}, C(S)OR^{h}, C(S)NRⁱR^{j}, C(NR^{e})NRⁱR^{j}, SR^{h}, S(O)R^{h}, SO₂R^{h}, SO₂NRⁱR^{j}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;
or two R^{f} substituents bound to a single carbon atom, together with the carbon atom to which they are both bound, form a group selected from carbonyl, C₃₋₈ cycloalkyl and 3-8 membered heterocycloalkyl;
R^{g}, at each occurrence, is independently selected from C₁₋₆ alkyl, C₁₋₆ haloalkyl, phenyl, naphthyl, and C₇₋₁₁ aralkyl, each optionally substituted with 1, 2, 3, 4 or 5 substituents selected from halogen, CN, OH, C₁₋₆ alkoxy, C₁₋₆ alkyl and C₁₋₆ haloalkyl;
R^{h}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;
Rⁱ and R^{j}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, and C(O)OR^{g}, wherein each of the C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 substituents selected from halogen, CN, OH, C₁₋₆ alkoxy, C₁₋₆ alkyl and C₁₋₆ haloalkyl;
R^{k}, at each occurrence, is independently selected from halogen, CN, OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, NHC(O)OC₇₋₁₁ aralkyl, OC(O)C₁₋₆ alkyl, OC(O)C₇₋₁₁ aralkyl, OC(O)OC₁₋₆ alkyl, OC(O)OC₇₋₁₁ aralkyl, C(O)C₁₋₆ alkyl, C(O)C₇₋₁₁ aralkyl, C(O)OC₁₋₆ alkyl, C(O)OC₇₋₁₁ aralkyl, C₁₋₆ alkyl, C₁₋₆ haloalkyl, C₂₋₆ alkenyl, and C₂₋₆ alkynyl, wherein each C₁₋₆ alkyl, C₂₋₆ alkenyl, C₂₋₆ alkynyl, and C₇₋₁₁ aralkyl substituent is optionally substituted with 1, 2 or 3 substituents selected from OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, and NHC(O)OC₇₋₁₁ aralkyl;
or two R^{k} substituents bound to a single carbon atom, together with the carbon atom to which they are both bound, form a carbonyl group;
m is 0, 1 or 2;
n, at each occurrence, independently is 0, 1 or 2;
p is 0, 1 or 2; and
q is 0, 1 or 2.

28-34. (canceled)

35. The method of claim 27, wherein the compound of Formula I, or the pharmaceutically acceptable salt thereof, is a compound of Formula XII(o): 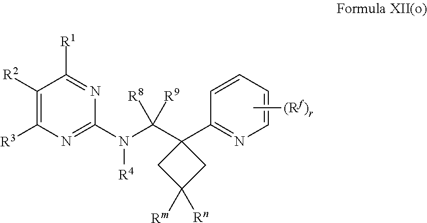
or a pharmaceutically acceptable salt thereof, wherein:
R¹ is hydrogen;
R² is selected from furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with
(CH₂)ₙC(O)NH₂;
R³ is hydrogen;
R⁴ is hydrogen;
R⁸ and R⁹ are each hydrogen;
one of R^{m} and Rⁿ is hydrogen and the other is fluorine;
R^{f}, at each occurrence, is independently halogen;
n is 0, 1 or 2; and
r is 0, 1, 2, 3 or 4.

36. The method of claim 35, wherein one of R^{m} and Rⁿ is hydrogen and the other is fluorine; and
the fluorine and the pyridyl ring are in a trans configuration with respect to one another on the cyclobutyl ring.

37. The method of claim 27, wherein the compound of Formula I, or the pharmaceutically acceptable salt thereof, is
1-(2-(((trans)-3-fluoro-1-(3-fluoropyridin-2-yl)cyclobutyl)methylamino)pyrimidin-5-yl)-1H-pyrrole-3-carboxamide, or a pharmaceutically acceptable salt thereof, wherein
1-(2-(((trans)-3-fluoro-1-(3-fluoropyridin-2-yl)cyclobutyl)methylamino)pyrimidin-5-yl)-1H-pyrrole-3-carboxamide is defined by the following structure: 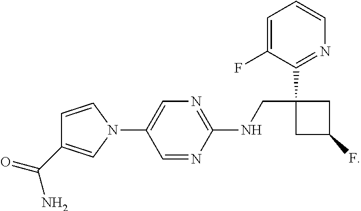 [SMILES: NC(=O)c1ccn(-c2cnc(NC[C@]3(c4ncccc4F)C[C@H](F)C3)nc2)c1]
